SEQUENCE LISTING (154 sequences)

SEQ ID NO: 1 - DNA, 2579 bp, homo sapiens
gaaatatgaagagacgctgcagctgcggtggcggtggcggccactgcagctcagagcggcgcacgcggcggccggggcgggacgcggggccgggcgcggagaagtcggggcgggcggcagagaggccgggacgcggaccgggccggggcgcccacagccgcccgacggcgcccagagagcgcgcgccccgcagccccgcgcctagcccgccgggcatggggcgcgcggcagccgcctgaagccccggcctggcccggccgcacccggccggaggcggagggcagagcgcgcgcccagttgcccgggcaccaaatcggagcgcggcgtgcgggaggccccagagcaggactggaaatgtcctggccgcgccgcctcctgctcagatacctgttcccggccctcctgcttcacgggctgggagagggttctgccctccttcatccagacagcaggtctcatcctaggtccttagagaaaagtgcctggagggcttttaaggagtcacagtgccatcacatgctcaaacatctccacaatggtgcaaggatcacagtgcagatgccacctacaatcgagggccactgggtctccacaggctgtgaagtaaggtcaggcccagagttcatcacaaggtcctacagattctaccacaataacaccttcaaggcctaccaattttattatggcagcaaccggtgcacaaatcccacttatactctcatcatccggggcaagatccgcctccgccaggcctcctggatcatccgagggggcacggaagccgactaccagctgcacaacgtccaggtgatctgccacacagaggcggtggccgagaagctcggccagcaggtgaaccgcacatgcccgggcttcctcgcagacgggggtccctgggtgcaggacgtggcctatgacctctggcgagaggagaacggctgtgagtgcaccaaggccgtgaactttgccatgcatgaacttcagctcatccgggtggagaagcagtaccttcaccacaacctcgaccacctggtcgaggagctcttccttggtgacattcacactgatgccacccagaggatgttctaccggccctccagttaccagccccctctgcagaatgccaagaaccacgaccatgcctgcatcgcctgtcggatcatctatcggtcagacgagcaccaccctcccatcctgcccccaaaggcagacctgaccatcggcctgcacggggagtgggtgagccagcgctgtgaggtgcgccccgaagtcctcttcctcacccgccacttcatcttccatgacaacaacaacacctgggagggccactactaccactactcagacccggtgtgcaagcaccccaccttctccatctacgcccggggccgctacagccgcggcgtcctctcgtccagggtcatgggaggcaccgagttcgtgttcaaagtgaatcacatgaaggtcacccccatggatgcggccacagcctcactgctcaacgtcttcaacgggaatgagtgcggggccgagggctcctggcaggtgggcatccagcaggatgtgacccacaccaatggctgcgtggccctgggcatcaaactacctcacacggagtacgagatcttcaaaatggaacaggatgcccgggggcgctatctgctgttcaacggtcagaggcccagcgacgggtccagcccagacaggccagagaagagagccacgtcctaccagatgcccttggtccagtgtgcctcctcttcgccgagggcagaggacctcgcagaagacagtggaagcagcctgtatggccgggcccctgggaggcacacctggtccctgctgctggctgcacttgcctgccttgtccctctgctgcattggaacatccgcagatagaagttttagaaagttctatttttccaaaccaggattccttactattgacagatttgctttaccaaaagaaaagacatttattcttttgatgcacttgaatgccagagaactgtccttctttttctcctctccctccctcccagcccctgagtcatgaacagcaaggagtgtttgaagtttctgctttgaactccgtccagcctgatccctggcctgagcaacttcacaacagtaattgcactttaagacagcctagagttctggacgagcgtgtttggtagcagggatgaaagctagggcctcttatttttttctcttaattattattatatttctgagttaaacttagaagaaacaactatcaagctacaacttttcctgccattttcctgtggttgcagcctgtcttcctttgaaattgttttactctctgagttttatatgctggaatccaatgcagagttggtttgggactgtgatcaagacaccttttattaataaagaagagacacaggtgtagatatgtatatacaaaaagatgtacggtctggccaaaccaccttcccagcctttatgcaaaaaaaggggagaatcaaagctttcatttcagaaatgttgcgtggaaaagtatctgtaattaaagtttcgaagtaatttaacctaaaaaaaaaaaaaaaaaa

SEQ ID NO: 2 - protein, 514 aa, homo sapiens
MSWPRRLLLRYLFPALLLHGLGEGSALLHPDSRSHPRSLEKSAWRAFKESQCHHMLKHLHNGARITVQMPPTIEGHWVSTGCEVRSGPEFITRSYRFYHNNTFKAYQFYYGSNRCTNPTYTLIIRGKIRLRQASWIIRGGTEADYQLHNVQVICHTEAVAEKLGQQVNRTCPGFLADGGPWVQDVAYDLWREENGCECTKAVNFAMHELQLIRVEKQYLHHNLDHLVEELFLGDIHTDATQRMFYRPSSYQPPLQNAKNHDHACIACRIIYRSDEHHPPILPPKADLTIGLHGEWVSQRCEVRPEVLFLTRHFIFHDNNNTWEGHYYHYSDPVCKHPTFSIYARGRYSRGVLSSRVMGGTEFVFKVNHMKVTPMDAATASLLNVFNGNECGAEGSWQVGIQQDVTHTNGCVALGIKLPHTEYEIFKMEQDARGRYLLFNGQRPSDGSSPDRPEKRATSYQMPLVQCASSSPRAEDLAEDSGSSLYGRAPGRHTWSLLLAALACLVPLLHWNIRR

SEQ ID NO: 3 - DNA, 3112 bp, homo sapiens
acaactatcaacagccgggaaggctgagcgcgtgtgagcgccgaggggggcgcaggaccctcgcaacttcttcgcaggactccagcctggccgccggcgcccgcagccgtccgagagccctgcgcccgcgcctccccttgcgcaccgtggcagcgcccggcgggcggtcctgccagccccgacgggatgcccgcagccatgctcccctacgcttgcgtcctggtgcttttgggagcccacactgcaccggcggctggggaggccgggggcagctgcctgcgctgggaaccccactgccagcagcccttgccagatagagtgcccagcactgcgatcctgcctccacgccttaatggaccttggatctccacaggctgcgaggtgcgcccaggaccggagttcctgacccgcgcctacaccttctaccccagccggctctttcgagcccaccagttctactacgaggaccccttctgcggggaacctgcccactcgctgctcgtcaagggcaaagtccgcctgcgccgggcctcctgggtcacccggggagccaccgaggccgactaccacctgcacaaggtgggcatcgtcttccacagccgccgggccctggtcgacgtcaccgggcgcctcaaccagacccgcgccggccgggactgcgcgcggcggctgcctccggcccgggcctggctgcctggggcgctgtacgagctgcggagcgcccgggctcagggggactgcctggaggcgctgggcctcaccatgcacgagctcagcctggtccgcgtgcagcgccgcctgcagccgcagccccgggcgtcgccccggctggtggaggagctgtacctgggggacatccacaccgacccggcggagaggcggcactaccggcccacgggctaccagcgcccgctgcagagcgcactgcaccacgtgcagccgtgcccagcctgtggcctcattgcccgctccgatgtgcaccacccgcccgtgctgccgccccctctggccctgcccctgcacctgggcggctggtgggtcagctcggggtgcgaggtgcgcccagcagtcctgttcctcacccggctcttcactttccacgggcacagccgctcctgggaagggtattaccaccacttctcagacccagcctgccggcagcccaccttcaccgtgtatgccgccggccgctacaccaggggcacgccatccaccagggtccgcggcggcaccgagctggtgtttgaggtcacacgggcccatgtgacccccatggaccaggtcaccacggccatgctcaacttctctgagccaagcagctgtgggggtgcgggggcctggtccatgggcactgagcgggatgtcacagccaccaacggctgcctaccgctgggcatccggctcccgcatgtggagtacgagcttttcaagatggaacaagaccccctcgggcaaagcctgctcttcatcggacaaaggcccaccgatggctcaagtcccgataccccagagaaacgtcccacctcctaccaagcacccctggtgctctgtcatggggaggcccccgacttctccaggccaccgcagcacaggccatcgctgcagaagcaccccagcacagggggtcttcacatagcccccttcccacttctgcccctagttctagggctggccttcctccactggctatgacattggacttgacatcaggatggcggctctggacacccattcaacccttcagactccctcctggcagctgtagggaaggaaccattctcctctgctctgtcatggatggatgcacagccccactgcttccaaactctgcctgtgtcccatgtggctcaggacatgagcttaacccctgcaaagcctataccacatcccacagcccgggtccccagtcaagcacttggatgcggcagtgatgttcatcgctacgtgagtttctaaagatcactcccaatttttctactttcctcatccttggcagctcgccaacaggttcagtcagggggccacacggaacacccccatcccatgttccccccagttcttcccatcctgacccttgggattccaagatgggagcaagaggagatcctgaggctctgcctagggacgaggcctacagttctgccatgtctgtaggttgttgtttaaagattattaattcgaatttagcaatacgatctctaagtggtgccatgaattaaagatgccacttcgggctttcagtgcttctcagcttttgggcaaagggcttgtgtcttcaggggcagctcagctttcctgagtcctgactgctggcactcgtctgcatttgcctgtgcttctgcgagtcggacctcaagctgccaacactgcatgtggataaatccagttttcccgggccagcatgcaaaatgaagaggactccatctaagctgagaagcatggcctccccagagcagcctgcggcctccaagccttcctggcccaggcaaatgccagtgtgcaccaggctggctgctgggggcaggtctttggaggggagcagcatttccagccttctgaacatagttaatagtaatgacagccgtaacactaacgcgctctgcaattcgccctgcccagccatcctcggttgccaagattgcctgtgcctgcctgacaaaggaagagaatctccgaatgtgtatctttgggcccaccctagggagaggtcggggtcaccaggctacatggcgacatctaggcagctccgccttgcccagcctccttgccataatcctaatatattggtgtcctctgctcagaggggactgtcatcatggtgggaacaggctgtgcctccccagggactctgcccatgttcccagggcctcatctgtacactgtgaaattaactggcatcctggtgggcccaagggttttcaggactgggggccaatgactcaccccctccttcctcctcctgatccctatctctagctcttatcacagattttgaacaattgtctgtgaggttaatgatggtttcagagggaagcccttttcctccctgagactgtgtggggttcagtcagcctgctgaaattgcttccacttattacccatccttcctctt

SEQ ID NO: 4 - protein, 501 aa, homo sapiens
MPAAMLPYACVLVLLGAHTAPAAGEAGGSCLRWEPHCQQPLPDRVPSTAILPPRLNGPWISTGCEVRPGPEFLTRAYTFYPSRLFRAHQFYYEDPFCGEPAHSLLVKGKVRLRRASWVTRGATEADYHLHKVGIVFHSRRALVDVTGRLNQTRAGRDCARRLPPARAWLPGALYELRSARAQGDCLEALGLTMHELSLVRVQRRLQPQPRASPRLVEELYLGDIHTDPAERRHYRPTGYQRPLQSALHHVQPCPACGLIARSDVHHPPVLPPPLALPLHLGGWWVSSGCEVRPAVLFLTRLFTFHGHSRSWEGYYHHFSDPACRQPTFTVYAAGRYTRGTPSTRVRGGTELVFEVTRAHVTPMDQVTTAMLNFSEPSSCGGAGAWSMGTERDVTATNGCLPLGIRLPHVEYELFKMEQDPLGQSLLFIGQRPTDGSSPDTPEKRPTSYQAPLVLCHGEAPDFSRPPQHRPSLQKHPSTGGLHIAPFPLLPLVLGLAFLHWL

SEQ ID NO: 5 - DNA, 20 bp, Artificial Sequence
  synthetic oligonucleotide
ccagagcaggactggaaatg

SEQ ID NO: 6 - DNA, 19 bp, Artificial Sequence
  synthetic oligonucleotide
cgccaaggggacagtgtag

SEQ ID NO: 7 - protein, 20 aa, Artificial Sequence
  synthetic peptide
CQRPSDGSSPDRPEKRATSY

SEQ ID NO: 8 - DNA, 30 bp, Artificial Sequence
  synthetic oligonucleotide
aaaactcgagccagagcaggactggaaatg

SEQ ID NO: 9 - DNA, 32 bp, Artificial Sequence
  synthetic oligonucleotide
aaaagctagcctatctgcggatgttccaatgc

SEQ ID NO: 10 - DNA, 59 bp, Artificial Sequence
  synthetic oligonucleotide
aaaagctagctcaggcgtagtcgggcacgtcgtaggggtatctgcggatgttccaatgc

SEQ ID NO: 11 - DNA, 56 bp, Artificial Sequence
  synthetic oligonucleotide
aaaagctagctcacttatcgtcgtcatccttgtaatctctgcggatgttccaatgc

SEQ ID NO: 12 - DNA, 58 bp, Artificial Sequence
  synthetic oligonucleotide
aaaactcgagccagagcaggactggaaatgtcctggccgcgccgcctcctgcgcagat

SEQ ID NO: 13 - DNA, 58 bp, Artificial Sequence
  synthetic oligonucleotide
aaaactcgagccagagcaggactggaaatgtcctggccgcgccgcctcctggtcagat

SEQ ID NO: 14 - DNA, 61 bp, Artificial Sequence
  synthetic oligonucleotide
aaaagctagctcaggcgtagtcgggcacgtcgtaggggtagccatacaggctgcttccact

SEQ ID NO: 15 - DNA, 58 bp, Artificial Sequence
  synthetic oligonucleotide
aaaagctagctcacttatcgtcgtcatccttgtaatcgccatacaggctgcttccact

SEQ ID NO: 16 - DNA, 54 bp, Artificial Sequence
  synthetic oligonucleotide
aaaacctaggcttatcgtcgtcatccttgtaatcatgagacctgctgtctggat

SEQ ID NO: 17 - DNA, 31 bp, Artificial Sequence
  synthetic oligonucleotide
aaaagaattcccttcatccagacagcaggtc

SEQ ID NO: 18 - DNA, 34 bp, Artificial Sequence
  synthetic oligonucleotide
aaaactcgagtcagccatacaggctgcttccact

SEQ ID NO: 19 - DNA, 39 bp, Artificial Sequence
  synthetic oligonucleotide
ccatcgatggccaccatgggaatcccctgtttctactgc

SEQ ID NO: 20 - DNA, 37 bp, Artificial Sequence
  synthetic oligonucleotide
ccaccgtcgacgcttagaatttccaaatgtagcaaag

SEQ ID NO: 21 - DNA, 20 bp, Artificial Sequence
  synthetic oligonucleotide
ggggacagagacggactaca

SEQ ID NO: 22 - DNA, 19 bp, Artificial Sequence
  synthetic oligonucleotide
caaggcattcaagtgcatc

SEQ ID NO: 23 - DNA, 20 bp, Artificial Sequence
  synthetic oligonucleotide
ggggacagagacggactaca

SEQ ID NO: 24 - DNA, 35 bp, Artificial Sequence
  synthetic oligonucleotide
ctgccctgcctgccatacagatgaccttgactgtc

SEQ ID NO: 25 - DNA, 35 bp, Artificial Sequence
  synthetic oligonucleotide
aaaactcgagcggcgatggccccactcggatactt

SEQ ID NO: 26 - DNA, 33 bp, Artificial Sequence
  synthetic oligonucleotide
aaaagctagcctacttgcaggtgtgcacgtcgt

SEQ ID NO: 27 - DNA, 59 bp, Artificial Sequence
  synthetic oligonucleotide
aaaagctagctaggcgtagtcgggcacgtcgtaggggtacttgcaggtgtgcacgtcgt

SEQ ID NO: 28 - DNA, 32 bp, Artificial Sequence
  synthetic oligonucleotide
atatctcgagcctcgctatggttcgtctgcct

SEQ ID NO: 29 - DNA, 58 bp, Artificial Sequence
  synthetic oligonucleotide
atatgctagctaggcgtagtcgggcacgtcgtaggggtatctcagccgatcctgggga

SEQ ID NO: 30 - DNA, 28 bp, Artificial Sequence
  synthetic oligonucleotide
aaaagaattccggacaacatggaggcag

SEQ ID NO: 31 - DNA, 60 bp, Artificial Sequence
  synthetic oligonucleotide
aaaagctagctacttatcgtcgtcatccttgtaatcgctgtgggccgggctgtcgtctga

SEQ ID NO: 32 - DNA, 25 bp, Artificial Sequence
  synthetic oligonucleotide
tggtagttcagctccagaatgtcct

SEQ ID NO: 33 - DNA, 18 bp, Artificial Sequence
  synthetic oligonucleotide
cgaaggctaaagttgcag

SEQ ID NO: 34 - DNA, 18 bp, Artificial Sequence
  synthetic oligonucleotide
aatggatttcagagacca

SEQ ID NO: 35 - DNA, 18 bp, Artificial Sequence
  synthetic oligonucleotide
tcttattccagaggaacc

SEQ ID NO: 36 - DNA, 18 bp, Artificial Sequence
  synthetic oligonucleotide
acaactggaagcactgga

SEQ ID NO: 37 - DNA, 20 bp, Artificial Sequence
  synthetic oligonucleotide
ctggagctgaactaccatgg

SEQ ID NO: 38 - DNA, 21 bp, Artificial Sequence
  synthetic oligonucleotide
tgaccctcgatgtttggaggc

SEQ ID NO: 39 - DNA, 24 bp, Artificial Sequence
  synthetic oligonucleotide
gcaaatgaatacactaactagcca

SEQ ID NO: 40 - DNA, 21 bp, Artificial Sequence
  synthetic oligonucleotide
caccaggcctaggctaaaatg

SEQ ID NO: 41 - DNA, 23 bp, Artificial Sequence
  synthetic oligonucleotide
ccagtgagaccttaaaagcttca

SEQ ID NO: 42 - DNA, 23 bp, Artificial Sequence
  synthetic oligonucleotide
ttgctatactttaggagccagaa

SEQ ID NO: 43 - DNA, 22 bp, Artificial Sequence
  synthetic oligonucleotide
ggcttgggtacaataatgagct

SEQ ID NO: 44 - DNA, 20 bp, Artificial Sequence
  synthetic oligonucleotide
cagagcttcctgcacttcac

SEQ ID NO: 45 - DNA, 20 bp, Artificial Sequence
  synthetic oligonucleotide
cgacgcgccctttcaagtct

SEQ ID NO: 46 - DNA, 19 bp, Artificial Sequence
  synthetic oligonucleotide
cgccaaggggacagtgtag

SEQ ID NO: 47 - DNA, 19 bp, Artificial Sequence
  synthetic oligonucleotide
cactgtctgctgcagaggt

SEQ ID NO: 48 - DNA, 20 bp, Artificial Sequence
  synthetic oligonucleotide
ggtcttccatagtggtgtgc

SEQ ID NO: 49 - DNA, 23 bp, Artificial Sequence
  synthetic oligonucleotide
gaatctctttcccataccttcag

SEQ ID NO: 50 - DNA, 22 bp, Artificial Sequence
  synthetic oligonucleotide
tggcaagcctttaagaaggatc

SEQ ID NO: 51 - DNA, 19 bp, Artificial Sequence
  synthetic oligonucleotide
cggaagcatgtgtgcactg

SEQ ID NO: 52 - DNA, 20 bp, Artificial Sequence
  synthetic oligonucleotide
aagaggtcctttccctcagc

SEQ ID NO: 53 - DNA, 21 bp, Artificial Sequence
  synthetic oligonucleotide
gtctagttagagtgtggccag

SEQ ID NO: 54 - DNA, 22 bp, Artificial Sequence
  synthetic oligonucleotide
ttctctggcattcaagtgcatc

SEQ ID NO: 55 - DNA, 21 bp, Artificial Sequence
  synthetic oligonucleotide
caaagcaggagtgttgaagca

SEQ ID NO: 56 - DNA, 22 bp, Artificial Sequence
  synthetic oligonucleotide
tgtgctcactcttctcaaagtg

SEQ ID NO: 57 - DNA, 22 bp, Artificial Sequence
  synthetic oligonucleotide
gagggaaaaccaactgtaacgt

SEQ ID NO: 58 - DNA, 23 bp, Artificial Sequence
  synthetic oligonucleotide
ctacaactttcccttcttggttc

SEQ ID NO: 59 - DNA, 22 bp, Artificial Sequence
  synthetic oligonucleotide
cagacttcattcgtgatctcga

SEQ ID NO: 60 - DNA, 23 bp, Artificial Sequence
  synthetic oligonucleotide
tacaagcagtgccatttggacat

SEQ ID NO: 61 - DNA, 19 bp, Artificial Sequence
  synthetic oligonucleotide
agcctggcctcgtcctaga

SEQ ID NO: 62 - DNA, 20 bp, Artificial Sequence
  synthetic oligonucleotide
aaagcagccgggaaagggaa

SEQ ID NO: 63 - DNA, 22 bp, Artificial Sequence
  synthetic oligonucleotide
gaaaggcagtgttagtagccat

SEQ ID NO: 64 - DNA, 20 bp, Artificial Sequence
  synthetic oligonucleotide
tatttccctccctccagatg

SEQ ID NO: 65 - DNA, 22 bp, Artificial Sequence
  synthetic oligonucleotide
gtttaaggcaaatcccagactt

SEQ ID NO: 66 - DNA, 22 bp, Artificial Sequence
  synthetic oligonucleotide
actgctaaacaacacccacagt

SEQ ID NO: 67 - DNA, 20 bp, Artificial Sequence
  synthetic oligonucleotide
gtcccgtgaggtgaaactta

SEQ ID NO: 68 - DNA, 20 bp, Artificial Sequence
  synthetic oligonucleotide
ggaactgagagctcaacagc

SEQ ID NO: 69 - DNA, 23 bp, Artificial Sequence
  synthetic oligonucleotide
cagtcattcccaatatcatgcag

SEQ ID NO: 70 - DNA, 23 bp, Artificial Sequence
  synthetic oligonucleotide
cacaataccacttatctgctagg

SEQ ID NO: 71 - DNA, 22 bp, Artificial Sequence
  synthetic oligonucleotide
atctgactgctgacatgtactg

SEQ ID NO: 72 - DNA, 23 bp, Artificial Sequence
  synthetic oligonucleotide
tggcctaataacactcacatgtc

SEQ ID NO: 73 - DNA, 23 bp, Artificial Sequence
  synthetic oligonucleotide
cactaatctactttccgtcccta

SEQ ID NO: 74 - DNA, 20 bp, Artificial Sequence
  synthetic oligonucleotide
cgtgggccatactaccaatg

SEQ ID NO: 75 - DNA, 19 bp, Artificial Sequence
  synthetic oligonucleotide
tgtcgcgctgcaccagctt

SEQ ID NO: 76 - DNA, 19 bp, Artificial Sequence
  synthetic oligonucleotide
ggagtgtgacccagaggac

SEQ ID NO: 77 - DNA, 22 bp, Artificial Sequence
  synthetic oligonucleotide
gacctagaaggtcattacaagc

SEQ ID NO: 78 - DNA, 26 bp, Artificial Sequence
  synthetic oligonucleotide
gccttatagaaagcatatgagtaagt

SEQ ID NO: 79 - DNA, 23 bp, Artificial Sequence
  synthetic oligonucleotide
ctgatttgttgccagtagtcaac

SEQ ID NO: 80 - DNA, 20 bp, Artificial Sequence
  synthetic oligonucleotide
gcgaaccagtgtggacctta

SEQ ID NO: 81 - DNA, 20 bp, Artificial Sequence
  synthetic oligonucleotide
tggaaccaactgggtcagtg

SEQ ID NO: 82 - DNA, 24 bp, Artificial Sequence
  synthetic oligonucleotide
ttacagttaggagtaagtcttggt

SEQ ID NO: 83 - DNA, 22 bp, Artificial Sequence
  synthetic oligonucleotide
tatgttgtgcatgagcccattg

SEQ ID NO: 84 - DNA, 22 bp, Artificial Sequence
  synthetic oligonucleotide
cttgccttacagtaaggaagct

SEQ ID NO: 85 - DNA, 20 bp, Artificial Sequence
  synthetic oligonucleotide
ctgcacatgtgtcccagaac

SEQ ID NO: 86 - DNA, 22 bp, Artificial Sequence
  synthetic oligonucleotide
tggtgtgtctacagcttatacc

SEQ ID NO: 87 - DNA, 22 bp, Artificial Sequence
  synthetic oligonucleotide
aagtacatttcacaggggacct

SEQ ID NO: 88 - DNA, 22 bp, Artificial Sequence
  synthetic oligonucleotide
aagctgtagtgggcttactcta

SEQ ID NO: 89 - DNA, 22 bp, Artificial Sequence
  synthetic oligonucleotide
tcccgtggaagttactatagca

SEQ ID NO: 90 - DNA, 23 bp, Artificial Sequence
  synthetic oligonucleotide
ggtgaaattcaatgcaagtggtc

SEQ ID NO: 91 - DNA, 20 bp, Artificial Sequence
  synthetic oligonucleotide
ccaccatggtcatgaggcat

SEQ ID NO: 92 - DNA, 24 bp, Artificial Sequence
  synthetic oligonucleotide
ggatatccctaatctattcccaag

SEQ ID NO: 93 - DNA, 20 bp, Artificial Sequence
  synthetic oligonucleotide
gctccaccacccatcttatg

SEQ ID NO: 94 - DNA, 23 bp, Artificial Sequence
  synthetic oligonucleotide
ctgccttacgcagataacatttg

SEQ ID NO: 95 - DNA, 20 bp, Artificial Sequence
  synthetic oligonucleotide
gaaggtgggacctgactgga

SEQ ID NO: 96 - DNA, 22 bp, Artificial Sequence
  synthetic oligonucleotide
cgttgactattgacccaaacct

SEQ ID NO: 97 - DNA, 24 bp, Artificial Sequence
  synthetic oligonucleotide
gttggaatgatgaaggggaaattc

SEQ ID NO: 98 - DNA, 21 bp, Artificial Sequence
  synthetic oligonucleotide
gttccacaacgattcccactg

SEQ ID NO: 99 - DNA, 22 bp, Artificial Sequence
  synthetic oligonucleotide
cagttaaggaactgcttgagct

SEQ ID NO: 100 - DNA, 22 bp, Artificial Sequence
  synthetic oligonucleotide
tgccttcattgtgagcagaagt

SEQ ID NO: 101 - DNA, 20 bp, Artificial Sequence
  synthetic oligonucleotide
ctcaggatgcagagaagagc

SEQ ID NO: 102 - DNA, 20 bp, Artificial Sequence
  synthetic oligonucleotide
ttcccaccacctcaggccat

SEQ ID NO: 103 - DNA, 22 bp, Artificial Sequence
  synthetic oligonucleotide
agtgcaatggaacactcaagac

SEQ ID NO: 104 - DNA, 22 bp, Artificial Sequence
  synthetic oligonucleotide
cccaactccatgtttagactgg

SEQ ID NO: 105 - DNA, 20 bp, Artificial Sequence
  synthetic oligonucleotide
tgaccgttgtggacccaatg

SEQ ID NO: 106 - DNA, 24 bp, Artificial Sequence
  synthetic oligonucleotide
gaagttgcattcctcatacacatg

SEQ ID NO: 107 - DNA, 24 bp, Artificial Sequence
  synthetic oligonucleotide
gagagttttgactgtaataggaac

SEQ ID NO: 108 - DNA, 22 bp, Artificial Sequence
  synthetic oligonucleotide
tgtcacacagggagagataact

SEQ ID NO: 109 - DNA, 24 bp, Artificial Sequence
  synthetic oligonucleotide
ttagtaaccagattttgccatctg

SEQ ID NO: 110 - DNA, 24 bp, Artificial Sequence
  synthetic oligonucleotide
cccactctcaactttacatgatac

SEQ ID NO: 111 - DNA, 24 bp, Artificial Sequence
  synthetic oligonucleotide
cttctacagaatttgagggaaagt

SEQ ID NO: 112 - DNA, 23 bp, Artificial Sequence
  synthetic oligonucleotide
tccgtcgaactagaaatgcttag

SEQ ID NO: 113 - DNA, 20 bp, Artificial Sequence
  synthetic oligonucleotide
ccacctcaggatagttccag

SEQ ID NO: 114 - DNA, 22 bp, Artificial Sequence
  synthetic oligonucleotide
cctctgaatggttaggtcctta

SEQ ID NO: 115 - DNA, 20 bp, Artificial Sequence
  synthetic oligonucleotide
ctgggcgttagcaagtgatc

SEQ ID NO: 116 - DNA, 22 bp, Artificial Sequence
  synthetic oligonucleotide
cccacagtttaaacgctctgta

SEQ ID NO: 117 - DNA, 20 bp, Artificial Sequence
  synthetic oligonucleotide
atgccacccagaggatgttc

SEQ ID NO: 118 - DNA, 20 bp, Artificial Sequence
  synthetic oligonucleotide
gatggtcaggtctgcctttg

SEQ ID NO: 119 - DNA, 20 bp, Artificial Sequence
  synthetic oligonucleotide
gggttttggtggtggctttg

SEQ ID NO: 120 - DNA, 23 bp, Artificial Sequence
  synthetic oligonucleotide
tcgtggttcttcttcaggtaggc

SEQ ID NO: 121 - DNA, 22 bp, Artificial Sequence
  synthetic oligonucleotide
cacagcccaagatagttaagtg

SEQ ID NO: 122 - DNA, 26 bp, Artificial Sequence
  synthetic oligonucleotide
gcataaagtgtaagtgtataagcata

SEQ ID NO: 123 - DNA, 19 bp, Artificial Sequence
  synthetic oligonucleotide
gtggaggagctgtacctgg

SEQ ID NO: 124 - DNA, 19 bp, Artificial Sequence
  synthetic oligonucleotide
ggcaatgaggccacaggct

SEQ ID NO: 125 - DNA, 21 bp, Artificial Sequence
  synthetic oligonucleotide
gacccagccctttgttttgac

SEQ ID NO: 126 - DNA, 22 bp, Artificial Sequence
  synthetic oligonucleotide
tgtggacgttgatggtattggt

SEQ ID NO: 127 - DNA, 23 bp, Artificial Sequence
  synthetic oligonucleotide
gccccactcggatacttcttact

SEQ ID NO: 128 - DNA, 21 bp, Artificial Sequence
  synthetic oligonucleotide
gaggaatactgtggcccaaca

SEQ ID NO: 129 - DNA, 20 bp, Artificial Sequence
  synthetic oligonucleotide
ccagagcaggactggaaatg

SEQ ID NO: 130 - DNA, 20 bp, Artificial Sequence
  synthetic oligonucleotide
acgttgtgcagctggtagtc

SEQ ID NO: 131 - protein, 7 aa, Artificial Sequence
  synthetic peptide
RLLLRYL

SEQ ID NO: 132 - protein, 7 aa, Artificial Sequence
  synthetic peptide
RLLRRYL

SEQ ID NO: 133 - protein, 15 aa, Artificial Sequence
  synthetic peptide
MSWPRRLLLRYLFPA

SEQ ID NO: 134 - protein, 15 aa, Artificial Sequence
  synthetic peptide
MSWPRRLLRRYLFPA

SEQ ID NO: 135 - protein, 515 aa, Equus caballus
MSCLCRLLLTYLFPALLLHGLGEGSALLHPDSRSHPRSLEKNAWRAFKESQCHHMLKHLHNGARITVQMPPAIEGHWVSTGCEVRSGPEFLTRSYRFYHNNTFKAYQFYYGGNRCTSPTYTLVIRGKIRLRQASWIIRGGTEADYQLHHVQVICHTEAVAERLGQLVNHTCPGFVPTGGAWVRDVPYDLWREENGCECTRAVNFAMHELQLIRVEKQHLQHNLDHLVEELFLGDIHTDASQRVFYRPSSYQPPLQNAKNHDHACIACRIIHRSDEHRPPLLPPKADLTVGLHGEWVSQRCEVRPEVLFLTRHFIFHDNNNTWEGHYYHYSDPVCKHPTFTIYAKGRYSRGVHSSRVMGGTEFVFKVTHMKVTPMDAATASLLNVFNGNECGAEGSWQVGVQQDVTHTNGCVALGIKLPHTEYEIFKMEQDARGRYLLFNGQRPSDGSSPDRPEKRATSYQMPLVQCASSAPRAEDFSDDHRAHPSGSHAPRRSACPLLFAALSCLLTLPPWNLLR

SEQ ID NO: 136 - protein, 515 aa, Canis familiaris
MSGPSRLLLAYLSAALLLHGLGEGSALLHPDSRSHPRSLEKSAWRAFKESQCHHMLKHLHNGARITVQMPPAIEGHWVSTGCEVRSGPEFITRSYRFYHNNTFKAYQFYYGGNRCTSPTYTLVIRGKVRLRQASWIIRGGTEADYQLHNVQVVCHTEAVAERLSQRVNRTCPGFVPAGGPWLQDVPYDLWRAENGCECTKAVNFAMHELQLIRVEKQYLQHNLDRLVEELFLGDIHTDASQRMFYRPSSYQPPLQNAKNHDHACIACRIIHRSDEHRPPLLPPKADLTVGLHGEWVSQRCEVRPEVLFLTRHFIFHDNNNTWEGHYYHYSDPVCKHPTFTIYAKGRYSRGVHSSRVMGGTEFVFKVNHMKVTPMDAATASLLNVFNGNECGAEGSWQVGVQQDVTHTNGCVALGIKLPHTEYEIFKMEQDARGRYLLFNGQRPSDGSSPDRPEKRATSYQMPLVQCASSVPRAEDFSENYRGHQYGSWAPGRSACPLLLAVLPGLLTLLHWNVLR

SEQ ID NO: 137 - protein, 514 aa, Mus musculus
MSRVRRLLLGYLFPALLLHGLGEGSALLHPDSRSHPRSLEKSAWRAFKESQCHHMLKHLHNGARITVQMPPTIEGHWVSTGCEVRSGPEFMTRSYRFYNNNTFKAYQFYYGSNRCTNPTYTLIIRGKIRLRQASWIIRGGTEADYQLHGVQVICHTEAVAEQLSRLVNRTCPGFLAPGGPWVQDVAYDLWQEESNHECTKAVNFAMHELQLIRVEKQYPHHSLDHLVEELFLGDIHTDATQRVFYRPSSYQPPLQNAKNHNHACIACRIIFRSDEHHPPILPPKADLTIGLHGEWVSQRCEVRPEVLFLTRHFIFHDNNNTWEGHYYHYSDPVCKHPTFTIYARGRYSRGVLSSKVMGGTEFVFKVNHMKVTPMDAATASLLNVFSGNECGAEGSWQVGIQQDVTHTNGCVALGIKLPHTEYEIFKMEQDTRGRYLLFNGQRPSDGSSPDRPEKRATSYQMPLVQCASSSPRAEELLEDSQGHLYGRAAGRTAGSLLLPAFVSLWTLPHWRILR

SEQ ID NO: 138 - protein, 517 aa, Myotis lucifugus
MSCPCRLLLTYLFPALLLHGLGEGSALLHPDSRSHPRSLEKSAWRAFKESQCHHMLKHLHNGARITVQMPPAIEGHWVSTGCEVRSGPEFITRSYRFYHNNTFKAYQFYYGGNRCTSPTYTLIIRGKIRLRQASWIIRGGTEADYQLHSVQVISHTEAVAERLSQLVNRTCPGFVPAGGAWVQDVPYDLLQEEDGCECTRAVNFAMHELQLLRVEKQYLQHHLDRLVEELFLGDIHTDAAQRTFYRPSSYQPPLQNAKNHDHACIACRIIHRSDEHHPPLLPPKADLTVGLHGEWVSQRCEVRPEVLFLTRHFIFHDNNNTWEGHYYHYSDPVCKHPTFTIYAKGRYSRGVHSSRVMGGTEFVFKVNHMKVTPMDAATTSLLNVFNGNECGAEGSWQVGVQQDVTHTNGCVALGIKLPHTEYEIFKMEQDARGRYLLFNGQRPSDGSSPDRPEKRATSYQMPLVQCASSVSRPEDFSFSEDHRAQQYRSRAPGRSACPRRLAALACLLTLLHWNSLR

SEQ ID NO: 139 - protein, 515 aa, Gallus gallus
MFVPYGSLMRYLFPALLLHGLGEGSALLHPDSRSHPRSLEKSAWRAFKESQCHHMLKHLHNGARITVQMPPNIEGHWVSTGCEVRAGPEFITRSYRFYHNNTFKAYQFYYGGNRCTNPIYTLVIRGKIRLRQASWIIRGGTEADYQLRNIQIISHNEAVAKKLSELVNNTCPGFIPEDSPWEQDVSYDLLREENGCECTKALNFAMHELQLIRVEKQYLHHNLDHLVEELFLGDIHTDATQRRYYRPSSYQPPLQNAKNHDRTCIACRIIYRSDEHHPPILPPKADLTIGLHGEWVSQRCEVRPEVLFLTRHFIFHDNNNTWEGHYYHYSDPICKHPTFTIYAKGRYSRGVHSAKVMGGTEFVFKVNHMKVTPMDVSTASLLNVFNGNECGAQGSWQVGVQQDVTHTNGCIALGIRLPHTEYEIFKMEQDARGRYLLYNGQRPSDGSSPDRPEKRATSYQMPLIQCASSVPRSEESPEENKIRLYSSRAPAKHPSASALALVLFICTVSYWDILS

SEQ ID NO: 140 - protein, 515 aa, Pelodiscus sinensis
MSLPCCGRVRYLLLALLLHGLGEGSALLHPDSRSHPRSLEKSAWRAFKESQCHHMLKHLHNGARITVQMPPSIEGHWVSTGCEVRSGPEFITRSYRFYHNNTFKAYQFYYGGNRCTNPTYTLVIRGKIRLRQASWIIRGGTEADYQLHNVQIISHSEAVAEKLSQLVNRTCPGFVPGNSPWEQDVSYNLWREENGCECTRALNFAMHELQLIRVEKQYLHHNLDHLVEELFLGDIHTDATQRMYYRPSSYQPPLQNAKNHDHSCIACRIIYRSDEHHPPILPPKADLTIGLHGEWVSQRCEVRPEVLFLTRHFIFHDNNNTWEGHYYHYSDPICKHPTFTIYAKGRYSRGVHSSKVMGGTEFVFKVNHMKVTPMDIATASLLNVFNGNECGAEGSWQVGVQQDVTHTNGCVALGIRLPHTEYEIFKMEQNARGSYLLYNGQRPSDGSSPDRPEKRATSYQMPLVQCASSAPRPEESAEENKIGRYSSRAPKKDSSVFMLTLMLFICTGSHWNIHS

SEQ ID NO: 141 - protein, 515 aa, Xenopus tropicalis
MGIPCFYSSLISCLFSIITLVDVGQSSALTLSDSRLHPQSLEKSPWREFQCQHMLKHLHNGARVTVQMPPNIEGHWVSMGCEVRSGPEFITRSYRFYNNNTFKAYQHYYGNNHCTIPTYTLVIRGKIRLRQASWIIRGGTEADYQLHNVQIIPHSETVAEKLTWLVNHTCAGFVPGDMPWEPGISYDLWREEGGFKCTKALNFAMHELQLIRVEKQYMHHNLDHLVEELFLGDIHTDPSQRMYYRPSSYQPPLQNAKNHNQNCVACRIILRSDEHHPPILPAKADLPVGLNGEWVSQRCEVRPEVLFLTRHFIFNDNNHTWEGFYYHYSDPICKHPSFTIYAKGRYSRGVYSSKVMGGTEFVFKVNHMKVTPMDFATASLLNVFNGDECGAEGSWKVGVEQDVTHTNGCVALGIKLPHTEYELFRMEQDNRGRYLLYNGQRPSDGSSPARPEKRATSYQVPLVQCTSVSLNPEGAHDGQHKSQSRNSAAGHIFLYLFSNLFLLFICTLLHLEILS

SEQ ID NO: 142 - protein, 412 aa, Danio rerio
KDTECVQMLKHLHSGARITVQMPPDIQGHWVSTSCEVRPGPEFITRSYRFLHNSSFSALQFYYSDVHCSSPTHTLLVRGRLRLRQASWIVRGGTEADYQIQSTRVVFHSESAAAALGLQLERGCRSISRSWRLNMSYELWSEEKDSECTAQINFSMHELQLLRLEKQHLEPALEELYLGDIHTQAEHRLTYRPSSYQPALHNARNHDGRCLVCRMVSRSDEFHPPVLPVLPVLPVVLQGQWASRRCEVRPEVQFLTRHFIFHDSNSSWEGHYYHYSDPACRHPTFSITARGAYSRGAESARISGATEYIFVLPLDVSTVSLLNLSPGAQCGVRGSWRLGEQQDVTATQGCAALGIRLPHTEFEIFRSQRLQDGRLLLFNGQRPSDGSSPDRPERRPSSFQQPLMQCTEQSVS

SEQ ID NO: 143 - protein, 357 aa, Ciona intestinalis
MKKHHRLLVYSSQTNCDCTDELDFYLGELEVMKVVRVKDYRPPFKTVTQLFAGDVKTKIDNRRYYRSNTFQAPLERLGIMSSNIEMQIGRFFPPNLHRTLISPNSQQFILHGNWLSVRCELRPHSSFLTRHLFFPRNGNVWSGVYRYYVDDQCREPYYTLRAHGRYDRTAINPTNGDADYVFDARRMFITSHSVNLTSLLNDAKRGSLKLETHVEYDVPETEGIPLIDLSLPRREYDHIMMRRIDGNLLLFTGRRPNDGTIPDSADKRTTSYQDELLRCRSKCAFDDLRDSTFLCGISATELPGSIETTVIPNKEKHQHDFVAMVYQEATTSSAKKNMHSLTFILSLLCIIAILKYE

SEQ ID NO: 144 - protein, 26 aa, Homo sapiens
MSWPRRLLLRYLFPALLLHGLGEGSA

SEQ ID NO: 145 - protein, 26 aa, Equus caballus
MSCLCRLLLTYLFPALLLHGLGEGSA

SEQ ID NO: 146 - protein, 26 aa, Canis familiaris
MSGPSRLLLAYLSAALLLHGLGEGSA

SEQ ID NO: 147 - protein, 26 aa, Mus musculus
MSRVRRLLLGYLFPALLLHGLGEGSA

SEQ ID NO: 148 - protein, 26 aa, Myotis lucifugus
MSCPCRLLLTYLFPALLLHGLGEGSA

SEQ ID NO: 149 - protein, 26 aa, Gallus gallus
MFVPYGSLMRYLFPALLLHGLGEGSA

SEQ ID NO: 150 - protein, 26 aa, Pelodiscus sinensis
MSLPCCGRVRYLLLALLLHGLGEGSA

SEQ ID NO: 151 - protein, 28 aa, Xenopus laevis
MGIPCFYSSLISCLFSIITLVDVGQSSA

SEQ ID NO: 152 - protein, 68 aa, Artificial Sequence
  synthetic peptide
MSWPRRLLLRYLFPALLLHGLGEGSALLHPDSRSHPRSLEKSAWRAFKESQCHHMLKHNGARITVQMP

SEQ ID NO: 153 - protein, 68 aa, Artificial Sequence
  synthetic peptide
MSWPRRLLRRYLFPALLLHGLGEGSALLHPDSRSHPRSLEKSAWRAFKESQCHHMLKHNGARITVQMP

SEQ ID NO: 154 - protein, 20 aa, Artificial Sequence
  synthetic peptide
CQRPSDGSSPDRPEKRATSY

(19) United States
(12) Patent Application Publication
(10) Pub. No.: US 2011/0189097 A1
(43) Pub. Date: Aug. 4, 2011

(54) Use of WNT inhibitor to inhibit angiogenesis in the CNS

(76) Inventors: Dritan Agalliu, San Jose, CA (US); Richard Daneman, San Francisco, CA (US); Ben A. Barres, Palo Alto, CA (US)

(21) Appl. No.: 12/927,200

(22) Filed: Nov. 8, 2010

Related U.S. Application Data
(60) Provisional application No. 61/280,887, filed on Nov. 9, 2009.

(51) Int. Cl. A61K 38/17; C12N 5/071; C12N 5/09; A61P 25/00; A61K 38/02; A61K 31/7088; C07K 14/47; A61K 49/00; A61P 35/00; A61P 27/02

(52) U.S. Cl. 424/9.2; 435/375; 514/1.1; 514/17.7; 514/13.3; 514/44 R; 530/350

(57) ABSTRACT

Activation of the Wnt signaling pathway in cells is inhibited by contacting the cells with an Apcdd peptide. Wnt inhibition by this and other methods finds particular use in treating Wnt-mediated disease conditions such as conditions associated with aberrant angiogenesis in the CNS or aberrant cell proliferation.

### CROSS REFERENCE TO RELATED APPLICATIONS

Pursuant to 35 U.S.C. §119(e), this application claims priority to the filing date of the U.S. Provisional Patent Application Ser. No. 61/280,887 filed Nov. 9, 2009; the disclosure of which is herein incorporated by reference.

### FIELD OF THE INVENTION

Activation of the Wnt signaling pathway in cells is inhibited by contacting the cells with an Apcdd peptide. Wnt inhibition by this and other methods finds particular use in treating Wnt-mediated disease conditions, such as conditions associated with aberrant angiogenesis in the CNS or aberrant cell proliferation.

### BACKGROUND OF THE INVENTION

Wnt proteins form a family of highly conserved secreted signaling molecules. Insights into the mechanisms of Wnt action have emerged from several systems: genetics in Drosophila melanogaster and Caenorhabditis elegans; biochemistry in cell culture, ectopic gene expression in Xenopus laevis, and ectopic gene expression and gene mutation in mouse embryos. From these studies, it is currently understood that Wnt proteins exert their effects on cells by binding to receptors of the Frizzled and LRP families on the cell surface to activate intracellular signal cascades. In the best understood of these signal cascades (the canonical Wnt signaling pathway), cytoplasmic relay components mediate transduction of the signal to β-catenin, which then enters the nucleus and forms a complex with TCF to activate transcription of Wnt target genes.

Wnt signaling is involved in numerous biological processes in animal development, including the proliferation of stem cells, the specification of the neural crest, the differentiation of osteoblasts, and the induction of angiogenesis in the Central Nervous System (CNS). Additionally, Wnt signaling has been implicated in or postulated to play a role in disease processes that rely upon these same biological processes, for example, the aberrant expansion of cells including stem cells in cancer and the aberrant vascular endothelial cell growth in CNS tumors, diabetic retinopathy and age-related macular degeneration. Inhibitors of Wnt signaling are therefore potentially important reagents in the treatment of disease conditions in vivo. The development of pharmaceutically active Wnt inhibitor compositions is thus of great interest.

### Publications

The biological activity of soluble wingless protein is described in van Leeuwen et al. (1994) Nature 368(6469): 342-4. Biochemical characterization of Wnt-Frizzled interactions using a soluble, biologically active vertebrate Wnt protein is described by Hsieh et al. (1999) Proc Natl Acad Sci USA 96(7): 3546-51. The role of the Wnt signaling pathway in development and disease is described in Logan et al. (2004) Annu Rev Cell Dev Biol. 20: 781-810 and Westendorf et al. (2004) Gene 341: 19-29. The development of the vasculature of the Central Nervous System is described by Mancuso, M R et al. (2008) Lymphat Res Biol. 6(3-4): 173-180; Liebner, S. et al. (2008) J. Cell Biol. 183(3): 409-417; Polakis, P. (2008) J. Cell Biol. 183(3): 371-373; Stenman, J M et al. (2008) Science 322(5905): 1247-50; and Daneman, R. et al. (2009) PNAS (106)2: 641-646.

### SUMMARY OF THE INVENTION

Methods and compositions for antagonizing Wnt signaling in a cell are provided. These methods find particular use in inhibiting aberrant angiogenesis in the CNS or aberrant cell proliferation.

In one aspect of the invention, methods for inhibiting Wnt signaling in a cell by contacting the cell with an Apcdd polypeptide or a nucleic acid encoding an Apcdd polypeptide are provided. In some embodiments, the cell is in vitro. In certain embodiments, the cell is a cancer cell. In certain embodiments, the cell is an endothelial cell. In some embodiments, the cell is in vivo. In certain embodiments, the cell is in the CNS. In some embodiments, the Apcdd polypeptide is an Apcdd1 polypeptide. In some embodiments, the Apcdd polypeptide is an Apcdd1L polypeptide.

In one aspect of the invention, methods for inhibiting angiogenesis in a cell derived from the CNS by contacting the cell with an effect amount of a Wnt inhibitor are provided. In some embodiments, the Wnt inhibitor is a peptide, a nucleic acid, or a small molecule. In certain embodiments, the Wnt inhibitor is an Apcdd peptide. In certain embodiments, the Apcdd polypeptide is an Apcdd1 polypeptide. In certain embodiments, the Apcdd polypeptide is an Apcdd1L polypeptide. In some embodiments, the cell is in vitro. In certain embodiments, the cell is an endothelial cell. In some embodiments, the cell is in vivo. In some embodiments, the cell is an endothelial cell. In some embodiments, the cell is in a human suffering from a Wnt-mediated disorder. In certain embodiments, the disorder is diabetic retinopathy or age-related macular degeneration.

In another aspect of the invention, a composition that is an Apcdd polypeptide formulated for delivery to the CNS is provided.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1. The HHS phenotype maps on chromosome 18p11.2 in a point mutation in APCDD1 gene. a-d, Clinical appearance of HHS patients (a, b) and their hair shaft (c, d). Scale bar: 100 μm. e, Haplotype analysis of a Pakistani family HHS1. The linked haplotype is indicated in red. The critical recombination events are indicated by black arrowheads. f, In situ hybridization with APCDD1antisense mRNA probe in human hair follicles (HFs). APCDD1 is present in the dermal papilla (DP), matrix (Mx), hair shaft cortex (HSCx), and cuticle (HSCu) of the HF. g-j, Immunofluorescence in human HFs with a mouse polyclonal anti-APCDD1 antibody (Abnova). The expression of APCDD1 protein in the HSCx [white boxed in (g)] overlaps with that of E- and P-cadherins (h-j). Counterstaining with DAPI is shown in blue (g, j). Scale bars: 100 μm (f, g), 20 μm (h). k, Schematic of APCDD1 protein and the position of L9R mutation. l, A western blot with the mouse polyclonal anti-APCDD1 antibody (Abnova) from cell lysates of human scalp skin shows two fragments around 58 and 130 KDa in size. A similar pattern was observed with the HA-tagged wild-type APCDD1 overexpressed in HEK293T cells.

FIG. 2. Wild-type, but not L9R mutant APCDD1, inhibits canonical Wnt signaling. a, Co-immunoprecipitation assays show that extracellular domain of APCDD1 (APCDD1ΔTM) strongly interacts with extracellular domain of LRP5 (LRP5-EC) and WNT3A. Extracellular domain of a non-Wnt related single transmembrane receptor CD40 (CD40-EC) was used as a negative control. b, TOP/FOP Flash reporter assays in HEK293T cells. c, Effect of APCDD1 overexpression on transcriptional activity of the Wnt-specific Sia reporter gene induced by Wnt8 or β-catenin in Xenopus. APCDD1 (1 ng RNA) inhibited Wnt8-(50 pg RNA), but not β-catenin-induced (1 ng RNA) transcription. The number above the column indicates fold repression by APCDD1. d, The L9R mutant APCDD1 has a dominant negative effect on Wt APCDD1 in Xenopus. Activity of the Sia reporter gene induced by Wnt8 RNA (50 pg) was inhibited by coinjection of Wt APCDD1 RNA (1 ng), but not by coexpression of Wt and the L9R mutant. e, Western blot of the levels of APCDD1 in Xenopus embryos. L9R APCDD1 affects WT protein levels in Xenopus embryos. β-Gal is used as loading control. f, Western blot analysis of lysates from HEK293T cells transfected with Wt-, L9R- or L9V-APCDD1 1. g, The expression level of HA-tagged Wt-APCDD1 (Wt-HA) is decreased by co-expression with the L9R-APCDD1. β-actin was used as a normalization control (f, g). h-j, Immunofluorescence in transfected HEK293T with an APCCD1 antibody (green). Cell membrane was labeled with rhodamine phalloidin (red). Scale bar: 20 μm. k, Quantification of subcellular localization of APCDD1 isoforms. Bars represent mean±1 SEM (n=20 cells per group). Wt versus L9R, P<8×10−7; Wt versus Wt+L9R, P<3×10−6; L9R versus Wt+L9R, P=0.99. All P-values reported have the Bonferroni correction.

FIG. 3. Overexpression of Wt-APCDD1, but not L9R mutant, inhibits progenitor proliferation and neuronal specification in the chick spinal cord. Transfection of Wt-, L9R- or both APCDD1 isoforms is visualized by eGFP fluorescence (b, g, l; green) and in situ for hAPCDD1 (a, f, k). Sox3 labels neural progenitors in red and Tuj1 neurons in blue (b, g, l). Misexpression of Wt, but not L9R or a combination of both APCDD1 isoforms, reduces the number of Sox3+ neural progenitors. Immunofluorescence for Isl1/2+ (d, i, n; motor neurons MNs—ventral Isl1/2+ cells; dl3 interneurons—dorsal Isl1/2+ cells) shows that MNs are only reduced in the Wt APCDD1 electroporation. The D3 interneuron population is reduced in all conditions. Plots of Sox3+ progenitors (c, h, m) and Isl1/2+ MNs (e, j, o) in control (open circles) and transfected embryos (closed circles). Bars represent mean±s.e.m. (n=40 embryos for WT-APCDD1, n=35 embryos for L9R-APCDD1 and n=28 embryos for WT- and L9R-APCDD1, Student's t-test, *p<0.05).

FIG. 4. APCDD1 inhibits the Wnt pathway in Xenopus embryos. a, b, Dorsal overexpression of human APCDD1 (hAPCDD1) reduces axial and anterior structures (b). Scale bar: 4 mm (a). c-d, Ventral overexpression of hAPCDD1 produces a secondary axis (arrow in c) in cell autonomous fashion (GFP tracer, arrow in d). e-g, Phenotype of Xapcdd1 protein depletion and its rescue. Dorsal depletion by morpholino oligonucleotide (MO) produced a ventralized phenotype (e), rescued by Xapcdd1 RNA (f) and dominant-negative Wnt8 RNA (DN8) (g). h, APCDD1 is required in the signal-receiving cells. Wnt8 RNA and Siamois reporter gene were injected in adjacent cells. hAPCDD1 inhibited signaling only when coinjected with the reporter. i, Potential mechanism of action for wild-type and mutant APCDD1. Upper panel: WT APCDD1 (L) is processed in the ER and localized at the cell membrane, where it may inhibit Wnt signaling by interacting with WNT and LRP proteins. Lower panel: when WT and L9R-APCDD1 (R) are coexpressed, wild type APCDD1 is retained and degraded in the ER with the mutant, thus releasing Wnt signaling activity.

FIG. 5. Clinical appearance of Pakistani families with HHS, and identification of the mutation L9R in the APCDD1 gene in the families HHS1 and HHS2. a-l, Clinical features of HHS. Affected individuals in the families HHS1 (a-g, k, l) and HHS2 (h-j). The age of each individual is 2 (a), 6 (b), 10 (c), 3 (d), 28 (e), 20 (f), 28 (g), 8 (h), 9 (i), 12 (j), and 20 (k) years old, respectively. Hair shafts of affected individuals show tapered distal ends (l). Scale bar: 100 μm (l). m, Results of autozygosity mapping. The maximum LOD score was obtained for a region on chromosome 18 (m). n, Schematic representation of the candidate region harboring the HHS gene. o, Heterozygous mutation 26T>G (L9R) in APCDD1 gene of both families HHS1 and HHS2. (wild type allele: RLLLRYL, SEQ ID NO:131; mutant allele: RLLRRYL, SEQ ID NO:132) p, Results of haplotype analysis and screening assays for the mutation. The disease-related haplotype and affected individuals are colored in red (p). The PCR product from wild-type allele, 191 bp in size, was digested into 149 bp and 42 bp fragments, while that from the mutant allele was undigested (p). The 42 bp fragment was not shown. MWM, molecular weight markers; C, control individual (p).

FIG. 6. An Italian family with HHS. a, Pedigree of the family. b,c, Clinical appearance of affected individuals. Scale bars: 100 μm. d, Candidate region for the Italian family that was previously defined. The region found in Pakistani families, as well as the position of APCDD1 gene, are also shown. e, Identification of the heterozygous 26T>G (L9R) mutation in the APCDD1 gene in the Italian family. (wild type allele: MSWPRRLLLRYLFPA, SEQ ID NO:133; mutant allele: MSWPRRLLRRYLFPA, SEQ ID NO:134). f, Comparison of haplotypes between three families with an identical point mutation in the APCDD1 gene. The marker APCDD1-MS is located within intron 1 of the APCDD1 gene, which is only 5 Kb distant from the position of the mutation. Note that the three families had a distinct disease-related haplotype, suggesting that the mutation arose independently in each family, and that nucleotide 26 of the APCDD1 gene may be a mutational hotspot.

FIG. 7. APCDD1 mRNA is expressed in human scalp skin. a, RT-PCR amplification of APCDD1 mRNA from human scalp skin. Note that APCDD1 mRNA was amplified, while its homologue APCDD1L mRNA was not. b, RT-PCR using total RNA from human plucked hairs shows the expression of LRP5 and WNT3A in human hair follicles. MWM, molecular weight markers (a, b).

FIG. 8. Multiple amino acid sequence alignment of APCDD1 proteins between different species, and prediction of the signal peptide for APCDD1 protein. a, Alignment of the full APCDD1 protein from humans (Homo sapiens, Genbank Accession No. NP_{—}694545, SEQ ID NO:2), horse (Equus caballus, Ensembl Accession No. ENSECAP00000009668, SEQ ID NO:135), dog (Canis familiaris, Genbank Accession No. XP_{—}537333, SEQ ID NO:136), mouse (Mus musculus, Genbank Accession No. NP_{—}573500, SEQ ID NO:137), bat (Myotis lucifugus, Ensembl Accession No. ENSMLUP00000001735, SEQ ID NO:138), chicken (Gallus gallus, Ensembl Accession No. ENSGALP00000001313, SEQ ID NO:139), turtle (Pelodiscus sinensis, Genbank Accession No. BAD74115, SEQ ID NO:140), frog (Xenopus tropicalis, Genbank Accession No. ENSXETP00000056413, SEQ ID NO:141), zebrafish (Danio rerio, Ensembl Accession No. ENSDARP00000081410, SEQ ID NO:142), and sea squirt (Ciona intestinalis, Ensembl Accession No. ENSCINP00000022338, SEQ ID NO:143). The N-terminal signal peptide and the C-terminal transmembrane sequences are boxed in red and black, respectively. The conserved residues among 6 species are indicated by asterisks. The amino acid residue L9 is indicated in blue. Highly conserved cysteine residues are highlighted in yellow. b, Multiple amino-acid sequence alignment of the APCDD1 signal peptide sequences between human (Homo sapiens, SEQ ID NO:144), horse (Equus caballus, SEQ ID NO:145), dog (Canis familiaris, SEQ ID NO:146), mouse (Mus musculus, SEQ ID NO:147), bat (Myotis lucifugus, SEQ ID NO:148), chicken (Gallus gallus, SEQ ID NO:149), turtle (Pelodiscus sinensis, SEQ ID NO:150), and frog (Xenopus laevis, SEQ ID NO:151). Residues that are conserved among at least five species are colored yellow. The L9 is colored in red. c, d, The N-terminal signal peptide sequences of the wild type (c) (SEQ ID NO:152) and the L9R mutant (d) (SEQ ID NO:153) APCDD1 protein was analyzed using the SignalP-HMM program. The predicted hydrophobic core sequences are boxed. Amino acid position 9 is indicated by red arrowheads. Note that several other autosomal dominant diseases, such as familial hypocalciuric hypercalcemia (OMIM 145980) and antithrombin III deficiency (OMIM 107300 are also caused by substitutions in a leucine residue within the signal peptide.

FIG. 9. APCDD1 is able to interact with LRP5 and WNT3A in vitro. a, Co-immunoprecipitation assays in HEK293T cells. The HA-tagged extracellular domain of APCDD1 protein (APCDD1-ΔTM-HA) was co-immunoprecipitated with the Flag-tagged extracellular domain of LRP5 (LRP5-EC-Flag; left panel). Flag-tagged extracellular domain of APCDD1 protein (APCDD1-ΔTM-Flag) was co-immunoprecipitated with the HA-tagged WNT3A (WNT3A-HA), but not with the HA-tagged extracellular domain of CD40 (CD40-EC-HA; right panel). b, GST-pulldown assays. N-terminal GST fusion protein for the extracellular domain of APCDD1 (GST-APCDD1-ΔTM) was generated in bacteria, and was purified with glutathione-Sepharose beads (left panel). The purified GST-APCDD1-ΔTM was incubated with lysates of HEK293T cells overexpressing LRP5-EC-Flag, WNT3A-HA, or CD40-EC-HA, and was analyzed by western blots with mouse monoclonal anti-Flag-M2 (1:1,000; Sigma) or rabbit polyclonal anti-HA (1:4,000; Abcam) antibodies. The GST-APCDD1-ΔTM showed an affinity with LRP5-EC-Flag and WNT3A-HA, but not with CD40-EC-HA (right panels). CD40 is a Wnt signaling-unrelated single-pass transmembrane protein, and was used as a negative control (a, b).

FIG. 10. Overexpression of Wt-APCDD1, but not L9R mutant, blocks the activation of the Wnt reporter TOP::eGFP in the chick spinal cord. a-f Immunostaining for eGFP and APCDD1 in chick neural tubes electroporated with the Wnt reporter TOP::eGFP alone (a), or together with Wt (c), or L9R (e) APCDD1. Note that high eGFP levels in the dorsal and intermediate spinal cord from the Wnt reporter were inhibited strongly by the Wt APCDD1 and very weakly by L9R. This effect is illustrated in the diagrams (b, d, f).

FIG. 11. Overexpression of WT- or L9V-, but not L9R-APCDD1 inhibits progenitor proliferation and generation of neurons in the chick spinal cord. Immunofluorescence for Chx110+ (V2a ventral interneurons; a, e, i), or in situ hybridization for Sim1+ (c, g, k; V3 interneurons) shows that these neuronal populations are only reduced in the Wt APCDD1, but not L9R or both isoform electroporation. Plots of Chx10+ V2a interneurons (b, f, j) and Isl1/2+ dl3 interneurons (d, h, l) in control (open circles) and transfected embryos (closed circles) show that V2a interneurons are reduced in WT-APCDD1 transfection. Transfection of L9V APCDD1 is revealed by in situ hybridization for human APCDD1 (m) and eGFP immunofluorescence (n, green). Immunofluorescence for Sox3 labels neural progenitors in red and Tuj1 labels neurons in blue (n). Note that misexpression of L9V APCDD1 reduces the number of Sox3+ progenitors and neurons. The inhibition of progenitor proliferation was more effective in the ventral than dorsal spinal cord, because of lower Wnt/β-catenin levels in this region. Immunofluorescence for Chx10+ (V2a ventral interneurons; r), Isl1/2+ (p; MNs—ventral Isl1/2+ cells; dl3 interneurons—dorsal Isl1/2+ cells) or in situ for Sim+ (t; V3 interneurons) shows that these neurons are reduced in the L9V APCDD1 electroporation. Plots of Sox3+ progenitor numbers (o), Chx10+ V2a interneuron number (s), Isl1/2+ MN number (q) or Isl1/2+ dl3 interneurons (u) in control (open circles) and transfected embryos (closed circles). Bars represent mean±s.e.m. (n=40 embryos for WT-APCDD1, n=35 embryos for L9R-APCDD1, n=28 embryos for WT- and L9R-APCDD1, and n=15 embryos for L9V-APCDD1, Student's t-test, *p<0.05).

FIG. 12. Overexpression of mouse Apcdd1 or Apcdd1ΔTM inhibits progenitor proliferation and generation of neurons in the chick spinal cord. Transfection of mouse Apcdd1 or Apcdd1ΔTM is revealed by immunofluorescence for eGFP (b, k; green) and in situ hybridization for mApcdd1 (a, j). Sox3 labels neural progenitors in red and Tuj1 labels neurons in blue (b, k). Note that misexpression of either mApcdd1 or mApcdd1ΔTM, reduces by 2-fold the number of Sox3+ neural progenitors and neurons. The reduction in progenitor proliferation was more pronounced in the ventral than dorsal spinal cord, with lower levels of the Wnt/β-catenin signaling. Immunofluorescence for Chx10+ (V2a ventral interneurons; d, m), Isl1/2+ (f, o; MNs—ventral Isl1/2+ cells; dl3 interneurons—dorsal Isl1/2+ cells) or in situ for Sim1+ (h, q; V3 interneurons) reveals that these neuronal populations are also reduced. Plots of Sox3+ progenitor numbers (c, l), Chx10+ V2a interneuron number (e, n), Isl1/2+ MN number (g, p) or Isl1/2+ dl3 interneurons (i, r) in control (open circles) and transfected embryos (closed circles). Bars represent mean±s.e.m. (n=20 embryos transfected with mApcdd1; n=16 embryos transfected with mApcdd1ΔTM; Student's t-test; **p<0.01). Note the similarity in the inhibition of neural progenitor number and ventral and dorsal interneuron and motor neurons between mApcdd1 and mApcdd1ΔTM.

FIG. 13. Misexpression of either mouse Apcdd1 or mApcdd1ΔTm isoforms reduces the expression of several progenitor domain markers in the chick spinal cord. a-e, Immunohistochemistry for eGFP (a), Olig2 (b), Nkx2.2 (c), Pax 6 (d) and Pax7 (e) in mApcdd1-transfected chick spinal cord (stage 22). Note that misexpression of mApcdd1 in the ventral spinal cord reduces the domain of Olig2 (pMN) and to a much lesser extent that of Nkx2.2 (p3). In addition it slightly represses the expression of Pax6 (d), but not Pax7 (e). f-j, Immunohistochemistry for eGFP (f), Olig2 (g), Nkx2.2 (h), Pax 6 (i) and Pax7 (j) in mApcdd1ΔTM-transfected chick spinal cord (stage 22). Note that mApcdd1ΔTM also represses the expression of Olig2 (pMN) and to a lesser degree that of Nkx2.2 (p3) in the ventral spinal cord.

FIG. 14. Characterization of the APCDD1 protein. a, Cell lysates from HA-tagged wild-type APCDD1-expressing HEK293T cells were treated with N-glycosydase (PNGase F). The 68 KDa fragment was clearly digested into a 53 KDa fragment with PNGase F, suggesting that the APCDD1 protein undergoes N-glycosylation. b, Equal amounts of the cell lysate from HA-tagged wild-type APCDD1-expressing HEK293T cells were separated by 10% SDS PAGE under either non-reducing (−) or reducing (+) conditions. The intensity of the 130 KDa fragment markedly increased under non-reducing conditions. c, Co-immunoprecipitation (Co-IP) assays between Flag-tagged APCDD1 (APCDD1-Flag) and HA-tagged APCDD1 (APCDD1-HA) proteins. APCDD1-Flag protein is co-immunoprecipitated with APCDD1-HA protein (left panel), and APCDD1-HA protein is co-immunoprecipitated with APCDD1-Flag protein (right panel). These results demonstrate homodimerization of the APCDD1 protein. d, We transfected HEK293T cells with a full-length APCDD1 expression construct containing a Flag-tag just downstream of the signal peptide and an HA-tag at the C-terminus, and analyzed cell lysates and supernatants by western blotting. An expression construct for a truncated APCDD1 lacking the transmembrane domain (APCDD1-ΔTM) was also transfected as a positive control. S: signal peptide. TM: transmembrane domain. Western blots with anti-Flag, anti-APCDD1 and anti-Flag antibodies detected a strong fragment, around 63 KDa in size, in the medium of APCDD1-ΔTM-expressing cells, while no fragments were detected in medium of full-length APCDD1-expressing cells. β-actin was used as a normalization control for the cell lysate, and also used to show that the cell lysate did not contaminate the medium.

FIG. 15. The mutation L9R affects the co-translational processing of the mutant APCDD1. a-k, Immunofluorescence for APCDD1 on HEK293T cells (a, b, i-k) or Bend3.0 cells (c-h) transfected with Wt-(a, c, f, i), L9R-(b, d, g, j), L9V-(e, h), or co-transfected with Wt- and L9R-APCDD1 (k). Cell membrane was labeled with an anti-pan-cadherin antibody (a, b), and ER was stained with an anti-calnexin antibody (i-k). Scale bar: 20 μm (a). Bend3.0 cells were either not permeabilized with TritonX-100 (c-e) to determine surface expression of APCDD1 or permeabilized (f-h) to detect total protein. Note that Wt- or L9V-APCDD1 localize to the plasma membrane (a, c, f, e, h, i), whereas the L9R-APCDD1 is retained in the ER (b, d, g, j). The majority of Wt-APCDD1 co-localizes with L9R-APCDD1 in the ER when co-transfected (k). l-n, N-terminal GFP-tagged APCDD1 proteins (GST-APCDD1) were overexpressed in HEK293T cells, and analyzed by western blot (l) and immunofluorescence (m, n) using a rabbit polyclonal anti-APCDD1 antibody. The western blot shows that GFP-Wt APCDD1 was cleaved, while GFP-L9R mutant was not (l). β-actin was used as a normalization control (l). GFP-Wt-APCDD1 protein is detected at the cell membrane (m), while the GFP-L9R-APCDD1 is retained in the cytoplasm (n). The bottom panels are merged images and counterstaining with DAPI is shown in blue (a, b, m, n). o-q, Analysis of the amount of APCDD1 protein in both (o), cell membrane (p) and intracellular (q) compartments from immunofluorescence in FIG. 2h-j. The expression of L9R-APCDD1 is significantly lower than that of Wt-APCDD1 (o). Note that co-transfection of. Wt- and L9R-APCDD1 (Wt+L9R) decreased the membrane localized-APCDD1 protein (p), while it increased the expression of APCDD1 protein inside the cells (q). Data are represented as average±1 SEM (n=20 cells per group). Wt versus L9R, P<3×10⁻⁹; Wt versus Wt+L9R, P=0.58; L9R versus Wt+L9R, P<3×10⁻⁶ (o). Wt versus L9R, P<3×10⁻⁹; Wt versus Wt+L9R, P<2×10⁻⁵; L9R versus Wt+L9R, P<3×10⁻⁵ (p). Wt versus L9R, P=0.99; Wt versus Wt+L9R, P<9×10⁻⁵; L9R versus Wt+L9R, P<3×10⁻⁴ (q). All P-values reported have the Bonferroni correction (o-q).

FIG. 16. Timing (a) and localization (b, for stage 10) of Xapcdd1 expression by RT-PCR. gsc is a dorsal (organizer) gene and ODC and EF-1α are loading controls. Left panel in b shows a schematic representation of the three layers of blastula stage Xenopus embryos. Ectoderm and neural tissue derives from animal cells, mesoderm from marginal cells, and endoderm from vegetal cells.

FIG. 17. Xenopus apcdd1 MO specifically inhibit translation of wild type protein. In vitro translation for Xenopus WT-apcdd1 and 5′mut-apcdd1-HA (1 μg RNA) on the presence and absence of Xapcdd1-MO (1 μg). The slight difference in molecular weight between wild type and 5′mut is due to the two HA tags in the mutant.

FIG. 18. Wnt signaling is activated specifically in brain endothelial cells. (A) GeneChip analysis of Wnt signaling components in purified endothelial cells. FACS analysis was used to purify endothelial cells from the brain, liver and lung of Tie2eGFP mice, and gene expression was analyzed using Affymetrix microarray analysis. The expression of several molecules that have been demonstrated to be downstream of Wnt/β catenin signaling are enriched in brain endothelial cells compared with the liver and lung samples. For each probe set values are normalized to brain endothelial cells sample. (B-E) Tissue sections from the cerebral cortex (B and C higher magnification), heart (D) and lung (E) of an E12.5 TOP-Gal transgenic mouse were stained with an anti-LacZ antibody to indicate Wnt activity (i), the vessel marker BSL and the nuclear stain DAPI (ii). In merged images (iii), yellow arrows point to co-localization of LacZ and BSL signals. Wnt activity is observed in blood vessels in the brain, but not heart or lung at E12.5. (Scale bar, 100 μm (B, D, E) and 50 μm (C)).

FIG. 19. Expression of Wnts in the developing mouse CNS. In situ hybridizations demonstrating Wnt ligand expression in the developing forebrain (A-H) and spinal cord (Ai-Hi) of E11.5 mice. Canonical Wnt ligands Writ7a and Wnt7b are expressed by neural progenitors in the ventricular zone in the ventral-lateral spinal cord and cortical forebrain, whereas canonical Wnt ligands Wnt1, Wnt3, and Wnt3a are expressed by neural progenitors in the ventricular zone of the dorsal spinal cord and the hindbrain. Non-canonical Wnt ligands Wnt4, Wnt5a, and Wnt5b are also expressed by neural progenitors located in spatially distinct regions of the spinal cord and cortex. Double fluorescent in situ hybridizations in the developing forebrain (E10.5 I-K, E11.5 L-N) and spinal cord (E10.5 Ii-Ki, E11.5. Li-Ni) with Wnt7b (I, L, Ii, Li) and Claudin-5 (Cldn5) (J, M, Ji, Mi) and merged (K, N, Ki, Ni) demonstrate that Cldn5 positive vessels vascularize Wnt7b-positive regions of the developing CNS.

FIG. 20. Conditional depletion of β-catenin in endothelial cells leads to CNS specific vascular defects. (A) Cross sections of E11.5 (ii) endothelial-specific β-catenin mutants (β-cat^{flox/flox}; Tek-cre) and (i) litter-mate controls were stained with the nuclear marker DAPI (blue) and an antibody against the vascular marker CD31 (red). Normal vasculature was observed in peripheral tissues of both genotypes, whereas, angiogenesis defects were observed in the CNS of the endothelial-specific β-catenin mutants. White boxes outline developing neural tube. (Scale bar, 500 μm.) (B and C) Cross-sections of developing neural tube of an E11.5 (C) endothelial-specific β-catenin mutant and (B) littermate taken along the rostral to caudal axis (i-iii), were stained with the nuclear marker DAPI (blue) and an antibody against the vascular marker CD31 (red). The CNS of the endothelial-specific β-catenin mutants demonstrated a decrease in vascular density, a loss of capillary beds and the presence of malformed vessels (white arrows). (Scale bar, 100 μm.) (D) Sagittal sections through the developing neural tube of an E11.5 (ii) endothelial-specific β-catenin mutant and (i) litter-mate were stained with the nuclear marker DAPI (blue) and the vascular marker BSL (green). Large aggregates of endothelial cells were observed in the endothelial-specific β-catenin mutants. (Scale bar, 100 μm.)

FIG. 21. Abnormal vasculature in the CNS of Wnt7 mutants. A-D) Coronal tissue sections of the E10:5 spinal cord in Wnt7a, Wnt7b double heterozygotes (A: Wnt7a^{+/-}; Wnt7b^{+/}), Wnt7a mutants (B: Wnt7a^{-/-}; Wnt7b^{+/-}), Wnt7b mutants (C: Wnt7a^{+/-}; Wnt7b^{-/-}), and Wnt7a, Wnt7b double mutants (D: Wnt7a^{-/-}; Wnt7b^{-/-}) were stained with the nuclear marker DAPI (Blue) and the vascular marker CD31 (red). Normal capillary beds were observed in the wild-type and Wnt7a mutants, whereas vascular malformations and thickened vascular plexus were observed in the Wnt7b mutants, and large vascular plexus dilations where observed in double mutants. Normal capillaries and normal vascular plexus are indicated with white and yellow arrows respectively, whereas vascular malformations and abnormal vascular plexus are indicated with white and yellow arrow heads respectively. (Scale bar, 100 microns).

FIG. 22. Wnt7a regulates CNS endothelial cell migration and expression of the BBB-specific transporter Glut-1 (Slc2a1). (A) Measurement of mouse brain endothelial cell (bEnd3.0) migration across a fibronectin-coated filter to a basal media, basal media containing VEGF, basal media containing Wnt, or basal media containing both VEGF and Wnt. Wnt7a is a potent migration factor for CNS endothelial cells. Error bars represent standard error of the mean. *<0.001 as analyzed by a two tailed standard T-test not assuming normal variance. (B) Table of GeneChip values, given in arbitrary units, for primary cultures of mouse brain endothelial cells grown in basal medium, or basal medium with Wnt7a. Wnt7a up-regulates expression of transcripts encoding molecular transporters (Slc2a1, Slc7a1, and Slc7a5) but not tight junction molecules (Occludin, ZO-1) or pan-endothelial cell adhesion molecules (PECAM, VE-Cadherin). (C-F) Cross sections of the developing CNS of E11.5 endothelial-specific β-catenin mutants (E and F) and littermate controls (C and D) were stained with vascular marker BSL I (i, green) and an antibody directed against the BBB-specific glucose transporter Glut-1 (ii, red). Glut-1 specifically stains vascular endothelial cells in the control animals, whereas Glut-1 does not stain the vascular endothelial cells in the endothelial-specific β-catenin mutants but instead stains. CNS parenchymal cells (iii, merged images). (Scale bar, 100 μm.)

FIG. 23. Expression of Frizzled receptors in CNS endothelial cells. (A) GeneChip analysis of Frizzled receptors in purified endothelial cells. FACS analysis was used to purify endothelial cells from the brain, liver, and lung of Tie2GFP mice, and gene expression was analyzed using Affymetrix microarray analysis. Expression of Fzd4, Fzd6, and Fzd8 was observed in CNS endothelial cells, with Fzd6 enriched in CNS endothelial cell compared with the liver and lung samples. (B) In situ hybridizations demonstrating Fzd6 expression in CNS endothelial cells in the E12.5 mouse forebrain and spinal cord. Fzd6 is expressed in the vascular cells (black arrows) in the neural tissue.

FIG. 24. Normal vasculature in non-neural tissue and hindbrain of endothelial-specific-catenin mutants. (A-D) Tissue sections of the developing limbs (A), liver (B), heart (C), and hindbrain (D) of E11.5 (ii) endothelial-specific β-catenin mutants and (i) litter mate controls were stained with the nuclear marker DAPI (blue); the vascular marker CD31 (red) demonstrating that β-catenin is not required for blood vessel formation in these tissues. (Scale bar, 100 microns).

FIG. 25. Cellular analysis of vascular malformations in endothelial specific β-catenin mutants. (A) Cross sections of the developing neural tubes, and (B) sagittal sections of the forebrain of E11.5 (ii) endothelial-specific β-catenin mutants and (i) litter-mate controls were stained with the nuclear marker DAPI (blue), the vascular marker BSL (green), and an antibody against the pericyte marker NG2 (red). Vascular malformations in the endothelial-specific β-catenin mutants consisted of aggregates of endothelial cells surrounded by pericytes. The aggregates could be found to form disorganized balls of cells with no discernible lumen (white arrow head in Bii) or layers of cells surrounding a lumen carrying blood (white arrow in Aii). (Scale bar, 100 microns.) (C-D) Cross sections of developing neural tubes of E11.5 (ii) endothelial specific β-catenin mutants and (i) litter-mate controls were stained with the vascular marker BSL (green) and anti-serum directed against PARD6 which stains blood cells (red). Vascular malformations in the β-catenin mutants were associated with small leakage of blood cells (yellow arrow in Cii) and large hemorrhages (Dii). (Scale bar, 100 microns).

FIG. 26. Expression of adherens junctions and tight junctions proteins in endothelial-specific β-catenin mutants. Sagital sections of the spinal cord of E11.5 endothelial-specific catenin mutants (B, D, F, and H) and litter-mate controls (A, C, E, and G) were stained with antibodies directed against VE-cadherin (A and B), a-catenin (C and D), ZO-1 (E and F), and Occludin (G and H) and double labeled with the vascular marker BSL (green in merge images A′-H′). Each adherens junction (VE-cadherin and a-catenin) and tight junction (ZO-1 and Occludin) component was present at cellular junctions in both normal vessels in the control animals and in the vascular malformations in the endothelial-specific β-catenin mutants. White arrows point to cell junctions. (Scale bar, 50 microns)

FIG. 27. Inhibition of Wnt signaling leads to CNS-specific vascular defects. (A-D) Pregnant female C57bl6 mice were administered intravenously an adenovirus encoding a (B) soluble Frizzled 8 receptor-FC fusion (Ad-sFz8-FC) or (A) a control FC (Ad-FC) at 9 days of gestation. Cross sections of E12.5 forebrain were stained with the nuclear stain DAPI (blue) and the vascular stain BSL (green). Normal tissue capillary beds were observed throughout the forebrain in embryos treated with Ad-FC (Ai and ii), whereas CNS vascular defects were observed in the forebrains of embryos treated with Ad-sFz8-FC (B). The tissue vasculature of Ad-sFz8-FC-treated animals ranged from normal (Bi), to small aggregates of endothelial cells (Bii), to large malformations that were stuck to the meningeal surface (Biii and iv). Apparently normal vasculature was found in non-neural tissues including the limbs (Ci and Di) and the liver (Cii and Dii) in animals treated with both viruses. (Scale bar, 100 microns.) (E) The vasculature of embryos treated with Ad-FC and Ad-sFz8-FC were quantified for percent forebrain vascular length associated with the meningeal surface. Ad-sFz8-FC treated embryos exhibited blood vessels with an increase in association with the meningeal surface. Error bars represent standard error of the mean. *<0.002 as analyzed by a two tailed standard T-test not assuming normal variance. (F) The vasculature of embryos treated with Ad-FC and Ad-sFz8-FC were quantified for percent vascular length in forebrain, liver, and limbs with abnormally thick vessels identified as vascular segments thicker than 3 cell bodies across. The values were graphed relative to the average of the Ad-FC. Ad-sFz8-FC treated embryos exhibited thicker blood vessels in the forebrain but not the liver or limbs. Error bars represent standard error of the mean. *<0.002 as analyzed by a two tailed standard T-test not assuming normal variance.

FIG. 28. Apcdd1mRNA is expressed in the vasculature of the developing Central Nervous System. A-I In situ hybridization with the Apcdd1 antisense mRNA probe in the developing embryonic (A-C) and postnatal (G-I) brain and embryonic spinal cord (D-F) at various stages of CNS development. Apcdd1 transcript is present in the vasculature of the developing CNS from the time when the vessels invade the CNS (E10.5-E11.0) through postnatal day 20 (P20) when the brain is fully mature in size, but is extinguished from adult (P60) vasculature. In addition, Apcdd1 is expressed in regions of the CNS where there is active canonical Wnt signaling such as the cortical hem at E11.5 (A), the deep layer cortical neurons (B, C), the dentate gyrus (I) and the spinal cord progenitors (D-F).

FIG. 29. Apcdd1 protein is expressed in the vasculature of the CNS but not of other organs. In situ hybridization with the CD31 (Pecam1; A,D), Claudin-5 (Cldn5; B, E) and Apcdd1 (C, F) antisense mRNA probes on neocortical (A-C) and liver (D-F) tissue at postnatal day 20 (P20). Note that Apcdd1 transcript is present in the CNS vasculature, but not liver vasculature, whereas the transcripts for Pecam1 and Claudin-5 are present in both organs.

FIG. 30. Apcdd1 protein is present in the Central Nervous System vasculature. A-F Immunofluorescence for Apcdd1 (A, D), the tight junction protein ZO-1 (B, E) and merge panels (C, F) in the postnatal day 20 brain. Note that Apcdd1 protein is present in blood vessels and other CNS cell types but it is not localized to tight junctions. G. Schematic diagram of the full length Apcdd1 protein and the extracellular domain (Apcdd1DTm). H. Western blots of Apcdd1, Claudin-5, Caveolin-1 and β-actin in HEK293 cells transfected with empty vector or isoforms of Apcdd1 and in brain lysates isolated from E11.5 through P8 brain and P8 liver. Note that Apcdd1 is present at all stages of development in the CNS but not liver, although the lower band is present at high levels at E17.5 and P8. Claudin-5 and Caveolin-1, two proteins that are present at tight junctions and caveolae, respectively, in endothelial cells, respectively, are expressed at similar levels. The total amount of the loaded protein is revealed by b-actin Western blots.

### DETAILED DESCRIPTION OF THE INVENTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

Methods and compositions for antagonizing Wnt protein signaling in a cell are provided. These methods find particular use in inhibiting aberrant angiogenesis in the CNS and aberrant cell proliferation. These and other objects, advantages, and features of the invention will become apparent to those persons skilled in the art upon reading the details of the compositions and methods as more fully described below.

Wnt signaling. A “Wnt protein” is a member of the family of highly conserved secreted signaling molecules which play key roles in both embryogenesis and mature tissues. The human Wnt gene family has at least 19 members: Wnt-1 (RefSeq.: NM_{—}005430), Wnt-2 (RefSeq.: NM_{—}003391), Wnt-2B (Wnt-13) (RefSeq.: NM_{—}004185), Wnt-3 (ReSeq.: NM_{—}030753), Wnt3a (RefSeq.: NM_{—}033131), Wnt-4 (RefSeq.: NM_{—}030761), Wnt-5A (RefSeq.: NM_{—}003392), Wnt-5B (RefSeq.: NM_{—}032642), Wnt-6 (RefSeq.: NM_{—}006522), Wnt-7A (RefSeq.: NM_{—}004625), Wnt-7B (RefSeq.: NM_{—}058238), Wnt-8A (RefSeq.: NM_{—}058244), Wnt-8B (RefSeq.: NM_{—}003393), Wnt-9A (Wnt-14) (RefSeq.: NM_{—}003395), Wnt-9B (Wnt-15) (RefSeq.: NM_{—}003396), Wnt-10A (RefSeq.: NM_{—}025216), Wnt-10B (RefSeq.: NM_{—}003394), Wnt-11 (RefSeq.: NM_{—}004626), Wnt-16 (RefSeq.: NM_{—}016087). Although each member has varying degrees of sequence identity with the family, all encode small (i.e., 39-46 kD), acylated, palmitoylated, secreted glycoproteins that contain 23-24 conserved cysteine residues whose spacing is highly conserved (McMahon, A P et al., Trends Genet. 1992; 8: 236-242; Miller, J R. Genome Biol. 2002; 3(1): 3001.1-3001.15).

“Wnt protein signaling” or “Wnt signaling” is used herein to refer to the mechanism by which Wnt proteins modulate cell activity. Wnt proteins modulate cell activity by binding to Wnt receptor complexes that include a polypeptide from the Frizzled (Fzd) family of proteins and a polypeptide of the low-density lipoprotein receptor (LDLR)-related protein (LRP) family of proteins. Fzd proteins are seven-pass transmembrane proteins (Ingham, P. W. (1996) Trends Genet. 12: 382-384; YangSnyder, J. et al. (1996) Curr. Biol. 6: 1302-1306; Bhanot, P. et al. (1996) Nature 382: 225-230). There are ten known members of the Fzd family (Fzd1 through Fzd10), any of which may be used in the Wnt receptor complex LRP proteins are single-pass transmembrane proteins that bind and internalize ligands in the process of receptor-mediated endocytosis; LRP family members LRP5 (RefSeq.: NM_{—}002335.2) or LRP6 (RefSeq.: NM_{—}002336.2) are included in the Wnt receptor complex.

Once activated by Wnt binding, the Wnt receptor complex will activate one or more intracellular signaling cascades. These include the canonical Wnt signaling pathway; the Wnt/planar cell polarity (Wnt/PCP) pathway; and the Wnt-calcium (Wnt/Ca²⁺) pathway (Giles, R H et al. (2003) Biochim Biophys Acta 1653, 1-24; Peifer, M. et al. (1994) Development 120: 369-380; Papkoff, J. et al (1996) Mol. Cell Biol. 16: 2128-2134; Veeman, M. T. et al. (2003) Dev. Cell 5: 367-377). For example, activation of the canonical Wnt signaling pathway results in the inhibition of phosphorylation of the intracellular protein β-catenin, leading to an accumulation of β-catenin in the cytosol and its subsequent translocation to the nucleus where it interacts with transcription factors, e.g. TCF/LEF, to activate target genes.

The phrases “Wnt-mediated condition” and “Wnt-mediated disorder” are used interchangeably herein to describe a condition, disorder, or disease state characterized by aberrant Wnt signaling. In a specific aspect, the aberrant Wnt signaling is a level of Wnt signaling in a cell or tissue suspected of being diseased that exceeds the level of Wnt signaling in a similar non-diseased cell or tissue. Examples of Wnt-mediated disorders include those associated with aberrant angiogenesis, e.g. retinopathies, and those associated with aberrant proliferation, e.g. cancer.

The terms “Wnt antagonist”, “Wnt inhibitor”, and “inhibitor of Wnt signaling” are used interchangeably herein to mean an agent that antagonizes, inhibits, suppresses, or negatively regulates Wnt modulation of a cell's activity. Likewise, the phrases “antagonizing Wnt signaling” and “inhibiting Wnt signaling” are used interchangeably herein to mean antagonizing, inhibiting, or otherwise negatively regulating Wnt modulation of a cell's activity. Wnt inhibitors may act anywhere along a Wnt signaling pathway to antagonize Wnt signaling. For example, Wnt inhibitors may antagonize activation of the Wnt co-receptors, for example by blocking Wnt binding; or they may antagonize the activity of a protein in a Wnt-responsive intracellular signaling cascade, e.g., the Wnt/β-catenin, Wnt/PCP or Wnt/Ca²⁺ signaling pathways, for example by promoting β-catenin degradation or preventing translocation of stabilized β-catenin to the nucleus.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent maybe administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired, particularly humans.

In methods of the present invention, Wnt signaling in a cell is antagonized by contacting the cell with an effective amount of a Wnt inhibitor. As defined above, Wnt inhibitors are agents that antagonize, inhibit, or negatively regulate Wnt modulation of a cell's activity. Agents suitable for inhibiting Wnt signaling in the present invention include small molecule compounds. Naturally occurring or synthetic small molecule compounds of interest include numerous chemical classes, such as organic molecules, e.g., small organic compounds having a molecular weight of more than 50 and less than about 2,500 daltons (Da). Candidate agents comprise functional groups for structural interaction with proteins, particularly hydrogen bonding, and typically include at least an amine, carbonyl, hydroxyl or carboxyl group, preferably at least two of the functional chemical groups. The candidate agents may include cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Exemplary of pharmaceutical agents suitable for this invention are those described in, “The Pharmacological Basis of Therapeutics,” Goodman and Gilman, McGraw-Hill, New York, N.Y., (1996), Ninth edition. Also included are toxins, and biological and chemical warfare agents, for example see Somani, S. M. (Ed.), “Chemical Warfare Agents,” Academic Press, New York, 1992). Small molecule compounds can be provided directly to the medium in which the cells are being cultured, for example as a solution in DMSO or other solvent.

Agents suitable for inhibiting Wnt signaling in the present invention also include nucleic acids, for example, nucleic acids that encode siRNA, shRNA or antisense molecules, or nucleic acids that encode polypeptides. Many vectors useful for transferring nucleic acids into target cells are available. The vectors may be maintained episomally, e.g. as plasmids, minicircle DNAs, virus-derived vectors such cytomegalovirus, adenovirus, etc., or they may be integrated into the target cell genome, through homologous recombination or random integration, e.g. retrovirus derived vectors such as MMLV, HIV-1, ALV, etc.

Vectors may be provided directly to the subject cells. In other words, the pluripotent cells are contacted with vectors comprising the nucleic acid of interest such that the vectors are taken up by the cells. Methods for contacting cells with nucleic acid vectors, such as electroporation, calcium chloride transfection, and lipofection, are well known in the art.

Alternatively, the nucleic acid of interest may be provided to the subject cells via a virus. In other words, the pluripotent cells are contacted with viral particles comprising the nucleic acid of interest. Retroviruses, for example, lentiviruses, are particularly suitable to the method of the invention. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein to be incorporated into the capsid, this envelope protein determining the specificity of the viral particle for the cells. Envelope proteins are of at least three types, ecotropic, amphotropic and xenotropic. Retroviruses packaged with ecotropic envelope protein, e.g. MMLV, are capable of infecting most murine and rat cell types, and are generated by using ecotropic packaging cell lines such as BOSC23 (Pear et al. (1993) P.N.A.S. 90:8392-8396). Retroviruses bearing amphotropic envelope protein, e.g. 4070A (Danos et al, supra.), are capable of infecting most mammalian cell types, including human, dog and mouse, and are generated by using amphotropic packaging cell lines such as PA12 (Miller et al. (1985) Mol. Cell. Biol. 5:431-437); PA317 (Miller et al. (1986) Mol. Cell. Biol. 6:2895-2902); GRIP (Danos et al. (1988) PNAS 85:6460-6464). Retroviruses packaged with xenotropic envelope protein, e.g. AKR env, are capable of infecting most mammalian cell types, except murine cells. The appropriate packaging cell line may be used to ensure that the subject CD33+ differentiated somatic cells are targeted by the packaged viral particles. Methods of introducing the retroviral vectors comprising the nucleic acid encoding the reprogramming factors into packaging cell lines and of collecting the viral particles that are generated by the packaging lines are well known in the art.

Vectors used for providing nucleic acid of interest to the subject cells will typically comprise suitable promoters for driving the expression, that is, transcriptional activation, of the nucleic acid of interest. This may include ubiquitously acting promoters, for example, the CMV-b-actin promoter, or inducible promoters, such as promoters that are active in particular cell populations or that respond to the presence of drugs such as tetracycline. By transcriptional activation, it is intended that transcription will be increased above basal levels in the target cell by at least about 10 fold, by at least about 100 fold, more usually by at least about 1000 fold. In addition, vectors used for providing reprogramming factors to the subject cells may include genes that must later be removed, e.g. using a recombinase system such as Cre/Lox, or the cells that express them are destroyed, e.g. by including genes that allow selective toxicity such as herpesvirus TK, bcl-xs, etc

Agents suitable for inhibiting Wnt signaling in the present invention also include polypeptides. Such polypeptides may optionally be fused to a polypeptide domain that increases solubility of the product. The domain may be linked to the polypeptide through a defined protease cleavage site, e.g. a TEV sequence, which is cleaved by TEV protease. The linker may also include one or more flexible sequences, e.g. from 1 to 10 glycine residues. In some embodiments, the cleavage of the fusion protein is performed in a buffer that maintains solubility of the product, e.g. in the presence of from 0.5 to 2 M urea, in the presence of polypeptides and/or polynucleotides that increase solubility, and the like. Domains of interest include endosomolytic domains, e.g. influenza HA domain; and other polypeptides that aid in production, e.g. IF2 domain, GST domain, GRPE domain, and the like.

If the polypeptide agent is to inhibit Wnt signaling intracellularly, the polypeptide may comprise the polypeptide sequences of interest fused to a polypeptide permeant domain. A number of permeant domains are known in the art and may be used in the non-integrating polypeptides of the present invention, including peptides, peptidomimetics, and non-peptide carriers. For example, a permeant peptide may be derived from the third alpha helix of Drosophila melanogaster transcription factor Antennapaedia, referred to as penetratin. As another example, the permeant peptide comprises the HIV-1 tat basic region amino acid sequence, which may include, for example, amino acids 49-57 of naturally-occurring tat protein. Other permeant domains include poly-arginine motifs, for example, the region of amino acids 34-56 of HIV-1 rev protein, nona-arginine, octa-arginine, and the like. (See, for example, Futaki et al. (2003) Curr Protein Pept Sci. 2003 April; 4(2): 87-96; and Wender et al. (2000) Proc. Natl. Acad. Sci. U.S.A 2000 Nov. 21; 97(24):13003-8; published U.S. Patent applications 20030220334; 20030083256; 20030032593; and 20030022831, herein specifically incorporated by reference for the teachings of translocation peptides and peptoids). The nona-arginine (R9) sequence is one of the more efficient PTDs that have been characterized (Wender et al. 2000; Uemura et al. 2002).

If the polypeptide agent is to inhibit Wnt signaling extracellularly, the polypeptide may be formulated for improved stability. For example, the peptides may be PEGylated, where the polyethyleneoxy group provides for enhanced lifetime in the blood stream. The SHBG polypeptide may be fused to another polypeptide to provide for added functionality, e.g. to increase the in vivo stability. Generally such fusion partners are a stable plasma protein, which may, for example, extend the in vivo plasma half-life of the SHBG polypeptide when present as a fusion, in particular wherein such a stable plasma protein is an immunoglobulin constant domain. In most cases where the stable plasma protein is normally found in a multimeric form, e.g., immunoglobulins or lipoproteins, in which the same or different polypeptide chains are normally disulfide and/or noncovalently bound to form an assembled multichain polypeptide, the fusions herein containing the SHBG polypeptide also will be produced and employed as a multimer having substantially the same structure as the stable plasma protein precursor. These multimers will be homogeneous with respect to the polypeptide agent they comprise, or they may contain more than one polypeptide agent.

Stable plasma proteins are proteins which typically exhibit in their native environment an extended half-life in the circulation, i.e. greater than about 20 hours. Examples of suitable stable plasma proteins are immunoglobulins, albumin, lipoproteins, apolipoproteins and transferrin. The polypeptide agent typically is fused to the plasma protein, e.g. IgG at the N-terminus of the plasma protein or fragment thereof which is capable of conferring an extended half-life upon the SHBG polypeptide. Increases of greater than about 100% on the plasma half-life of the SHBG polypeptide are satisfactory. Ordinarily, the SHBG polypeptide is fused C-terminally to the N-terminus of the constant region of immunoglobulins in place of the variable region(s) thereof, however N-terminal fusions may also find use. Typically, such fusions retain at least functionally active hinge, CH2 and CH3 domains of the constant region of an immunoglobulin heavy chain, which heavy chains may include IgG1, IgG2a, IgG2b, IgG3, IgG4, IgA, IgM, IgE, and IgD, usually one or a combination of proteins in the IgG class. Fusions are also made to the C-terminus of the Fc portion of a constant domain, or immediately N-terminal to the CH1 of the heavy chain or the corresponding region of the light chain. This ordinarily is accomplished by constructing the appropriate DNA sequence and expressing it in recombinant cell culture. Alternatively, the polypeptides may be synthesized according to known methods.

The site at which the fusion is made may be selected in order to optimize the biological activity, secretion or binding characteristics of the SHBG polypeptide. The optimal site will be determined by routine experimentation.

In some embodiments the hybrid immunoglobulins are assembled as monomers, or hetero- or homo-multimers, and particularly as dimers or tetramers. Generally, these assembled immunoglobulins will have known unit structures. A basic four chain structural unit is the form in which IgG, IgD, and IgE exist. A four chain unit is repeated in the higher molecular weight immunoglobulins; IgM generally exists as a pentamer of basic four-chain units held together by disulfide bonds. IgA immunoglobulin, and occasionally IgG immunoglobulin, may also exist in a multimeric form in serum. In the case of multimers, each four chain unit may be the same or different.

The polypeptide agent for use in the subject methods may be produced from eukaryotic produced by prokaryotic cells, it may be further processed by unfolding, e.g. heat denaturation, DTT reduction, etc. and may be further refolded, using methods known in the art.

Modifications of interest that do not alter primary sequence include chemical derivatization of polypeptides, e.g., acylation, acetylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Also included in the subject invention are polypeptides that have been modified using ordinary molecular biological techniques and synthetic chemistry so as to improve their resistance to proteolytic degradation or to optimize solubility properties or to render them more suitable as a therapeutic agent. Analogs of such polypeptides include those containing residues other than naturally occurring L-amino acids, e.g. D-amino acids or non-naturally occurring synthetic amino acids. D-amino acids may be substituted for some or all of the amino acid residues.

The subject polypeptides may be prepared by in vitro synthesis, using conventional methods as known in the art. Various commercial synthetic apparatuses are available, for example, automated synthesizers by Applied Biosystems, Inc., Beckman, etc. By using synthesizers, naturally-occurring amino acids may be substituted with unnatural amino acids. The particular sequence and the manner of preparation will be determined by convenience, economics, purity required, and the like.

If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

The polypeptide may be provided to the subject cells by standard protein transduction methods. In some cases, the protein transduction method includes contacting cells with a composition containing a carrier agent and the purified polypeptide. Examples of suitable carrier agents and methods for their use include, but are not limited to, commercially available reagents such as Chariot™ (Active Motif, Inc., Carlsbad, Calif.) described in U.S. Pat. No. 6,841,535; Bioport® (Gene Therapy Systems, Inc., San Diego, Calif.), GenomeONE (Cosmo Bio Co., Ltd., Tokyo, Japan), and ProteoJuice™ (Novagen, Madison, Wis.), or nanoparticle protein transduction reagents as described in, e.g., U.S. patent application Ser. No. 10/138,593.

Polypeptide agents may be provided to the subject cells individually or in combination. If provided in combination, they may be provided simultaneously either individually or as a single composition, that is, as a premixed composition, of polypeptide agents; alternatively, the polypeptide agents may be added to the subject cells sequentially at different times, at an effective dose. Dose optimization is readily performed by one of skill in the art.

Another example of polypeptide agents suitable for inhibiting Wnt signaling are antibodies. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. The specific or selective fit of a given structure and its specific epitope is sometimes referred to as a “lock and key” fit. The archetypal antibody molecule is the immunoglobulin, and all types of immunoglobulins, IgG, IgM, IgA, IgE, IgD, etc., from all sources, e.g. human, rodent, rabbit, cow, sheep, pig, dog, other mammal, chicken, other avians, etc., are considered to be “antibodies.” Antibodies utilized in the present invention may be either polyclonal antibodies or monoclonal antibodies. Antibodies are typically provided in the media in which the cells are cultured.

Agents may be obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

As discussed above, Wnt inhibitors may act anywhere along a Wnt signaling pathway to antagonize Wnt signaling. Of particular interest in some embodiments of the subject methods are Wnt inhibitors that negatively regulate Wnt receptor activation by acting extracellularly. Such antagonists may act by competitively inhibiting receptor activation, e.g. by binding to and sequestering Wnt ligands and/or by binding to and sequestering Frizzled receptors so as to prevent Wnt-Frizzled interaction. Alternatively, such antagonists may act by non-competitively inhibiting receptor activation, e.g. by binding to a domain on the Wnt receptor complex other than the Wnt interaction domain and modulating receptor activity.

One example of extracellular Wnt inhibitors is the soluble Frizzled Related Proteins (sFRPs). sFRPs are secreted proteins that share sequence similarity with Frizzled Receptors at their cysteine rich domain (CRD), but lack the transmembrane and intracellular domains. Through its CRD, sFRPs exhibit the ability to bind Wnt and heterodimerize with Frizzled, thereby potentially antagonizing Wnt signaling both by sequestering Wnts and by forming non-functional complexes with the Frizzled Receptor (Leyns, L, et al. (1997) Cell 88, 747-756; Wang, S, et al. (1997) Cell 88, 757-766; Rattner, A, et al. (1997) Proc Natl Acad Sci USA 94, 2859-2863; Ling K, et al. (1997) Proc Natl Acad Sci USA 94, 11196-11200; Bafico, A, et al. (1999) J Biol Chem 274, 16180-16187). Examples of FRP-related Wnt inhibitors may be found in U.S. Application No. 20080299136, incorporated herein by reference.

Another example of extracellular Wnt inhibitors is Dickkopf (Dkk). (Brott, B. K. et al. (2002) Mol. Cell Biol. 22: 6100-6110; Fedi, P. et al. (1999) J. Biol. Chem. 274: 19465-19472). The three members of the Dkk family (e.g., Dkk-1, Dkk-2 and Dkk-4) can antagonize Wnt signaling through inactivation of the cell surface receptor LRP-5 and LRP-6, essential components of the canonical pathway. (Mao, J. H. et al. (2001) Mol. Cell 7: 801-809; Pinson, K. I. et al. (2000) Nature 407: 535-538). Dkk forms a ternary complex with LRP5/6 and the single pass transmembrane receptors Kremen 1 (Krm-1) or Kremen 2 (Krm-2) (Mao et al. (2003) Gene 302: 179-183; Mao et al. (2002) Nature 417: 664-667; Mao et al. (2001) Nature 411: 321-325). This complex in turn undergoes endocytosis, thereby removing LRP5/6 receptors from the cell surface. As a result, Dkks can selectively antagonize canonical Wnt signaling, while not affecting non-canonical signaling. Examples of DKK-related Wnt inhibitors may be found in U.S. Application No. 20030068312, incorporated herein by reference.

A third example of extracellular Wnt inhibitors is Adenomatosis Polyposis Coli Down-regulated (Apcdd). Apcdd proteins are cell surface proteins that bind to Wnts, LRP5, and LRP6 by their extracellular domain and inhibit receptor activation. The terms “Apcdd gene product”, “Apcdd polypeptide”, “Apcdd peptide”, and “Apcdd protein” are used interchangeably herein to refer to native sequence Apcdd polypeptides, Apcdd polypeptide variants, Apcdd polypeptide fragments and chimeric Apcdd polypeptides. Native sequence Apcdd polypeptides include the protein Apcdd1 (also referred to as Drapc1), the sequence for which may be found at Genbank Accession No. NM_{—}153000 (SEQ ID NO:1, SEQ ID NO:2), and which is expressed in a broad range of cell types; see, for example, Jukkola, T., et al. (2004) Gene Expr. Patterns 4, 755-762, the disclosure of which is incorporated herein by reference. Native sequence Apcdd polypeptides also include the protein Apcdd1-like (Apcdd1L), the sequence for which may be found at Genbank Accession No. NM_{—}153360 (SEQ ID NO:3, SEQ ID NO:4). Apcdd polypeptides comprise an N-terminal signal peptide, a large extracellular domain with an N-glycosylation site, a C-terminal transmembrane domain, and a short cytoplasmic tail.

In some embodiments, the Wnt inhibitor is an Apcdd peptide. An Apcdd peptide is a peptide comprising Apcdd sequence that inhibits Wnt activity. An Apcdd peptide may comprise a polypeptide having a sequence identity of 50%, 55%, 60%, 65%, 70%, 75%, 80%, 85%, 90%, 95%, 98% or 100% to the full polypeptide sequence of Apcdd1 or Apcdd1L. An Apcdd peptide may comprise a polypeptide having a sequence identity of 50%, 55%, 60%, 65%, 70%, 75%, 80%, 85%, 90%, 95%, 98% or 100% to the extracellular domain of Apcdd1 or Apcdd1L (i.e., the full-length polypeptide minus the transmembrane domain and intracellular domain). An Apcdd peptide may comprise a polypeptide having a sequence identity of 50%, 55%, 60%, 65%, 70%, 75%, 80%, 85%, 90%, 95%, 98% or 100% to the Wnt- and/or LRP-binding domain of Apcdd1 or Apcdd1L. Such fragments are readily identifiable to one of ordinary skill in the art using common biochemical and genetic techniques that are well known in the art. Also encompassed by the subject invention are nucleic acids encoding polypeptides having a sequence identity of 50%, 55%, 60%, 65%, 70%, 75%, 80%, 85%, 90%, 95%, 98% or 100% to the polypeptide sequence of full length Apcdd1 or Apcdd1L, its extracellular domain, or its Wnt- and/or LRP-binding domains, and vectors comprising these nucleic acids. In some embodiments, a Wnt antagonist is a chimeric polypeptide comprising the Apcdd polypeptide component and an immunoglobulin Fc domain. In certain embodiments, the Fc domain is a human IgG1, IgG2, IgG3 or IgG4 Fc domain. In one embodiment, the Fc domain is a human IgG1 Fc domain.

In methods of the present invention, cells are contacted with an effective amount of the Wnt inhibitor so as to inhibit Wnt signaling. Biochemically speaking, an effective amount or effective dose of a Wnt inhibitor is an amount of inhibitor to decrease or attenuate Wnt signaling in a cell by at least 30%, at least 40%, at least 50%, at least 60%, at least 70%, at least 80%, at least 90%, at least 95%, or by 100%. In other words, the responsiveness to Wnt signaling of a cell that has been contacted with an effective amount or effective dose of a Wnt antagonist will be about 70% or less, about 60% or less, about 50% or less, about 40% or less, about 30% or less, about 20% or less, about 10% or less, about 5% or less, or will be about 0%, i.e. negligible, the strength of the responsiveness observed of a cell that has not been contacted with an effective amount/dose of a Wnt antagonist. The amount of modulation of a cell's activity by Wnt, that is, the responsiveness of a cell to Wnt signaling, can be determined by a number of ways known to one of ordinary skill in the art of Wnt biology. For example, the amount of phosphorylated β-catenin in a cell may be measured; the amount of cytosolic β-catenin in a cell may be measured; or the amount of activity of the transcription factors that are normally activated by Wnt signaling, e.g. TCF/LEF, may be measured, for example by measuring the RNA or protein levels of genes that are the transcriptional targets of TCF/LEF, or by transfecting/infecting the cell with a nucleic acid vector comprising a TCF binding site (TOP) operably linked to a reporter protein such as luciferase (TOPFlash), EGFP (TOP-EGFP), etc. and qualitatively or quantitatively measuring the amount of reporter protein that is produced. In this way, the antagonistic effect of the agent may be confirmed.

In a clinical sense, an effective dose of a Wnt inhibitor is the dose that, when administered for a suitable period of time, usually at least about one week, and maybe about two weeks, or more, up to a period of about 4 weeks, 8 weeks, or longer will evidence an alteration in the symptoms associated with undesired Wnt signaling. For example, an effective dose is the dose that when administered for a suitable period of time, usually at least about one week, and may be about two weeks, or more, up to a period of about 4 weeks, 8 weeks, or longer will slow or even halt neovascularization in the eye of a patient suffering from diabetic retinopathy or tumor growth in a patient suffering from cancer. In some embodiments, an effective dose may not only slow or halt the progression of the disease condition but may also induce the reversal of the condition. For example, an effective dose of a Wnt inhibitor will not only halt neovasculaturization in the eye of a patient suffering from diabetic retinopathy, but will reduce the amount of existing vasculature. Similarly, an effective dose of a Wnt inhibitor will not only halt tumor growth in a cancer patient, but will reduce the size of the tumor(s). It will be understood by those of skill in the art that an initial dose may be administered for such periods of time, followed by maintenance doses, which, in some cases, will be at a reduced dosage.

In methods of the present invention, an effective amount of Wnt inhibitor is provided to cells, e.g. by contacting the cell with an effective amount of Wnt inhibitor, by providing the cells with an effective amount of Wnt inhibitor, or by administering to the cell an effecting amount of Wnt inhibitor, so as to inhibit Wnt signaling in those cells.

Cells suitable for use in the method include any cell that is responsive to Wnt signaling. The responsiveness of a cell to a Wnt may be readily determined by one of ordinary skill in the art by methods known in the art and set forth herein. The cells may be in vitro, that is, in culture, or they may be in vivo, that is, in a subject. Cells may be from any organism, but are preferably from a mammal, including humans, domestic and farm animals, and zoo, laboratory or pet animals, such as dogs, cats, cattle, horses, sheep, pigs, goats, rabbits, rats, mice etc. Preferably, the mammal is human. Cells may be from any tissue. Of particular interest in some embodiments are cells that reside or resided in the Central Nervous System (CNS), that is, a CNS cell. The term “central nervous system” (CNS) as used herein refers any of the tissues that are derived from the neural tube, for example, the brain, the spinal cord, and the eye. CNS cells are any cells that reside in any of these tissues, for example neural stem cells, neural progenitor cells, neurons, glia, endothelial cells, pericytes, photoreceptors, retinal pigment epithelial cells, etc.

Cells of particular interest are those that are responsive to Wnt signaling and are associated with aberrant angiogenesis or aberrant cell proliferation, particularly as they may relate to Wnt-mediated disease conditions described below. As an example, cells of interest include endothelial cells, which are the cells that line the interior surface of blood vessels, and which, when aberrantly active, may be associated with aberrant angiogenesis. As another example, cells of interest include cancer cells, e.g. a cancer stem cell, which is a type of cancer cell that possesses characteristics associated with normal stem cells, namely the ability to give rise to all cell types found in a particular cancer sample, and which is associated with aberrant cell proliferation.

Cells in vitro may be contacted with Wnt inhibitor by any of a number of well-known methods in the art. For example, polypeptides (including antibodies) or small molecule agents may be provided to the cells in the media in which the cells are being cultured. Nucleic acids encoding the Wnt inhibitor may be provided to the cells on vectors under conditions that are well known in the art for promoting their uptake, for example electroporation, calcium chloride transfection, and lipofection. Alternatively, nucleic acids encoding the Wnt inhibitor may be provided to the cells via a virus, i.e. the cells are contacted with viral particles comprising nucleic acids encoding the Wnt inhibitor. Retroviruses, for example, lentiviruses, are particularly suitable to the method of the invention, as they can be used to transfect non-dividing cells (see, for example, Uchida et al. (1998) P.N.A.S. 95(20):11939-44). Commonly used retroviral vectors are “defective”, i.e. unable to produce viral proteins required for productive infection. Rather, replication of the vector requires growth in a packaging cell line.

Likewise, cells in vivo may be contacted with a Wnt inhibitor by any of a number of well-known methods in the art for the administration of peptides, small molecules and nucleic acids to a subject. The Wnt inhibitor can be incorporated into a variety of formulations. More particularly, the compounds of the present invention can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the Wnt inhibitor can be achieved in various ways, including oral, buccal, rectal, parenteral, intraperitoneal, intradermal, transdermal, intracheal, etc., administration. The active agent may be systemic after administration or may be localized by the use of regional administration, intramural administration, or use of an implant that acts to retain the active dose at the site of implantation. The active agent may be formulated for immediate activity or it may be formulated for sustained release.

For some conditions, particularly central nervous system conditions, it may be necessary to formulate agents to cross the blood-brain barrier (BBB). One strategy for drug delivery through the blood-brain barrier (BBB) entails disruption of the BBB, either by osmotic means such as mannitol or leukotrienes, or biochemically by the use of vasoactive substances such as bradykinin. The potential for using BBB opening to target specific agents to brain tumors is also an option. A BBB disrupting agent can be co-administered with the therapeutic compositions of the invention when the compositions are administered by intravascular injection. Other strategies to go through the BBB may entail the use of endogenous transport systems, including Caveolin-1 mediated transcytosis, carrier-mediated transporters such as glucose and amino acid carriers, receptor-mediated transcytosis for insulin or transferrin, and active efflux transporters such as p-glycoprotein. Active transport moieties may also be conjugated to the therapeutic compounds for use in the invention to facilitate transport across the endothelial wall of the blood vessel. Alternatively, drug delivery of therapeutics agents behind the BBB may be by local delivery, for example by intrathecal delivery, e.g. through an Ommaya reservoir (see e.g. U.S. Pat. Nos. 5,222,982 and 5,385,582, incorporated herein by reference); by bolus injection, e.g. by a syringe, e.g. intravitreally or intracranially; by continuous infusion, e.g. by cannulation, e.g. with convection (see e.g. US Application No. 20070254842, incorporated here by reference); or by implanting a device upon which the agent has been reversibly affixed (see e.g. US Application Nos. 20080081064 and 20090196903, incorporated herein by reference).

The calculation of the effective amount or effective dose of Wnt inhibitor to be administered is within the skill of one of ordinary skill in the art, and will be routine to those persons skilled in the art. Needless to say, the final amount to be administered will be dependent upon the route of administration and upon the nature of the disorder or condition that is to be treated.

For inclusion in a medicament, Wnt inhibitors may be obtained from a suitable commercial source. As a general proposition, the total pharmaceutically effective amount of the Wnt inhibitor compound administered parenterally per dose will be in a range that can be measured by a dose response curve.

A Wnt inhibitor to be used for therapeutic administration must be sterile. Sterility is readily accomplished by filtration through sterile filtration membranes (e.g., 0.2 □m membranes). Therapeutic compositions generally are placed into a container having a sterile access port, for example, an intravenous solution bag or vial having a stopper pierceable by a hypodermic injection needle. The Wnt inhibitor ordinarily will be stored in unit or multi-dose containers, for example, sealed ampules or vials, as an aqueous solution or as a lyophilized formulation for reconstitution. As an example of a lyophilized formulation, 10-mL vials are filled with 5 ml of sterile-filtered 1% (w/v) aqueous solution of compound, and the resulting mixture is lyophilized. The infusion solution is prepared by reconstituting the lyophilized compound using bacteriostatic Water-for-Injection.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with low toxicity. The dosage can vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The effective amount of a therapeutic composition to be given to a particular patient will depend on a variety of factors, several of which will differ from patient to patient. A competent clinician will be able to determine an effective amount of a therapeutic agent to administer to a patient to halt or reverse the progression the disease condition as required. Utilizing LD₅₀ animal data, and other information available for the agent, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions which are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

Mammalian species that may be treated with the present methods include canines and felines; equines; bovines; ovines; etc. and primates, particularly humans. Animal models, particularly small mammals, e.g. murine, lagomorpha, etc. may be used for experimental investigations. Other uses include investigations where it is desirable to investigate a specific effect in the absence of Wnt signaling.

The methods of the present invention also find use in combined therapies. For example, a number of agents may be useful in the treatment of aberrant angiogenesis, e.g. angiostatin, endostatin, VEGF inhibitors, etc. Likewise, a number of agents may be useful in the treatment of cancer, e.g. chemotherapeutic agents, kinase inhibitors, etc. The combined use of the Wnt inhibitors of the present invention and these other agents may have the advantages that the required dosages for the individual drugs is lower, and the effect of the different drugs complementary.

Conditions of interest. As alluded to above, the present invention finds use in the treatment of mammals, such as human patients, suffering from Wnt-mediated disease conditions such as disorders associated with aberrant angiogenesis in the CNS or aberrant cell proliferation. Patients suffering from diseases characterized by such conditions will benefit greatly by a treatment protocol of the pending claimed invention.

For example, methods and compositions of the present invention find use in inhibiting aberrant angiogenesis in a CNS cell. The term “angiogenesis” is used to describe the biological process by which new blood vessels grow or sprout from pre-existing vessels. Angiogenesis plays a critical role in the elaboration of vasculature both during embryogenesis and in the mature organism, for example, in wound healing. However, there are many disease states that are driven by persistent unregulated or improperly regulated angiogenesis. In such disease states, this aberrant angiogenesis may either cause a particular disease or exacerbate an existing pathological condition. For example, choroidal neovascularization (CNV) in the eye and the subsequent retinopathy has been implicated as the most common cause of blindness and underlies the pathology of a number of ocular diseases, most notably diabetic retinopathy and age related macular degeneration (AMD, ARMD), particularly wet/exudative age related macular degeneration. Wnt inhibitors find use in treating, i.e., arresting, the development or progression of, disease conditions of the CNS wherein aberrant angiogenesis is a contributing factor.

A Wnt antagonist that inhibits aberrant angiogenesis/neovascularization in the CNS is one which results in measurable inhibition of the development of new vasculature, for example tube formation by endothelial cells in culture or blood vessel formation in a subject. Preferred Wnt antagonists inhibit the rate of development of new vasculature by at least about 10%, at least about 20%, preferably from about 20% to about 50%, and even more preferably, by greater than 50% (e.g., from about 50% to about 100%) as compared to the appropriate control, the control typically being cells not treated with the Wnt antagonist molecule being tested. The Wnt antagonist is inhibitory in vivo if administration of the Wnt antagonist at about 1 ug/kg to about 100 mg/kg body weight results in a slowing or cessation of the development of neovasculature within about 5 days to 6 months from the first administration of the Wnt inhibitor, preferably within about 5 days to about 2 months. Neovasculature development, i.e. angiogenesis, may be observed by a number of ways that are well-known in the art and that will be obvious to the ordinary skilled artisan. For example, the inhibition of choroidal neovascularization may be readily observed directly by fundus photography, or indirectly by assaying for improved scoring on visual acuity tests.

Methods and compositions of the present invention also find use in inhibiting cell proliferative disorder associated with aberrant Wnt signaling activity, for example cancer, such as of gliomas, medulloblastomas, colon cancer, colorectal cancer, breast cancer, or leukemia. The term “cancer” refers to the physiological condition in mammals that is typically characterized by unregulated cell growth/proliferation. Examples of cancer include, but are not limited to: carcinoma, lymphoma, blastoma, and leukemia. More particular examples of cancers include, but are not limited to: chronic lymphocytic leukemia (CLL), lung, including non small cell (NSCLC), breast, ovarian, cervical, endometrial, prostate, colorectal, intestinal carcinoid, bladder, gastric, pancreatic, hepatic (hepatocellular), hepatoblastoma, esophageal, pulmonary adenocarcinoma, mesothelioma, synovial sarcoma, osteosarcoma, head and neck squamous cell carcinoma, juvenile nasopharyngeal angiofibromas, liposarcoma, thyroid, melanoma, basal cell carcinoma (BCC), medulloblastoma and desmoid.

A Wnt antagonist that inhibits the growth of a tumor is one which results in measurable reduction in the rate of proliferation of cancer cells in vitro or growth inhibition of a tumor in vivo. For example, preferred growth inhibitory Wnt antagonists will inhibit growth of tumor by at least about 5%, at least about 10%, at least about 20%, preferably from about 20% to about 50%, and even more preferably, by greater than 50% (e.g., from about 50% to about 100%) as compared to the appropriate control, the control typically being cancer cells not treated with the Wnt antagonist molecule being tested. The Wnt antagonist is growth inhibitory in vivo if administration of the Wnt antagonist at about 1 μg/kg to about 100 mg/kg body weight results in reduction in tumor size or cell proliferation within about 5 days to 3 months from the first administration of the antibody, preferably within about 5 to 30 days. In a specific aspect, the tumor size is reduced relative to its size at the start of therapy.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Hereditary hypotrichosis simplex (HHS) is a rare autosomal dominant form of hereditary hair loss characterized by hair follicle (HF) miniaturization. Using genetic linkage analysis, we mapped a novel locus for HHS to chromosome 18p11.22, and identified a mutation (L9R) in the APCDD1 gene in three families. We show that APCDD1 is a membrane-bound glycoprotein that is abundantly expressed in human HFs, and can interact in vitro with WNT3A and LRP5, two essential components of Wnt signaling. Functional studies revealed that APCDD1 inhibits Wnt signaling in a cell-autonomous mariner and functions upstream of β-catenin. Moreover, APCDD1 represses activation of Wnt reporters and target genes, and inhibits the biological effects of Wnt signaling during both the generation of neurons from progenitors in the developing chick nervous system, and axis specification in Xenopus embryos. The mutation L9R is located in the signal peptide of APCDD1, and perturbs its translational processing from ER to the plasma membrane. L9R-APCDD1 functions in a dominant-negative manner to inhibit the stability and membrane localization of the wild-type protein. These findings describe a novel inhibitor of the Wnt signaling pathway.

### Methods

Clinical details and DNA extraction. Informed consent was obtained from all subjects and approval for this study was provided by the Institutional Review Board of Columbia University. The study was conducted in adherence to the Declaration of Helsinki Principles. Peripheral blood samples were collected from the family members as well as unrelated healthy control individuals of Pakistani and European origin (200 individuals each). Genomic DNA was isolated from these samples using the PUREGENE DNA isolation kit (Gentra System).

Linkage analysis. Genome-wide genotyping was performed with the Affymetrix Human Mapping 10K 2.0 Array. Quality control and data analysis was performed with Genespring GT (Agilent software). Briefly, SNPs that violated Mendelian inheritance pattern were removed from the data set prior to analysis. Haplotypes were inferred from raw genotype data. By analyzing haplotypes rather than individual SNPs, Type I error introduced by linkage disequilibrium between markers is mitigated. Finally, haplotypes were analyzed for linkage under the assumption of a fully penetrant disease gene with a frequency of 0.001 transmitted by a dominant mode of inheritance.

Mutation analysis of the APCDD1 gene. Exons and exon-intron boundaries of APCDD1 gene were amplified by PCR with gene-specific primers listed in Table 1, below. Exon 1 and the adjacent boundary sequences of the APCDD1 gene were amplified using Platinum® Taq DNA Polymerase High Fidelity (Invitrogen). Due to the high G/C content, DMSO (final 5%) and MgSO₄ (final 1.6 mM) were added to the PCR reaction. The amplification conditions were 94° C. for 2 min, followed by 35 cycles of 94° C. for 30 sec, 61° C. for 30 sec, and 68° C. for 50 sec, with a final extension at 68° C. for 7 min. Other exons, as well as the exon-intron boundaries of the APCDD1 gene, were amplified using Platinum® PCR SuperMix (Invitrogen). The amplification conditions were 94° C. for 2 min, followed by 35 cycles of 94° C. for 30 sec, 56° C. for 30 sec, and 72° C. for 50 sec, with a final extension at 72° C. for 7 min. The PCR products were directly sequenced in an ABI Prism 310 Automated Sequencer, using the ABI Prism Big Dye Terminator Cycle Sequencing Ready Reaction Kit (PE Applied Biosystems).

In order to screen for the mutation 26T>G (L9R), a part of exon 1 and intron 1 of the APCDD1 gene was amplified by PCR using Platinum® Taq DNA Polymerase High Fidelity (Invitrogen) and the following primers: forward (5′-CCAGAGCAGGACTGGAAATG-3′) (SEQ ID NO:5), reverse (5′-CGCCAAGGGGACAGTGTAG-3′) (SEQ ID NO:6). DMSO (final 5%) and MgSO₄ (final 1.6 mM) were added to the PCR reaction. The amplification conditions were 94° C. for 2 min, followed by 35 cycles of 94° C. for 30 sec, 59° C. for 30 sec, and 68° C. for 30 sec, with a final extension at 68° C. for 7 min. The amplified PCR products, 191 bp in size, were digested with DdeI at 37° C. overnight, and run on 2.0% agarose gels.

TABLE 1
primers used in this study. “AT”, Annealing Temp.
in ° C.; “Prod Size”, Product size in basepairs.

| | | Forward | SEQ | Reverse | SEQ | | Prod |
|---|---|---|---|---|---|---|---|
| | Position | primer | ID | primer | ID | AT | Size |
| Primer | (bp) | (5′-3″) | NO | (5′-3′) | NO | ° C. | (bp) |

Primers to amplify additional microsatellite markers

| | | | | | | | |
|---|---|---|---|---|---|---|---|
| RAB31- | 9,847,609- | GCAAATGAATAC | 39 | CACCAGGCCTAG | 40 | 55 | 148 |
| MS | 9,847,756 | ACTAACTAGCC | | GCTAAAATG | | | |
| | | A | | | | | |
| APCDD1- | 10,454,280- | CCAGTGAGACCT | 41 | TTGCTATACTTTA | 42 | 55 | 124 |
| MS | 10,454,403 | TAAAAGCTTCA | | GGAGCCAGAA | | | |
| GNAL-MS | 11,693,739- | GGCTTGGGTACA | 43 | CAGAGCTTCCTGC | 44 | 55 | 116 |
| | 11,693,854 | ATAATGAGCT | | ACTTCAC | | | |

Primers to amplify candidate genes form genomic DNA

| | | | | | | |
|---|---|---|---|---|---|---|
| APCDD1 ex1 | CGACGCGCCCTT | 45 | CGCCAAGGGGAC | 46 | 61 | 668 |
| | TCAAGTCT | | AGTGTAG | | | |
| APCDD1 ex2 | CACTGTCTGCTG | 47 | GGTCTTCCATAGT | 48 | 56 | 328 |
| | CAGAGGT | | GGTGTGC | | | |
| APCDD1 ex3 | GAATCTCTTTCCC | 49 | TGGCAAGCCTTTA | 50 | 56 | 659 |
| | ATACCTTCAG | | AGAAGGATC | | | |
| APCDD1 ex4 | CGGAAGCATGTG | 51 | AAGAGGTCCTTTC | 52 | 56 | 481 |
| | TGCACTG | | CCTCAGC | | | |
| APCDD1 ex5 | GTCTAGTTAGAG | 53 | TTCTCTGGCATTC | 54 | 56 | 651 |
| | TGTGGCCAG | | AAGTGCATC | | | |
| RAB31 ex6 | CAAAGCAGGAGT | 55 | TGTGCTCACTCTT | 56 | 55 | 302 |
| | GTTGAAGCA | | CTCAAAGTG | | | |
| TXNDC2 ex1 | GAGGGAAAACCA | 57 | CTACAACTTTCCC | 58 | 55 | 323 |
| | ACTGTAACGT | | TTCTTGGTTC | | | |
| TXNDC2 ex2 | CAGACTTCATTC | 59 | TACAAGCAGTGCC | 60 | 59 | 1,802 |
| | GTGATCTCGA | | ATTTGGACAT | | | |
| VAPA ex1 | AGCCTGGCCTCG | 61 | AAAGCAGCCGGG | 62 | 55 | 335 |
| | TCCTAGA | | AAAGGGAA | | | |
| VAPA ex2 | GAAAGGCAGTGT | 63 | TATTTCCCTCCCT | 64 | 55 | 339 |
| | TAGTAGCCAT | | CCAGATG | | | |
| VAPA ex3 | GTTTAAGGCAAA | 65 | ACTGCTAAACAAC | 66 | 55 | 275 |
| | TCCCAGACTT | | ACCCACAGT | | | |
| VAPA ex4 | GTCCCGTGAGGT | 67 | GGAACTGAGAGCT | 68 | 55 | 203 |
| | GAAACTTA | | CAACAGC | | | |
| VAPA ex5 | CAGTCATTCCCA | 69 | CACAATACCACTT | 70 | 55 | 299 |
| | ATATCATGCAG | | ATCTGCTAGG | | | |
| VAPA ex6 | ATCTGACTGCTG | 71 | TGGCCTAATAACA | 72 | 55 | 383 |
| | ACATGTACTG | | CTCACATGTC | | | |
| VAPA ex7 | CACTAATCTACTT | 73 | CGTGGGCCATACT | 74 | 55 | 350 |
| | TCCGTCCCTA | | ACCAATG | | | |
| NAPG ex1 | TGTCGCGCTGCA | 75 | GGAGTGTGACCC | 76 | 55 | 337 |
| | CCAGCTT | | AGAGGAC | | | |
| NAPG ex2 | GACCTAGAAGGT | 77 | GCCTTATAGAAAG | 78 | 55 | 249 |
| | CATTACAAGC | | CATATGAGTAA | | | |
| | | | GT | | | |
| NAPG ex3 | CTGATTTGTTGC | 79 | GCGAACCAGTGT | 80 | 55 | 527 |
| | CAGTAGTCAA | | GGACCTTA | | | |
| | C | | | | | |
| NAPG ex4 | TGGAACCAACTG | 81 | TTACAGTTAGGAG | 82 | 55 | 306 |
| | GGTCAGTG | | TAAGTCTTGGT | | | |
| NAPG ex5 | TATGTTGTGCAT | 83 | CTTGCCTTACAGT | 84 | 55 | 261 |
| | GAGCCCATTG | | AAGGAAGCT | | | |
| NAPG ex6-8 | CTGCACATGTGT | 85 | TGGTGTGTCTACA | 86 | 55 | 869 |
| | CCCAGAAC | | GCTTATACC | | | |
| NAPG ex9 | AAGTACATTTCAC | 87 | AAGCTGTAGTGGG | 88 | 55 | 318 |
| | AGGGACCT | | CTTACTCTA | | | |
| NAPG ex10-11 | TCCCGTGGAAGT | 89 | GGTGAAATTCAAT | 90 | 55 | 1,109 |
| | TACTATAGCA | | GCAAGTGGTC | | | |
| NAPG ex12 | CCACCATGGTCA | 91 | GGATATCCCTAAT | 92 | 55 | 405 |
| | TGAGGCAT | | CTATTCCCAAG | | | |
| FAM38B ex1 | GCTCCACCACCC | 93 | CTGCCTTACGCAG | 94 | 55 | 299 |
| | ATCTTATG | | ATAACATTTG | | | |
| FAM38B ex2-3 | GAAGGTGGGACC | 95 | CGTTGACTATTGA | 96 | 58 | 753 |
| | TGACTGGA | | CCCAAACCT | | | |
| FAM38B ex4 | GTTGGAATGATG | 97 | GTTCCACAACGAT | 98 | 55 | 395 |
| | AAGGGGAAAT | | TCCCACTG | | | |
| | TC | | | | | |
| FAM38B ex5 | CAGTTAAGGAAC | 99 | TGCCTTCATTGTG | 100 | 55 | 331 |
| | TGCTTGAGCT | | AGCAGAAGT | | | |
| FAM38B ex6 | CTCAGGATGCAG | 101 | TTCCCACCACCTC | 102 | 55 | 250 |
| | AGAAGAGC | | AGGCCAT | | | |
| FAM38B ex7 | AGTGCAATGGAA | 103 | CCCAACTCCATGT | 104 | 55 | 389 |
| | CACTCAAGAC | | TTAGACTGG | | | |
| FAM38B ex8 | TGACCGTTGTGG | 105 | GAAGTTGCATTCC | 106 | 55 | 351 |
| | ACCCAATG | | TCATACACATG | | | |
| FAM38B ex9 | GAGAGTTTTGAC | 107 | TGTCACACAGGGA | 108 | 55 | 371 |
| | TGTAATAGGAA | | GAGATAACT | | | |
| | C | | | | | |
| FAM38B ex10 | TTAGTAACCAGAT | 109 | CCCACTCTCAACT | 110 | 55 | 345 |
| | TTTGCCATCTG | | TTACATGATAC | | | |
| FAM38B ex11 | CTTCTACAGAATT | 111 | TCCGTCGAACTAG | 112 | 55 | 455 |
| | TGAGGGAAAG | | AAATGCTTAG | | | |
| | T | | | | | |
| AMAC1L1 | CCACCTCAGGAT | 113 | CCTCTGAATGGTT | 114 | 55 | 1239 |
| | AGTTCCAG | | AGGTCCTTA | | | |
| GNAL ex1 | CTGGGCGTTAGC | 115 | CCCACAGTTTAAAA | 116 | 55 | 843 |
| | AAGTGATC | | CGCTCTGTA | | | |

Primers used in RT-PCR experiments

| | | | | | | |
|---|---|---|---|---|---|---|
| APCDD1-rtpcr | ATGCCACCCAGA | 117 | GATGGTCAGGTCT | 118 | 56 | 155 |
| | GGATGTTC | | GCCTTTG | | | |
| KRT15-rtpcr | GGGTTTTGGTGG | 119 | TCGTGGTTCTTCT | 120 | 56 | 74 |
| | TGGCTTTG | | TCAGGTAGGC | | | |
| B2M-rtpcr | CACAGCCCAAGA | 121 | GCATAAAGTGTAA | 122 | 56 | 143 |
| | TAGTTAAGTG | | GTGTATAAGCA | | | |
| | | | TA | | | |
| APCDD1L-rtpcr | GTGGAGGAGCTG | 123 | GGCAATGAGGCC | 124 | 56 | 135 |
| | TACCTGG | | ACAGGCT | | | |
| LRP5-rtpcr | GACCCAGCCCTT | 125 | TGTGGACGTTGAT | 126 | 56 | 138 |
| | TGTTTTGAC | | GGTATTGGT | | | |
| WNT3A-rtpcr | GCCCCACTCGGA | 127 | GAGGAATACTGTG | 128 | 56 | 98 |
| | TACTTCTTACT | | GCCCAACA | | | |

Primers used to generate the probes for in situ hybridization

| | | | | | | |
|---|---|---|---|---|---|---|
| APCDD1-ISH | CCAGAGCAGGAC | 129 | ACGTTGTGCAGCT | 130 | 58 | 466 |
| | TGGAAATG | | GGTAGTC | | | |

Cell culture. HEK293T (human embryonic kidney) cells were cultured in Dulbecco's modified Eagle's medium (DMEM; GIBCO) supplemented with 10% fetal bovine serum (FBS; GIBCO), 100 IU/ml penicillin, and 100 μg/ml streptomycin. For transfection experiments, dishes were coated with a coating medium containing 0.01 mg/ml of fibronectin (Sigma) and 0.03 mg/ml of type I collagen (Sigma) before seeding the cells in order to prevent detachment of the cells.

Anti-APCDD1 antibodies. A mouse polyclonal anti-human APCDD1 (APCDD1) antibody was purchased from Abnova Corporation. This antibody was raised against the full-length human APCDD1 protein. We performed epitope-mapping using three truncated GST-APCDD1 proteins (amino acid residues 1-171, 166-336, and 331-514), and confirmed that the epitope of the antibody exists between amino acid residues 166 and 336 of the human APCDD1, which corresponds to the middle portion of the extracellular domain. An affinity-purified rabbit polyclonal anti-mouse APCDD1 antibody was produced by immunizing the synthetic peptide, CQRPSDGSSPDRPEKRATSY (SEQ ID NO:7) (corresponding to the C-terminus of the extracellular domain of the mouse APCDD1 protein, aa residues 441-459 conjugated to KLH) (Pierce, Rockford, Ill.). This region is completely conserved among mouse and human APCDD1 proteins. The antibody was affinity-purified from the serum using the Sulfolink immobilization column (Pierce). This antibody strongly recognizes human APCDD1 protein in western blots and immunofluorescence.

RT-PCR in human scalp skin and plucked hairs. Total RNA were isolated from scalp skin and plucked scalp hairs of healthy control individuals using the RNeasy® Minikit (Quiagen). 2 (g of total RNA was reverse-transcribed with oligo-dT primers and SuperScript™ III (Invitrogen). The cDNAs were amplified by PCR using Platinum® PCR SuperMix and primer pairs for APCDD1, APCDD1L, keratin 15 (KRT15), LRP5, WNT3A, and β-2 microglobulin (B2M) genes (Table 1). Primers for the KRT15, LRP5, and WNT3A genes were designed as described previously (Pasternack, S. M., et al. (2008) Nat. Genet. 40, 329-334; Konigshoff, M., et al. (2008) PLoS ONE 3, e2142). The amplification conditions were 94° C. for 2 min, followed by 35 cycles of 94° C. for 30 sec, 58° C. for 30 sec and 72° C. for 30 sec, with a final extension at 72° C. for 7 min. PCR products were run on 1.5% agarose gels.

Expression vectors. To generate the expression construct for the human APCDD1 (pCXN2.1-WT-APCDD1), the full length APCDD1 cDNA sequences were amplified by PCR using the first strand cDNA from human scalp skin as a template and the following primers: APCDD1-F-XhoI (5′-AAAACTCGAGCCAGAGCAGGACTGGAAATG-3′, SEQ ID NO:8) and APCDD1-R-NheI (5′-AAAAGCTAGCCTATCTGCGGATGTTCCAATGC-3′; SEQ ID NO: 9). To generate the constructs for the C-terminal hemagglutin (HA)-tagged (pCXN2.1-WT-APCDD1HA) and the C-terminal Flag-tagged (pCXN2.1-WT-APCDD1-Flag) APCDD1, the following reverse primers were used: APCDD1-R-HA-NheI (5′-AAAAGCTAGCTCAGGCGTAGTCGGGCACGTCGTAGGGGTATCTGCGGATGITCCAATGC-3′; SEQ ID NO:10) and APCDD1-R-Flag-NheI (5′-AAAAGCTAGCTCACTTATCGTCGTCATCCITGTAATCTCTGCGGATGITCCAATGC-3′; SEQ ID NO:11), respectively. L9R and L9V mutant APCDD1 sequences were PCR amplified using the following forward primers: APCDD1-L9R-F-XhoI (5′-AAAACTCGAGCCAGAGCAGGACTGGAAATGTCCTGGCCGCGCCGCCTCCTGCGCAGAT-3′; SEQ ID NO:12) and APCDD1-L9V-F-XhoI (5′-AAAACTCGAGCCAGAGCAGGACTGGAAATGTCCTGGCCGCGCCGCCTCCTGGTCAGAT-3′; SEQ ID NO:13), respectively. Note that T>G and C>G substitutions were introduced into the primers, respectively (shown in bold and underlined). For generating the expression constructs for truncated APCDD1 (aa residues 1-486) with the C-terminal HA-tag (pCXN2.1-APCDD1-ΔTM-HA) and the C-terminal Flag-tag (pCXN2.1-APCDD1-ΔTM-Flag), the following reverse primers were used: APCDD1-ΔTM-R-HA-NheI (5′-AAAAGCTAGCTCAGGCGTAGTCGGGCACGTCGTAGGGGTAGCCATACAGGCTGCTTCCACT-3′; SEQ ID NO:14) and APCDD1-ΔTM-R-Flag-NheI (5′-AAAAGCTAGCTCACTTATCGTCGTCATCCTIGTAATCGCCATACAGGCTGCTTCCACT-3′; SEQ ID NO:15), respectively. The amplified products were subcloned into the XhoI and NheI sites of the mammalian expression vector pCXN2.133, a slightly modified version of pCXN234 with multiple cloning sites. In order to introduce a Flag-tag between aa residues 35 and 36 of the APCDD1 protein, N-terminal region of the APCDD1 was PCR-amplified using the forward primer (APCDD1-FXhoI) and a reverse primer (APCDD1-R-Flag-AvrII: 5′-AAAACCTAGGCTTATCGTCGTCATCCTTGTAATCATGAGACCTGCTGTCTGGAT-3′; SEQ ID NO:16), which was followed by digestion with restriction enzymes XhoI and AvrII. The C-terminal region of the APCDD1 and the truncated APCDD1 proteins with the C-terminal HA-tag was obtained through digestion of the pCXN2.1-WT-APCDD1-HA and the pCXN2.1-APCDD1-ΔTM-HA constructs with restriction enzymes AvrII and NheI. These two fragments were ligated with AvrII site, and subsequently subcloned into the XhoI and NheI sites of the pCXN2.1 vector. In order to generate expression constructs for N-terminal GFP-tagged APCDD1 protein, the coding region of the APCDD1 and the rabbit b-globin 3′-flanking sequences were cut out from the pCXN2.1-APCDD1 constructs with restriction enzymes XhoI and BamHI, and subcloned in frame into the XhoI and BamHI sites of pEGFP-C1 vector (Clontech). The templates were also subcloned into the XhoI and BamHI sites of pBluescript-SK (−) vector (Stratagene). In order to express the GST fusion APCDD1 protein in bacteria, the extracellular domain of the human APCDD1 (aa residues 28-486) was PCR-amplified using the following primers: APCDD1-F-EcoRI (5′-AAAAGAATTCCCTTCATCCAGACAGCAGGTC-3′; SEQ ID NO:17) and APCDD1-ΔTM-R-XhoI (5′-AAAACTCGAGTCAGCCATACAGGCTGCTTCCACT-3′; SEQ ID NO:18). The amplified fragment was subcloned in-frame into the EcoRI and XhoI sites of pGEX-4T-3 vector (GE Healthcare Life Sciences). pGEM Wnt8 (from R. Harland, U. C. Berkley), the Sia luciferase reporter gene (from D. Kimmelman, U. Washington), and pSP36 β-catenin (from B. M. Gumbiner, U. Virginia) have been previously described.

To generate a Xenopus expression vector for Xenopus APCDD1, we used a full length cDNA clone (BC080377, from Open Biosystems) as template and amplified the open reading frame with the following primers: Sense: 5′-CCATCGATGGCCACCATGGGAATCCCCTGTTTCTACTGC-3′ (SEQ ID NO:19), and Antisense: 5′-CCACCGTCGACGCTTAGAATTTCCAAATGTAGCAAAG-3′ (SEQ ID NO:20). The PCR product was inserted as a ClaI/SalI fragment in CS2+2XHA, resulting in CS2+XAPCDD1 HA.

The full length mouse APCDD1 cDNA was amplified by RT-PCR from brain endothelial cells using the First Strand Synthesis Kit and High Fidelity Amplification Kit (Roche Applied Science) with the following primers: APCDD1F (5′-GGGGACAGAGACGGACTACA-3′; SEQ ID NO:21), and APCDD1R (5′-CAAGGCATTCAAGTGCATC-3′; SEQ ID NO:22). The amplified cDNA was confirmed by sequencing and subcloned into PCRII TOPO and pCAGGS vectors for in vitro transcription and chick neural tube electroporations, respectively. The APCDD1ΔTM isoform containing the extracellular domain of mouse APCDD1 (aa 1-486) was amplified by PCR from the full length cDNA using the following primers: APCDD1F (5′-GGGGACAGAGACGGACTACA-3′; SEQ ID NO:23) and APCDD1ΔTM (5′-CTGCCCTGCCTGCCATACAGATGACCTTGACTGTC-3′; SEQ ID NO:24) and subcloned into pCAGGS vector for chick electroporation.

To generate the expression construct for the human WNT3A (pCXN2.1-WNT3A), PCR was performed using cDNA from plucked human hairs and the following primers: WNT3A-F-XhoI (5′-AAAACTCGAGCGGCGATGGCCCCACTCGGATACTT-3′; SEQ ID NO:25), WNT3A-R-NheI (5′-AAAAGCTAGCCTACTTGCAGGTGTGCACGTCGT-3′; SEQ ID NO:26). For the C-terminal HAtagged human WNT3A (pCXN2.1-WNT3A-HA), the following reverse primer was used: WNT3A-R-HA-NheI (5-AAAAGCTAGCTAGGCGTAGTCGGGCACGTCGTAGGGGTACTTGCAGGTGTGCACGTCGT-3′; SEQ ID NO:27). To generate the expression construct for the extracellular domain of the human CD40 with the C-terminal HA tag (aa residues 1-193; pCXN2.1-CD40-EC-HA), PCR was performed using human thymus cDNA as a template and the following primers: CD40-F-XhoI (5′-ATATCTCGAGCCTCGCTATGGTTCGTCTGCCT-3′; SEQ ID NO:28) and CD40-R-HA-NheI (5′-ATATGCTAGCTAGGCGTAGTCGGGCACGTCGTAGGGGTATCTCAGCCGATCCTGGGGA-3′; SEQ ID NO:29). To generate the construct for the extracellular domain of the human LRP5 with the C-terminal Flag tag (aa residues 1-1384; pCXN2.1-LRP5-EC-Flag), the N-terminal sequences of the human LRP were PCR-amplified using the expression construct for the full-length human LRP5 (kindly provided by Dr. Patricia Ducy in Columbia University) as a template and the following primers: LRP5-F-EcoRI (5-AAAAGAATTCCGGACAACATGGAGGCAG-3 3′; SEQ ID NO:30) and LRP5-R-Flag-NheI (5′-AAAAGCTAGCTACTTATCGTCGTCATCCTTGTAATCGCTGTGGGCCGGGCTGTCGTCTGA-3′; SEQ ID NO:31). The amplified products were subcloned into the XhoI/NheI sites (for WNT3A and CD40) or EcoRI/NheI sites (for LRP5) of the pCXN2.1 vector. To generate the expression construct for the mouse Frizzled 2 (mFzd2), the full-length open reading frame of the mFzd2 was purchased from Invitrogen (clone ID 6411627), which was subcloned into the NotI sites of the pCXN2.1 vector.

Chick neural tube electroporations. The full length mouse APCDD1 or APCDD1ΔTM isoform was transfected into the chick neural tube (stage 12-13) using in ovo electroporation as described (Briscoe, J. et al. (2000) Cell 101, 435-445). The chick embryos were grown for 3-4 additional days in the 39° C. incubator, fixed with 4% PFA/0.1M phosphate buffer, washed and cryoprotected as described (Briscoe, J. et al. (2000) Cell 101, 435-445) before being processed for in situ hybridization or immunofluorescence.

Cell counts and statistical analysis. Spinal cord Sox3+ progenitors, Isl1/2+ ventral motor neurons, Isl1/2+ dorsal interneurons and Chx10+ V2a interneurons were counted from 8 independent 12 um thick sections of chick spinal cord from each transfected embryo. The nucleus stained with the transcription factor was considered one cell for this purpose. The cells were counted from both the electroporated side and the opposite control side. The plots were created using Sigma plot with values representing the mean for each embryo. Statistical significance was determined using the Student t-test.

Chick neural tube electroporations. The full length WT-APCDD1, L9R APCDD1, L9V APCDD1, mouse APCDD1 or mAPCDD1_TM isoform were subcloned into the pCAGGS vector and transfected into the chick neural tube (stage 12-13) together with nGFP vector (pCIG) using in ovo electroporation. The chick embryos were grown for 3-4 additional days in the 39C incubator, fixed with 4% PFA/0.1M phosphate buffer, washed and cryoprotected as described 35 before being processed for in situ hybridization or immunofluorescence. For the Wnt reporter assays, the TOP::eGFP reporter (M38 TOP::eGFP from Addgene) was transfected alone or in combination with WT-APCDD1 or L9R-APCDD1. The chick embryos were grown for 12 hours in the 39° C. incubator, fixed with 4% PFA/0.1M phosphate buffer for 30 minutes, washed and cryoprotected as described before being processed for immunofluorescence.

Cell counts and statistical analysis. Spinal cord Sox3+ progenitors, Isl1/2+ ventral motor neurons, Isl1/2+ dorsal interneurons, and Chx10+ V2a interneurons were counted from 8 independent 12 μm thick sections of chick spinal cord from each transfected embryo. The nucleus stained with the transcription factor was considered one cell for this purpose. The cells were counted from both the electroporated side and the opposite control side. The plots were created using Sigma plot with values representing the mean for each embryo. Statistical significance was determined using the Student t-test.

Transient transfections and western blots in cultured cells and human scalp skin. HEK293T cells were plated in 6 well dishes the day before transfection. Expression plasmids of APCDD1 were transfected with FuGENE® 6 (Roche Applied Science) at 60% confluency. Total amount of transfected plasmids were adjusted with the empty pCXN2.1 vector. The cells were cultured 48 h after transfection in Opti-MEM (GIBCO). The cells were harvested and homogenized by sonication in 25 mM HEPES-NaOH (pH 7.4), 10 mM MgCl2, 250 mM Sucrose, and 1× Complete Mini Protease Inhibitor Cocktail (Roche Applied Science). The cell debris was removed by centrifugation at 3,000 rpm for 10 min at 4° C., and the supernatant was collected as cell lysates. The cultured medium with 1× Complete Mini Protease Inhibitor Cocktail was centrifuged at 1,500 rpm for 5 min at 4° C. The supernatant was purified with 0.2 μm syringe filters (Thermo Fisher Science), and concentrated using Amicon Ultra-15 Centrifugal Filter Unit with Ultracel-10 Membrane (Millipore) according to the manufacturer's recommendations. To examine the N-glycosylation of APCDD1, the total cell lysates from the wild-type APCDD1 expressing cells were treated with PNGase F (Sigma) following the manufacturer's recommendations. Total cell lysates from human scalp skin were extracted by homogenization in 50 mM Tris-HCl (pH 8.0), 150 mM NaCl, 1.0% NP40, 0.5% sodium deoxycholate, 0.1% SDS, and 1× Complete Mini Protease Inhibitor Cocktail. All samples were mixed with equal amount of Laemmli Sample Buffer (Bio-Rad Laboratories) containing 5% β-mercaptoethanol, boiled at 95° C. for 5 min, and analyzed by 10% SDS-polyacrylamide gel electrophoresis (SDS-PAGE). Western blots were performed as described previously (Bazzi, H., et al. (2006) Differentiation 74, 129-140). The primary antibodies used were rabbit polyclonal anti-HA (diluted 1:4,000; Abcam), rabbit polyclonal anti-APCDD1 (1:20,000), mouse polyclonal anti-APCDD1 (1:1,000; Abnova), rabbit polyclonal anti-GFP (1:1,000; Invitrogen), mouse monoclonal anti-Flag M2 (1:1,000; Sigma), and rabbit polyclonal anti-β-actin (1:10,000; Sigma).

Wnt reporter assays in HEK293T cells. HEK293T cells were seeded in 12 well dishes the day before transfection. Either 100 ng of TOPFlash (active) or FOPFlash (inactive) Wnt reporter vector was transfected into each well along with constructs for WNT3A (200 ng), Fzd2 (100 ng), LRP5 (100 ng), and/or wild type APCDD1-HA (800 ng) using Lipofectamine 2000 (Invitrogen). A construct for galactosidase reporter (100 ng) was also transfected for normalization of transfection efficiency. The cells were lysed 36 h after transfection and the signals were assayed using the appropriate substrates for luciferase (Steady-Glo Luciferase Assay System) and β-galactosidase (Promega) on a 20/20n luminometer (Turner Biosystems) for luciferase and Model 680 microplate reader (BioRad) for β-galactosidase. The Wnt activity was measured based on the ratio of TOP/FOP luciferase activity. The results represent triplicate determination of a single experiment that is representative a total of five similar experiments.

Co-Immunoprecipitation (Co-IP) assays. Expression plasmids (total 4 μg) were transfected into HEK293T cells seeded on 60 mm dishes with FuGENE® 6 (Roche Applied Science) at 60% confluency. 24 h after the transfection, the cells were harvested and homogenized in lysis buffer (20 mM Tris-HCl (pH 7.5), 137 mM NaCl, 10% Glycerol, 2 mM EDTA, 0.5% Triton X, and 1× Complete Mini Protease Inhibitor Cocktail). Total cell lysates were collected by centrifugation at 14,000 rpm for 15 min at 4° C. The samples were incubated with either mouse monoclonal anti-Flag M2 agarose gel (Sigma) or mouse monoclonal anti-HA agarose gel (Sigma) for 3 h at 4° C. The agarose beads were washed with lysis buffer for five times. The precipitated proteins were eluted with NuPAGE® LDS Sample Buffer containing Sample Reducing Agent (Invitrogen), incubated at 75° C. for 10 min, and separated on 10% NuPAGE® gels (Invitrogen). Western blots were performed using rabbit polyclonal anti-HA (diluted 1:4,000; Abcam) or mouse monoclonal anti-Flag M2 antibody (1:1,000; Sigma).

GST pulldown assays. Expression of GST-fusion proteins was induced in DH5a (Invitrogen) by the addition of 0.1 mM isopropyl-β-D-thiogalactopyranoside at 37° C. for 3 h, and the fusion proteins were isolated from bacterial lysates by affinity chromatography with glutathione-Sepharose beads (GE Healthcare Life Sciences). HEK293T cells overexpressing LRP5-EC-Flag, WNT3A-HA, or CD40-EC-HA were dissolved in lysis buffer (20 mM Tris-HCl (pH 7.5), 137 mM NaCl, 10% Glycerol, 2 mM EDTA, 0.1% Triton X, and 1× Complete Mini Protease Inhibitor Cocktail), and centrifuged at 12,000 g at 4° C. for 30 min. Clarified supernatants were incubated in the presence of either GST alone or GSTAPCDD1ΔTM fusion proteins (10 μg) immobilized to glutathione beads at 4° C. for 3 h. After incubation, the beads were washed with the lysis buffer for five times, resuspended in NuPAGE® LDS Sample Buffer containing Sample Reducing Agent (Invitrogen), fractioned by 10% NuPAGE® (Invitrogen), and analyzed by western blotting. The antibodies used were: rabbit polyclonal anti GST (1:3,000; Santa Cruz Biotechnology), rabbit polyclonal anti-HA (1:4,000; Abcam) and mouse monoclonal anti-Flag M2 (1:1,000; Sigma).

In situ hybridization. A part of the human APCDD1 cDNA (SEQ ID NO:1: nt. 338-803) was cloned into pCR®II-TOPO vector (Invitrogen). The antisense and sense DIG-labeled cRNA probes were synthesized from the linearized vectors with T7 and SP6 RNA polymerases (Roche Applied Science), respectively. Dissected human hair follicles were fixed with 4% paraformaldehyde-PBS at 4° C. overnight. After dehydration step with 30% sucrose-PBS, the tissues were frozen in OCT compound and sectioned on glass slides at the thickness of 10 μm. In situ hybridization was performed following the methods described previously with minor modifications (Aoki, N., et al. (2001) J. Invest. Dermatol. 116, 359-365). At the prehybridization steps, the sections were treated with 1 μg/ml Protease K for 15 min at 37° C. Hybridization was performed at 55° C. overnight. In situ hybridizations on chick spinal cord sections were performed as described (Schaeren-Wiemers, N. & Gerfin-Moser, A. (1993) Histochemistry 100, 431-440). The antisense mAPCDD1 mRNA was generated using the In vitro transcription kit (Roche, Indianapolis, Ind.) with T7 RNA polymerase. The antisense chick Sim1 mRNA was generated using the T3 RNA polymerase.

Indirect immunofluorescence (IIF). IIF on cultured cells and fresh frozen sections of individually dissected hair follicles was performed as described previously (Bazzi, H., et al. (2006) Differentiation 74, 129-140). IIF on HEK293T cells were performed 48 h after the APCDD1 expression constructs were transfected. For some stainings, cell membrane was labeled with rhodamine-phalloidin (Invitrogen). The primary antibodies used were mouse polyclonal anti-APCDD1 (diluted 1:1,000; Abnova), rabbit polyclonal anti-APCDD1 (1:4,000), rabbit polyclonal anti-pan-cadherin (1:200; Invitrogen), and goat polyclonal anti-calnexin (1:200; Santa Cruz Biotechnology). Immunofluorescence on chick spinal cord sections was performed as described (Agalliu, D. & Schieren, I. (2009) Neural Dev. 4, 2). The monoclonal antibodies against Nkx2.2, Pax6, Pax7, En-1 and Evx1 were purchased from DSHB (Iowa); rabbit anti Olig2 (Chemicon, Billerica Mass.), rabbit anti-Sox3, rabbit anti Chx10, and guinea pig anti Isl1/2, sheep anti GFP (Biogenesis) and mouse anti β3-tubulin (Tuj1; Covance) were used as described (Agalliu, D. & Schieren, I. (2009) Neural Dev. 4, 2).

Quantitation of subcellular localization of APCDD1 protein. Based on the results of immunofluorescence with rabbit polyclonal anti-APCDD1 antibody in HEK293T cells transfected with APCDD1-expression constructs, we measured the subcellular localization of APCDD1 proteins. The cell outline was visualized using rhodamine-phalloidin (Invitrogen). Images were processed in Image J, splitting the channels. First, the outline of the cell was used to measure the signal within the whole cell. Second, scaling the cell frame down, the signal inside the cell was measured. For each cell, the following values were recorded: 1) the adjusted total signal in the cell (the level of fluorescence, relative to the background); 2) the adjusted signal inside the cell; 3) the adjusted signal in the membrane; and 4) the ratio between adjusted signal in the membrane and inside the cell. The adjusted signal (Sadj) was calculated by subtracting the background signal and then normalizing to the background (B) signal levels in an empty area of equal size within the same image. Sadj=(S−B)/B. For example, a reported signal Sadj=5 indicates 5 times stronger than the background 40,41. Data are represented as average+/−SEM (standard error of the mean). P values are reported using heteroscedastic 2-tailed t-tests, applying the Bonferroni correction to take into account the 3-way comparisons (WT versus L9R; WT versus WT+L9R; L9R versus WT+L9R). All reported values are measured in 20 cells per condition.

Xenopus embryo manipulations. Xenopus laevis embryos were obtained by in vitro fertilization, cultured in 0.1×MMR and staged according to Nieuwkoop and Faber (Nieuwkoop, P. D. & Faber, J. (1967) Normal table of Xenopus laevis. North Holland Publishing Co. Amsterdam, The Netherlands). APCDD1 RNA was produced from the pBluescript-SK (−)-human APCDD1 construct using the mMessage Machine in vitro T7 transcription kit (Ambion). For full length XAPCDD1 RNA expression, the vector (pCMV-SPORT6) was restricted with XbaI and transcribed with the mMessage Machine in vitro SP6 transcription kit (Ambion). For XAPCDD1 HA expression, CS2+XAPCDD1 HA was restricted with NotI and transcribed with the same kit. RNA and reporter DNA injections were done at the 4 cell stage. For the effect of APCDD1 on antero-posterior patterning, APCDD1 RNA (1 ng) was injected in the marginal zone of both dorsal blastomeres at 4 cell stage. For the ventral effect of APCDD1, one ventral blastomere was injected in the marginal zone at the 4 cell stage (Vonica, A. & Gumbiner, B. M. (2002) Dev. Biol. 250, 112-127).

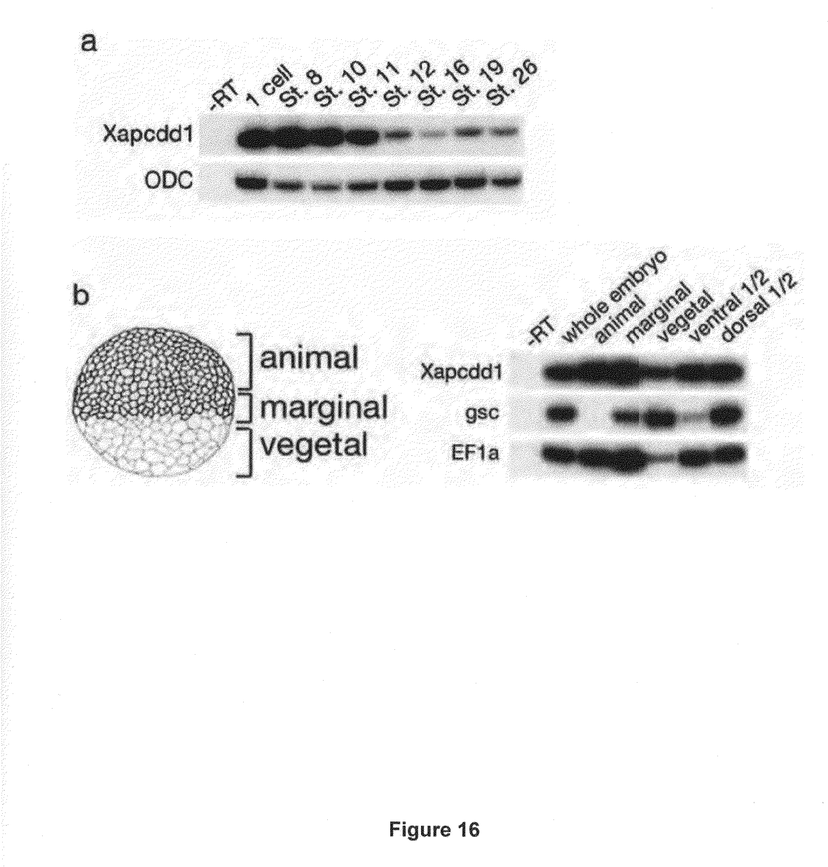

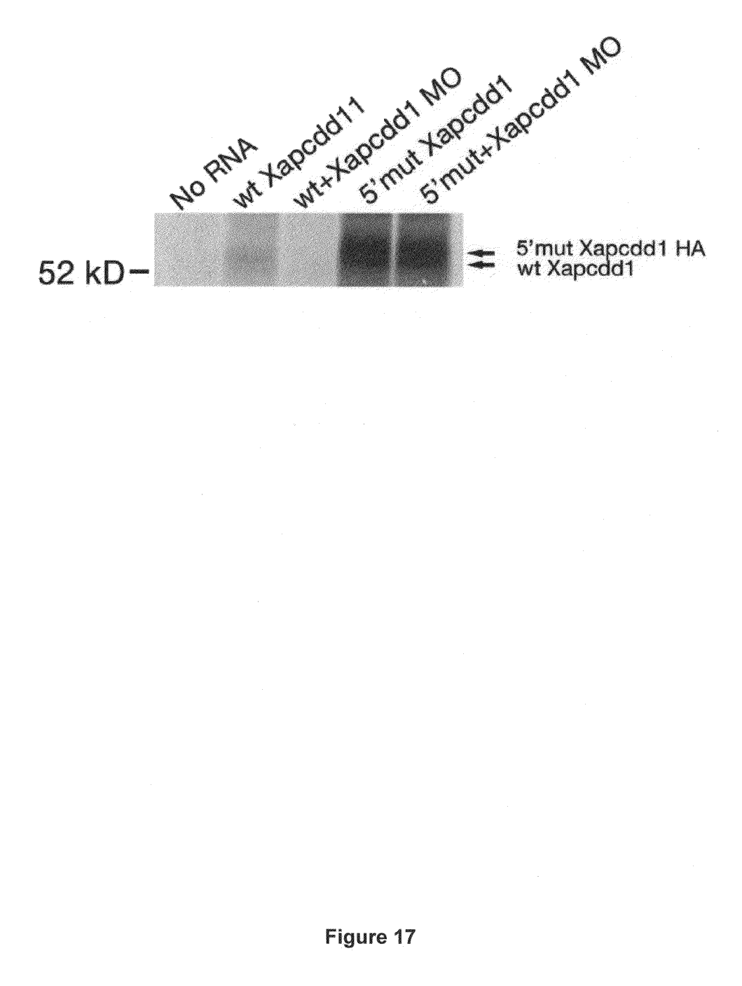

Morpholino oligonucleotide techniques. Translation blocking MO oligonucleotide (AS1 MO) was designed and synthesized by GeneTools, with the sequence: 5′-TGGTAGTTCAGCTCCAGAATGTCCT (SEQ ID NO:32), where the nucleotide in bold is the first one in the open reading frame. The efficiency and specificity of the MO was tested on the full length mRNA (WT APCDD1 in FIG. 16) and the XAPCDD1 HA mRNA lacking the 5′ UTR to which AS1 MO binds (5′ mut APCDD1 in FIG. 16). RNA preincubation with MO and in vitro translation were performed as described previously42, using the Promega Reticulocyte Lysate System for translation in the presence of ^{[S35]}Met. MO was injected at the 4 cell stage in both dorsal blastomeres (30 ng), alone or together with XAPCDD1 RNA (300 pg) or DNWnt8 RNA (300 pg). Embryos were scored for the dorso-anterior index (DAI) at stage 41.

Transcription assays. Injected embryos were collected at stage 9 and processed for luciferase assays (Promega) as described (Vonica, A. & Gumbiner, B. M. (2002) Dev. Biol. 250, 112-127). The Sia reporter gene (Smith, J. C., Price et al. (1991) Cell 67, 79-87) was injected at 100 pg DNA. All assays were in triplicate, and each experiment was repeated three times.

RT-PCR in Xenopus embryos. Radioactive RT-PCR was performed as described previously (Wilson, P. A. & Melton, D. A. (1994) Curr. Biol. 4, 676-686). mRNA was purified from whole embryos, or from fragments cut with a hair knife, at the indicated stages with RNA-Bee (Tel-Test, Inc.), before reverse transcription with SuperScript III (Invitrogen), using Poly dT as priming oligonucleotide. The primers used for PCR were: ODC: sense: 5′-CGAAGGCTAAAGTTGCAG-3′ (SEQ ID NO:33), antisense: 5′-AATGGATTTCAGAGACCA-3′ (SEQ ID NO:34); goosecoid (gsc): sense: 5′-TCTTATTCCAGAGGAACC-3′ (SEQ ID NO:35), antisense: 5′-ACAACTGGAAGCACTGGA-3′ (SEQ ID NO:36); XAPCDD1 sense: 5′-CTGGAGCTGAACTACCATGG-3′ (SEQ ID NO:37), antisense: 5′-TGACCCTCGATGTTTGGAGGC-3′ (SEQ ID NO:38).

Western Blot in Xenopus embryos. Xenopus embryos were injected at the 4 cell stage with 1 ng RNA of WT human APCDD1 alone, or together with L9R mutant RNA (1 ng). Injections also contained 1 ng LacZ RNA as loading control. Embryos were retrieved at stage 10, homogenized in NP-40 extract buffer, mixed with LDS sample buffer and run on NuPage 4-12% gels (Invitrogen). After transfer to PVDF membrane, blots were incubated with anti-APCDD1 (1:10,000) or anti-β-Galactosidase (1:1000; ProSci Inc.) antibodies, and stained with ECL Western Blotting Reagent (GE Healthcare).

### Results

Hair follicle (HF) miniaturization is a degenerative process that proportionally reduces the dimensions of the epithelial and mesenchymal compartments, and leads to the conversion of thick, terminal hair to fine, vellus hair. Despite molecular characterization of several genes differentially expressed among HF cell populations, family-based linkage approaches in AGA5, and recent genome-wide association studies could not elucidate the genetic basis of this disorder.

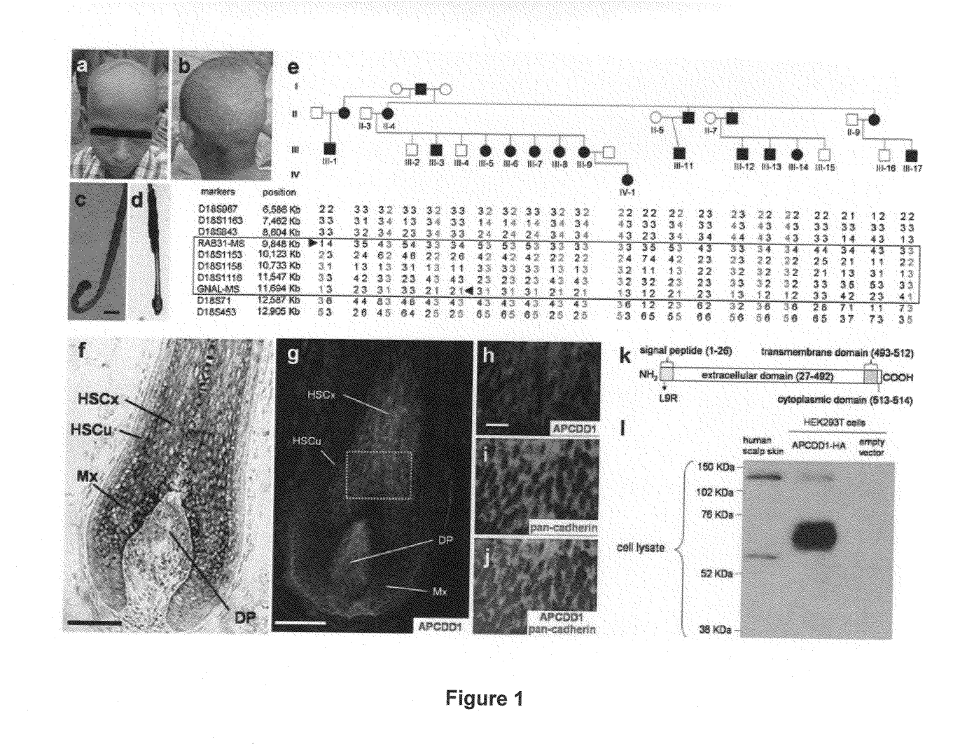

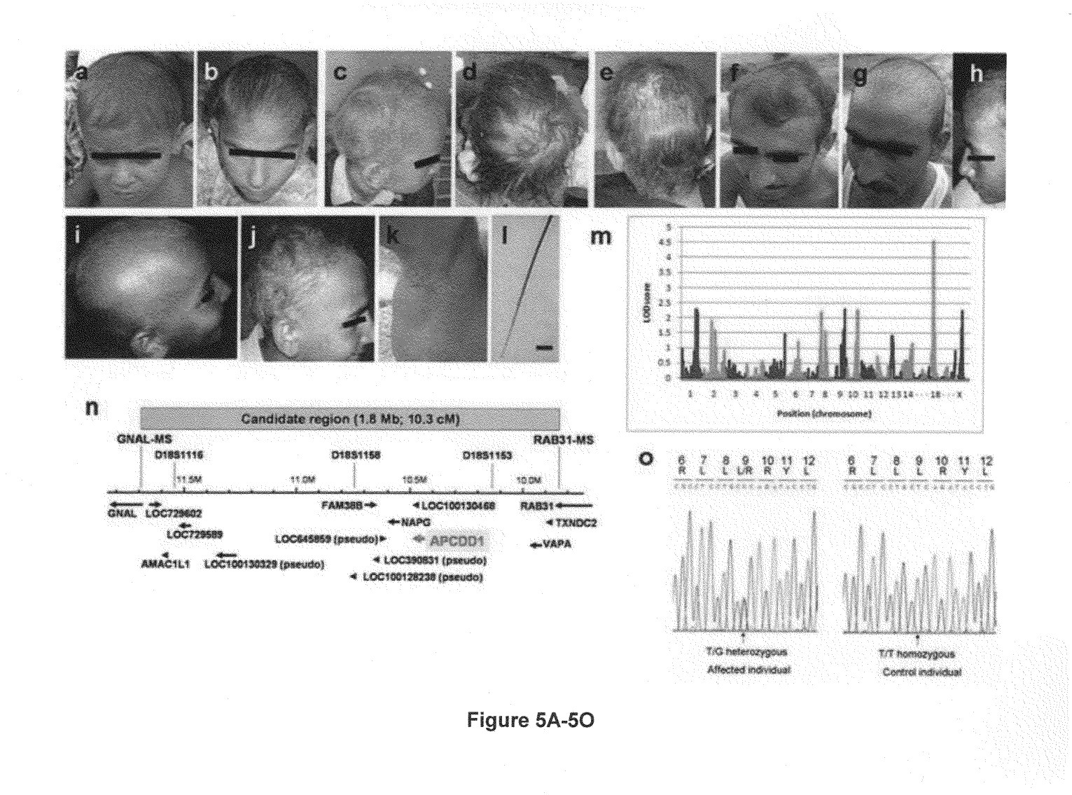

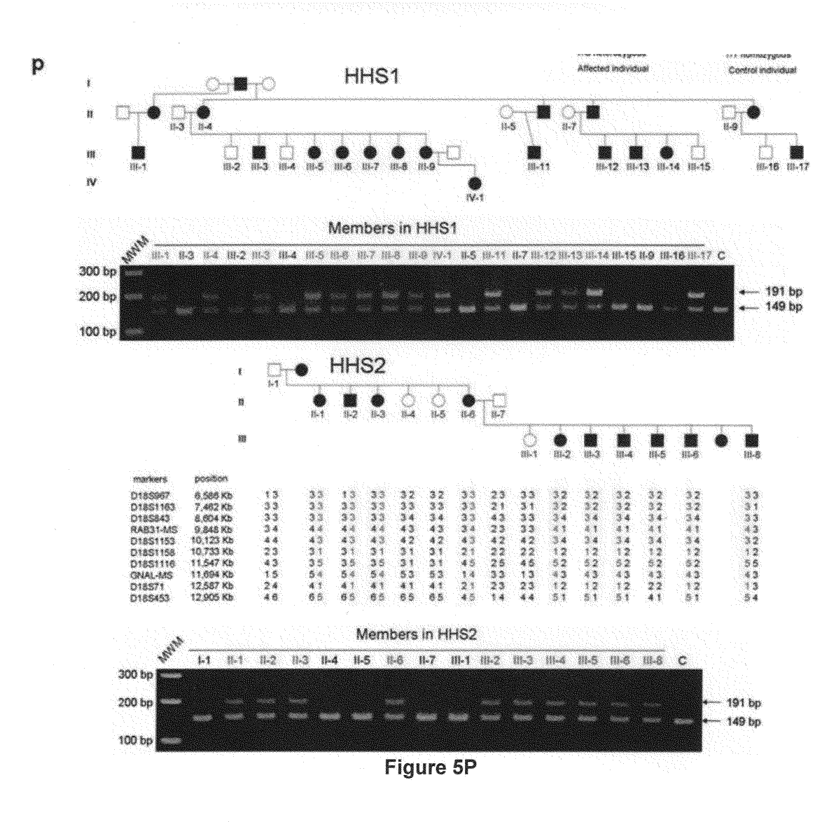

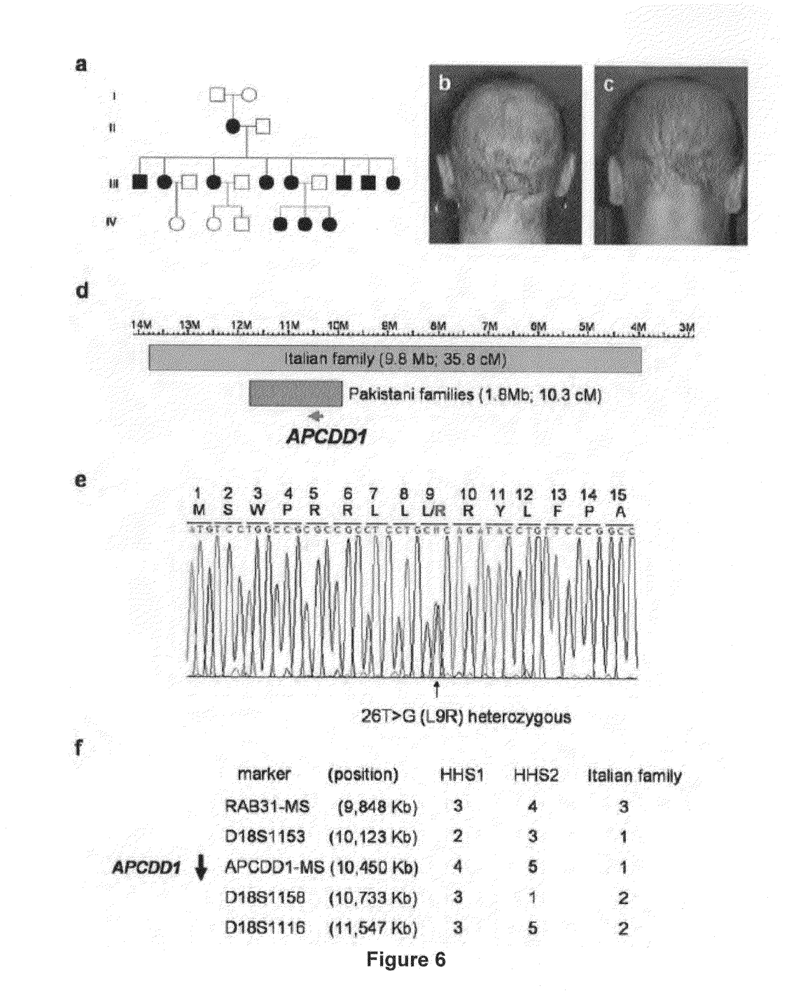

To gain insight into the genetic underpinning of HF miniaturization in humans, we focused on identifying the causative gene of autosomal dominant hereditary hypotrichosis simplex (HHS; OMIM 146520). The disease is characterized histologically by HF miniaturization and a progressive hair loss beginning in childhood, independent of hormonal effects. We performed a genetic linkage study in two large Pakistani families (HHS1 and HHS2) with autosomal dominant HHS (FIG. 1a-d, FIG. 5a-l). We used human mapping arrays with low density (Affymetrix 10K) to genotype 16 and 12 members of each family, respectively. Parametric linkage analysis performed under a dominant model yielded a maximum LOD score of z=4.6 for a haplotype on chromosome 18p11.22 (FIG. 5m). The 2LOD interval spanned from 7.4 Mb to 25 Mb. Genotyping with microsatellite markers enabled us to define the candidate region to 1.8 Mb between the markers RAB31-MS and GNAL-MS (FIG. 1e and FIG. 5n), which contained 8 known genes, 4 pseudogenes and 3 predicted transcripts (FIG. 5n). Direct sequencing analysis of all known genes in the region identified a single heterozygous mutation 26T>G (L9R) in the signal peptide sequence of the Adenomatosis Polyposis Coli Down-regulated 1 (APCDD1) gene (FIG. 5o), described initially as being downregulated by the tumor suppressor APC. The mutation L9R cosegregated with the disease phenotype in both families, was absent in 200 unrelated healthy control Pakistani individuals and in the SNP databases, arguing against it being a polymorphism (FIG. 5p). In addition, we have also identified the identical heterozygous mutation 26T>G (L9R) in the APCDD1 gene in an Italian family with autosomal dominant HHS that was reported previously (FIG. 6), providing independent genetic evidence in support of this finding.

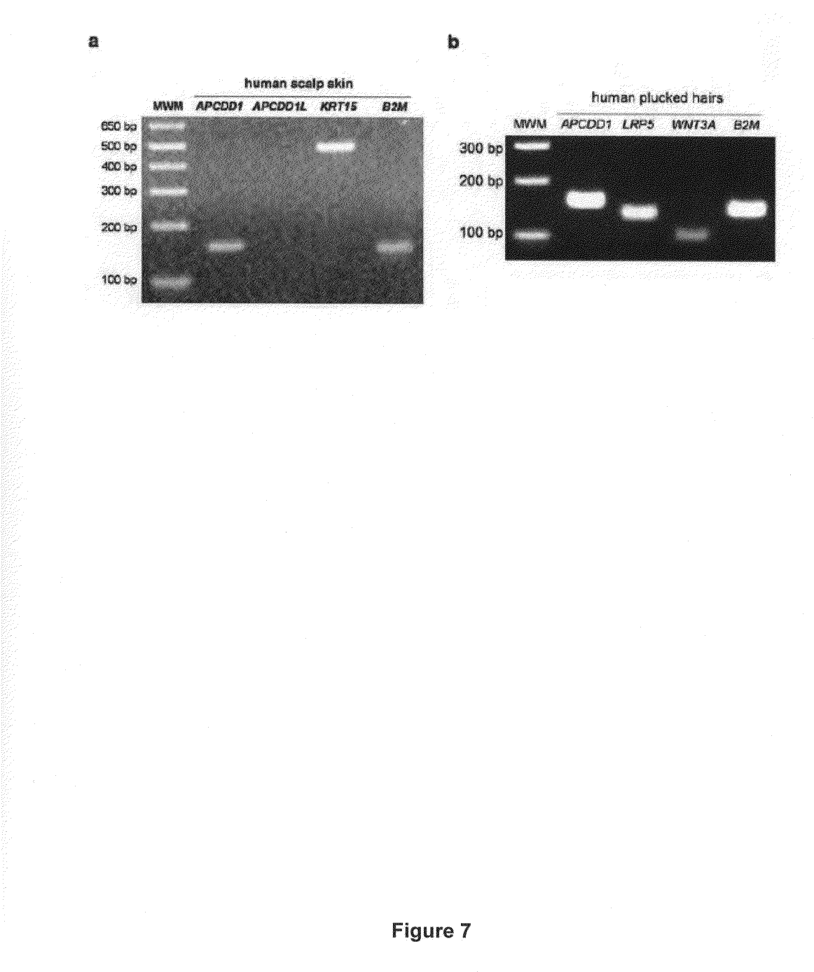

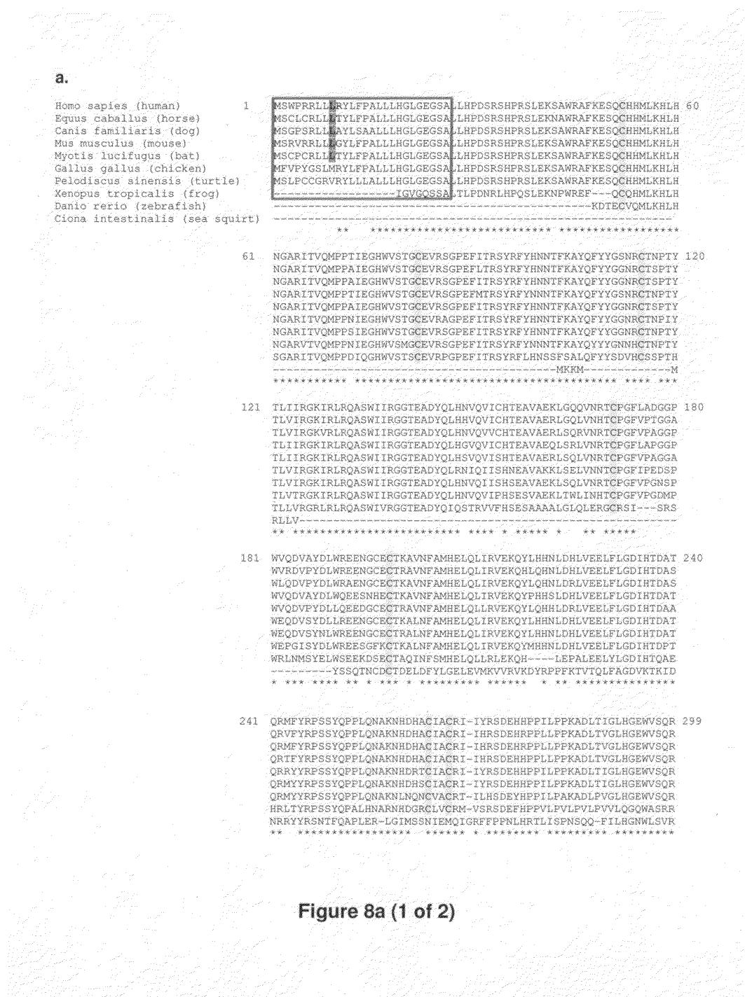

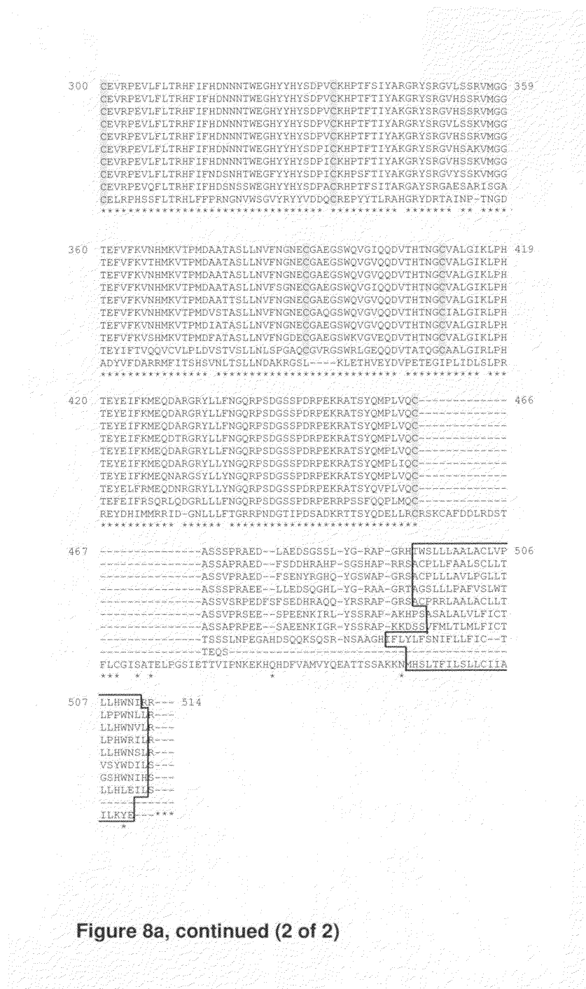

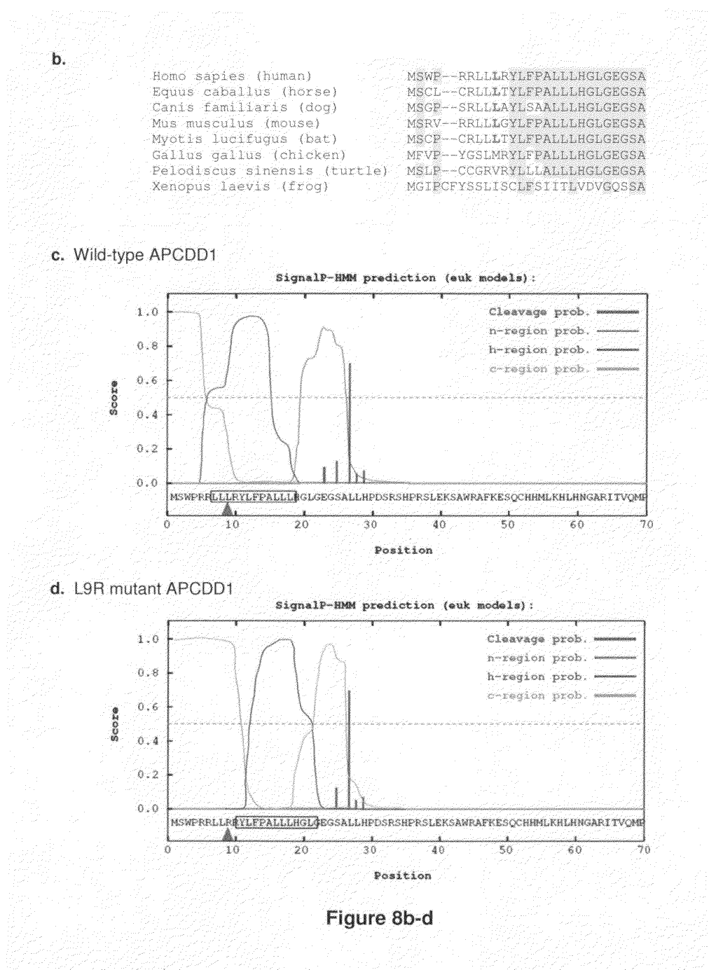

APCDD1 was abundantly expressed in both the epidermal and dermal compartments of the human HF, consistent with a role in HF miniaturization. APCDD1 mRNA and protein were present in human scalp skin by RT-PCR (FIG. 7), and a western blot using an APCDD1 antibody (FIG. 1l). APCDD1 mRNA and protein were also highly expressed in the HF dermal papilla (DP), the matrix, and the hair shaft (FIG. 1f-j). APCDD1 orthologs are conserved throughout evolution (FIG. 8) suggesting that a role in mouse and human HF growth emerged recently in mammalian species.

The similarity in expression pattern with another Wnt inhibitor Wise, and the presence of other Wnt inhibitors in the HF (e.g. Dkk4) led us to postulate that APCDD1 may function as an inhibitor of Wnt signaling in a negative feedback loop. It is noteworthy that APCDD1 contains 12 highly conserved cysteine residues (FIG. 9a), a structural feature that is present in many inhibitors of Wnt signaling and is important for interaction with Wnt ligands or their receptors.

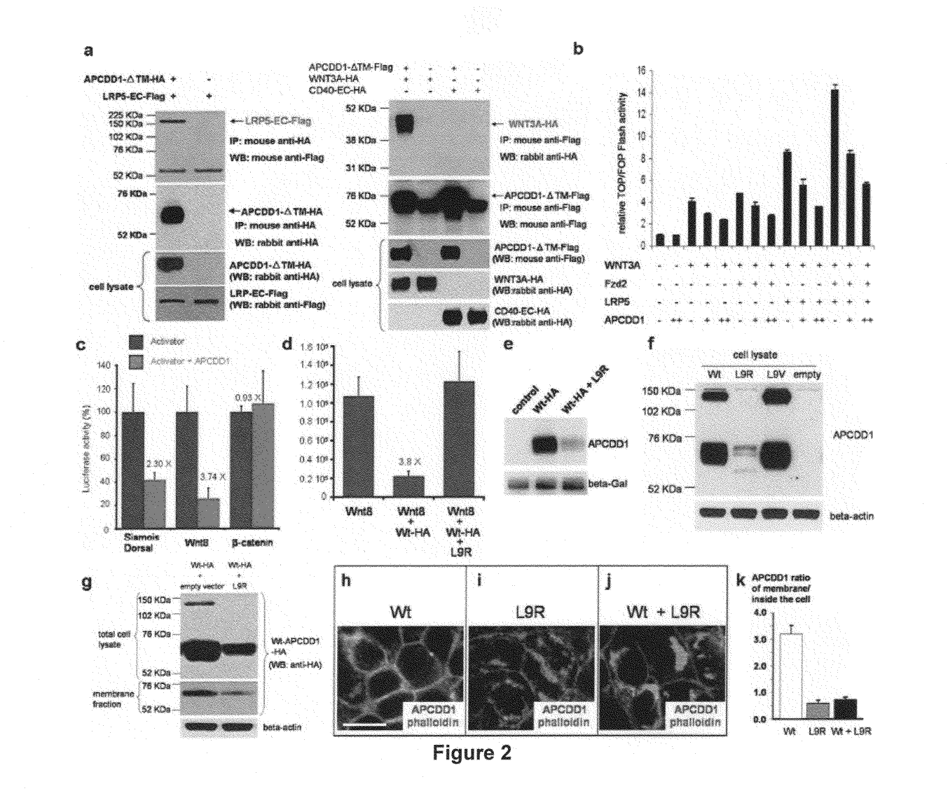

To test if APCDD1 is an inhibitor of Wnt signaling, we first determined if APCDD1 interacts with ligands and receptors of the canonical Wnt pathway. No interaction was found with Fzd2, Fzd8, and Dkk4. In contrast, the extracellular domain of APCDD1 (APCDD1ΔTM) coprecipitated with recombinant tagged forms of Wnt3A and LRP5 (FIG. 2a and FIG. 9a), two proteins important for HF induction (FIG. 7), suggesting that APCDD1 can modulate the Wnt pathway via interaction with both WNT3A and LRP5 at the cell surface. To determine the effect of APCDD1 on Wnt/β-catenin signaling, we performed TOP/FOPFlash Wnt reporter assays in HEK293T cells. Reporter activity induced by WNT3A alone, or in combination with LRP5/Fzd2, was downregulated ˜2-fold by APCDD1 (FIG. 2b), indicating that APCDD1 inhibits the Wnt/β-catenin pathway.

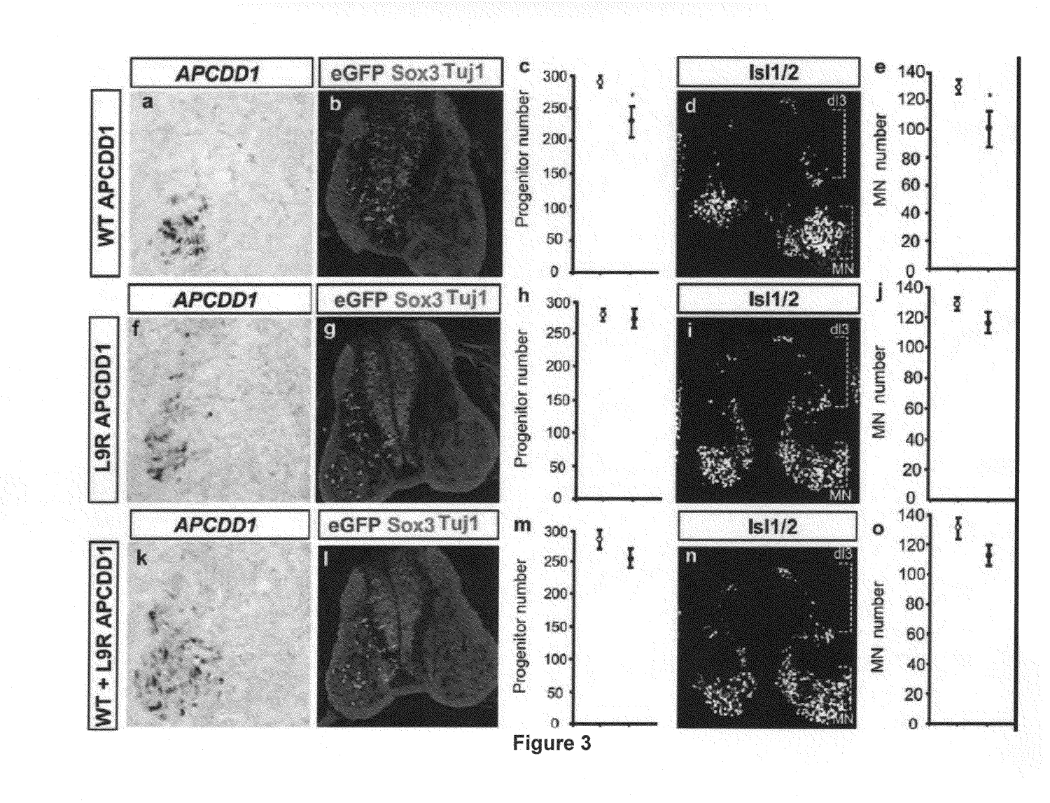

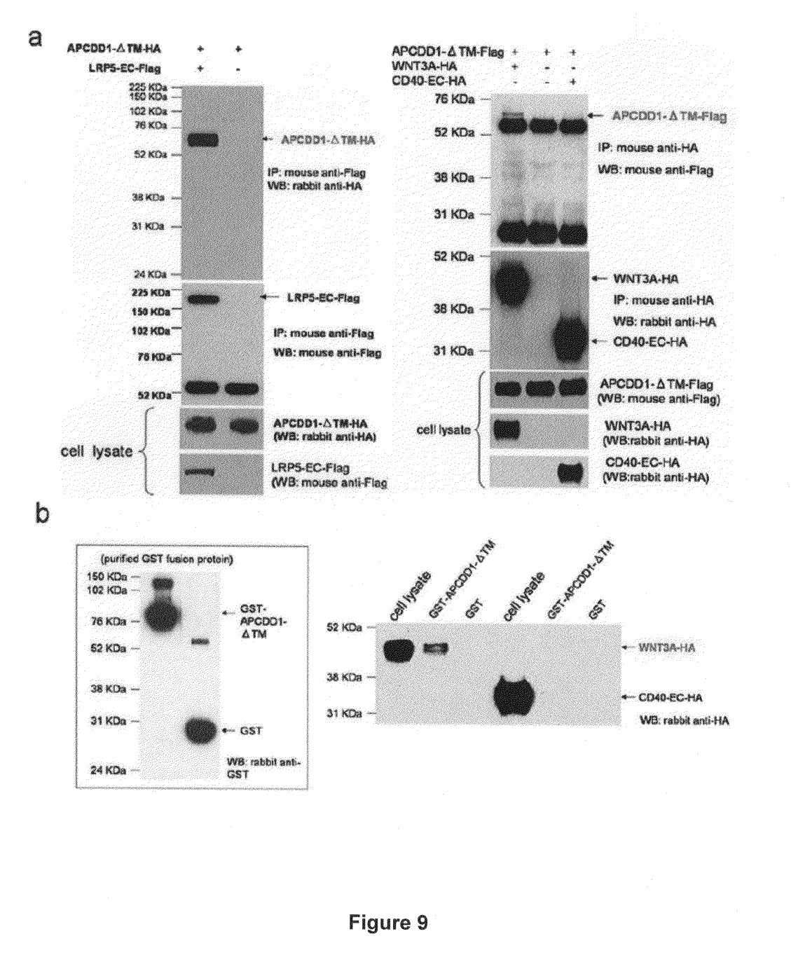

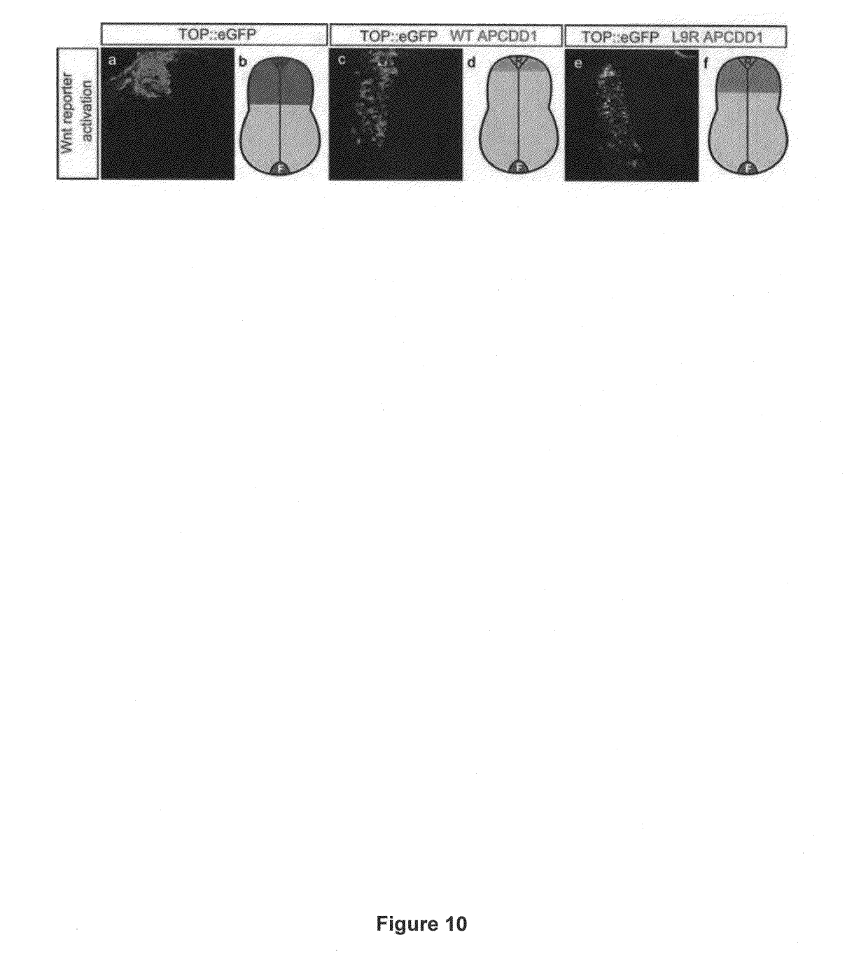

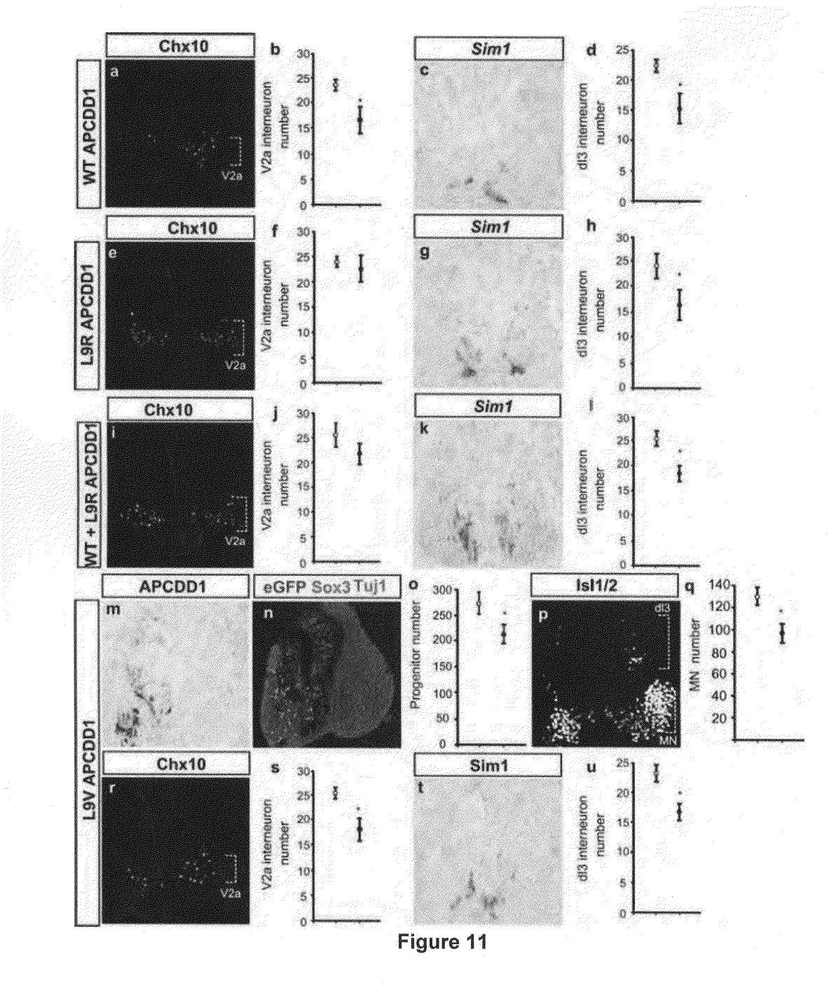

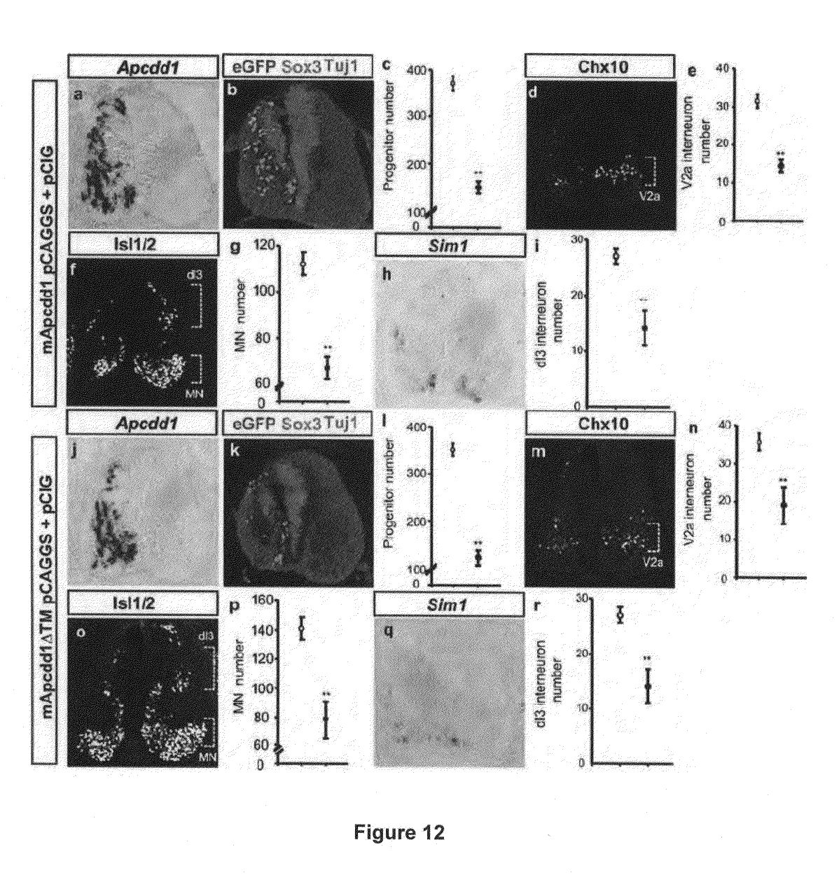

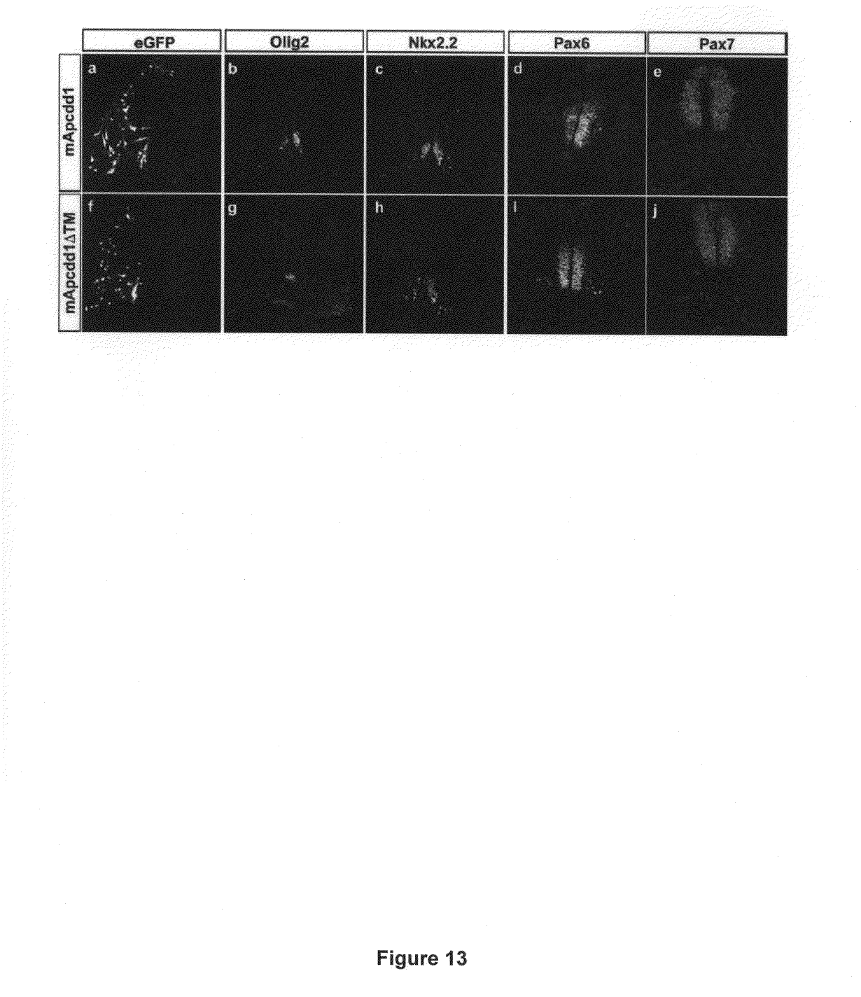

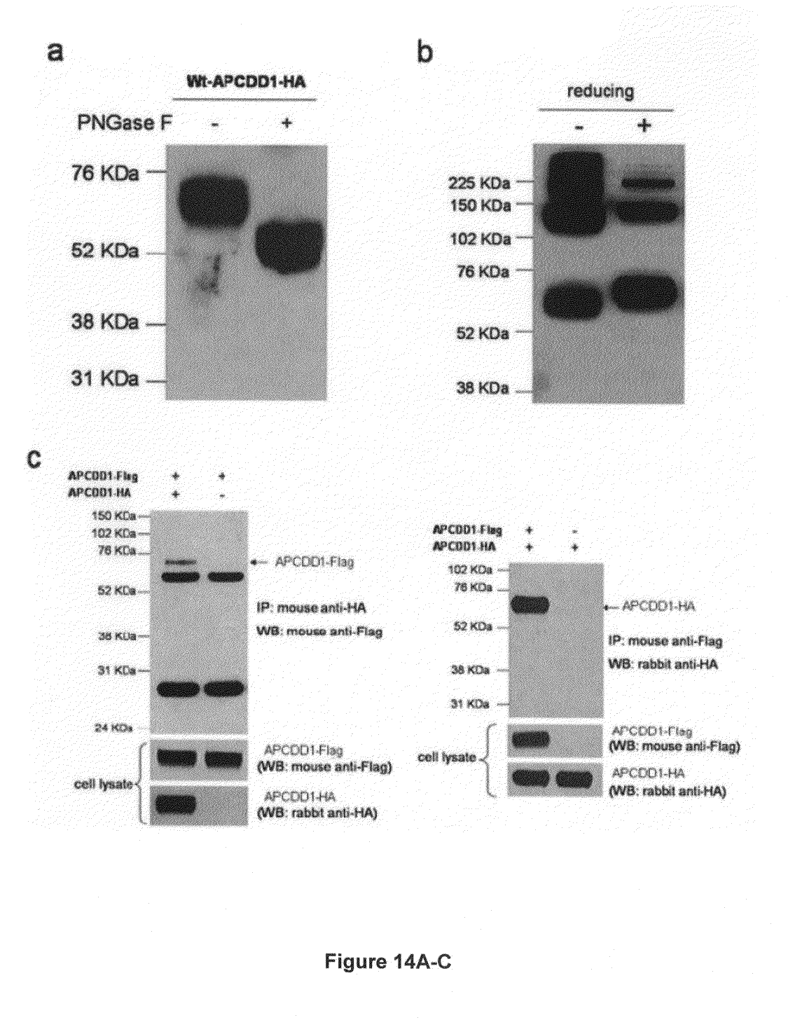

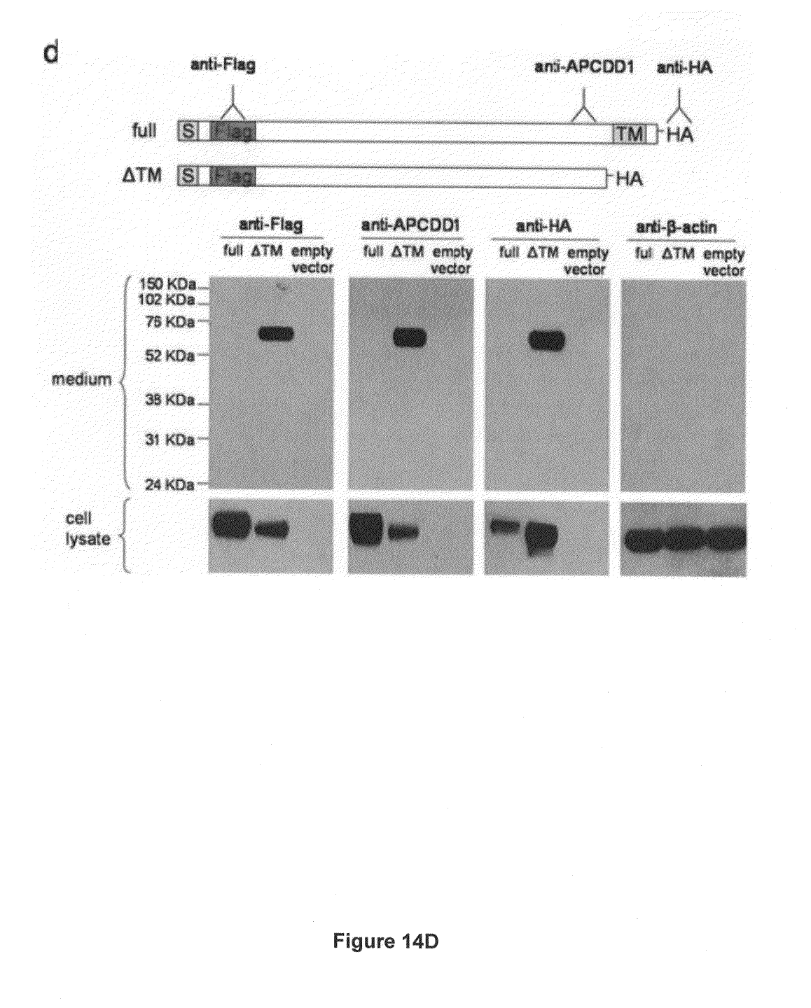

To determine if APCDD1 can function as a Wnt inhibitor in vivo, we selected two systems where the role of Wnt/β-catenin signaling pathway has been well-defined: the generation of neuronal subtypes in the developing spinal cord (Megason, S. G. & McMahon, A. P. (2002) Development 129, 2087-2098; Lei, Q., et al. (2006) Dev. Cell 11, 325-337; Yu, W., et al. (2008) Development 135, 3687-3696), as well as axis specification in the frog (Leyns, L et al. (1997) Cell 88, 747-756; Wang, S., et al. (1997) Cell 88, 757-766). In the chick spinal cord, a dorsal^{high} to ventral^{low} gradient of the Wnt/β-catenin activity promotes neural progenitors proliferation and generation of some neuronal classes. Transfection of the Wnt reporter TOP::eGFP in the chick neural tube revealed a strong activation of the Wnt pathway in the dorsal and intermediate neural precursors as previously shown (FIG. 10). However, expression of APCDD1 strongly reduced eGFP expression levels (FIG. 10), decreased by ˜20-30% the number of Sox3+ neural progenitors, as well as various neuronal subtypes of dorsal and ventral origin (FIG. 3a-e and FIG. 11a-d), the opposite effect to the one observed with overexpression of Wnt pathway activators, see, e.g. Megason, S. G. & McMahon, A. P. (2002) Development 129, 2087-2098; and Lei, Q., et al. (2006) Dev. Cell 11, 325-337. This effect was even stronger with mouse APCDD1, a closer homolog of the chick protein (FIGS. 12a-k and FIG. 13a-e). These findings are consistent with the hypothesis that APCDD1 functions as a Wnt inhibitor.

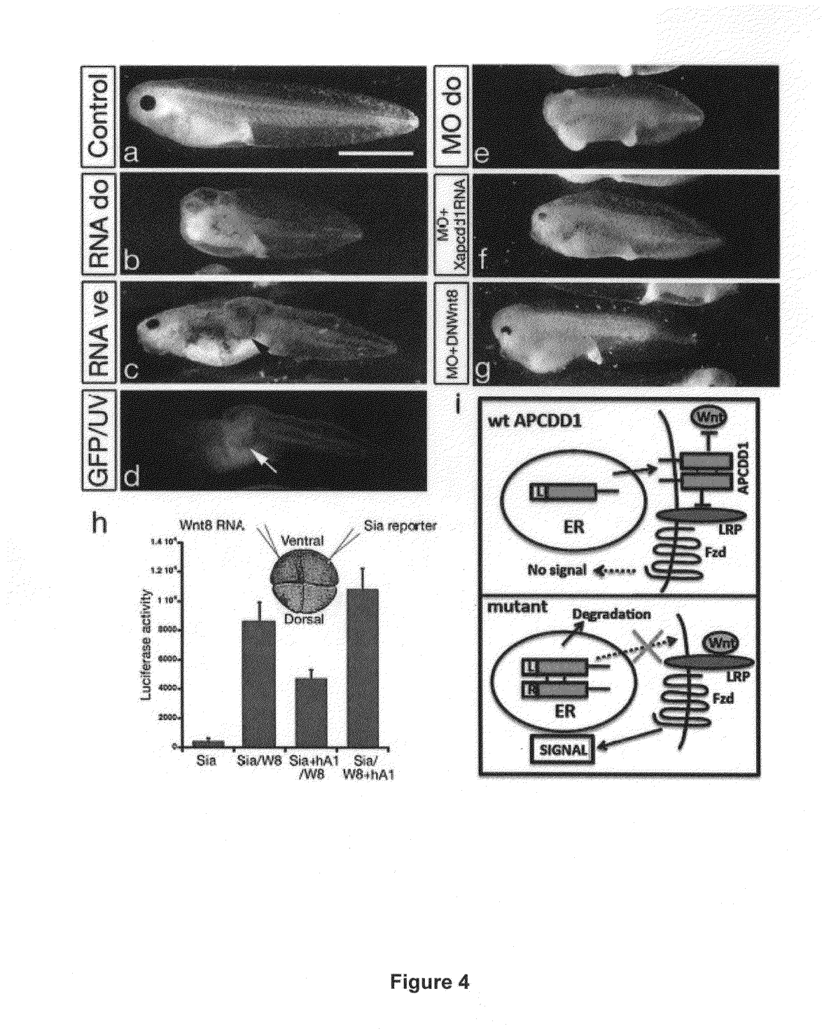

The maternal Wnt pathway, active on the dorsal side, is required for the formation of dorsal and anterior structures in early Xenopus laevis embryos. Overexpression of APCDD1 in dorsal blastomeres (n=35) reduced the anterior structures, such as the eyes and cement gland at the tadpole stage (FIG. 4a, b), consistent with maternal Wnt inhibition. APCDD1 also inhibited transcription of the siamois (Sia) reporter gene, which is activated by the maternal Wnt pathway (FIG. 2c). After the onset of zygotic transcription, a second endogenous Wnt pathway is activated on the ventral side of the embryo, and its inhibition produces secondary axes with incomplete heads. Ventral overexpression of APCDD1 induced secondary axes (n=43, 28% duplicated axes, FIG. 4c-e), consistent with an inhibitory effect on Wnt signaling. Direct inhibition of exogenous Wnt activity was also seen in transcription assays with Wnt RNA (Takahashi, M., et al. (2002) Cancer Res. 62, 5651-5656), but not with β-catenin (FIG. 2c), indicating that APCDD1 acts upstream of β-catenin in the Wnt pathway.

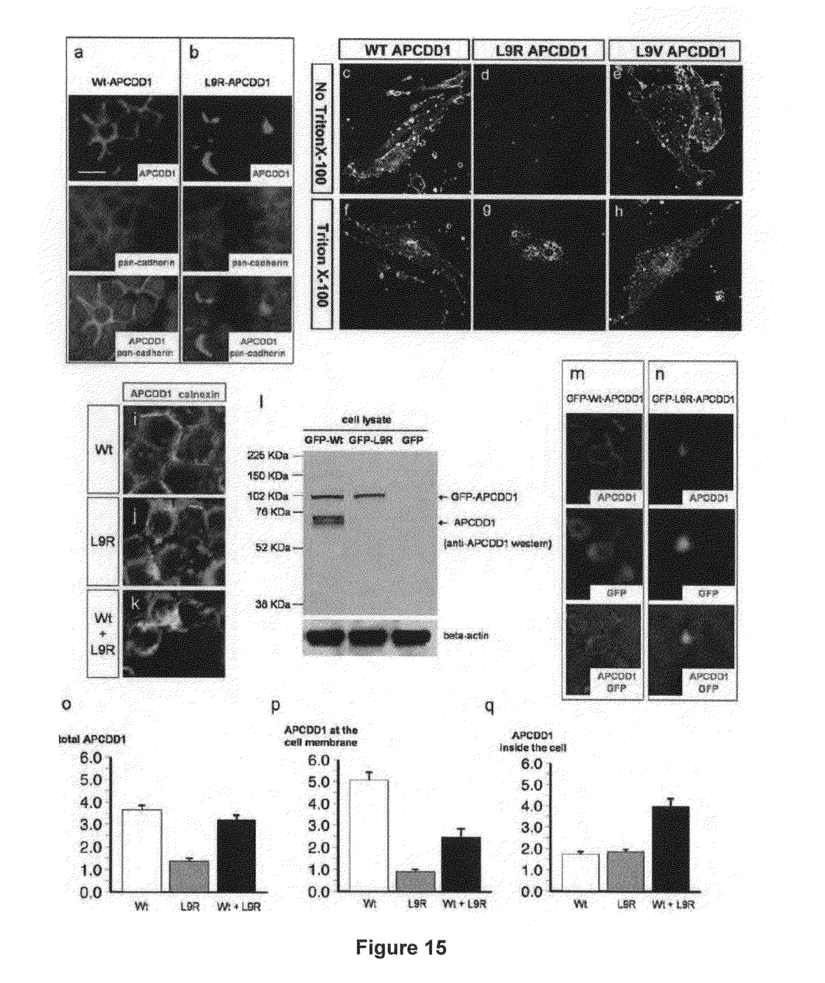

We next investigated which domain of APCDD1 mediates the Wnt inhibitory activity and the cells where APCDD1 functions to inhibit the pathway. Misexpression of mAPCDD1ΔTM (lacking the transmembrane domain) in the chick neural tube mimicked the effects observed with mAPCDD1 (FIGS. 12l-v and 13f-j). Secondly, APCDD1 could affect either the signaling cell, by regulating Wnt secretion, or the receiving cell. In Xenopus transcription assays where Wnt8 RNA injected in one cell activated the Sia reporter in an adjacent cell, APCDD1 RNA had an inhibitory effect when co-injected with the reporter, but not with Wnt8 (FIG. 4h), suggesting that APCDD1 inhibits the Wnt signaling cell autonomously in the receiving cell. Finally, since WT-APCDD1 contains a transmembrane domain (FIG. 1k), and was localized to the plasma membrane (FIG. 2h and FIG. 15a,c,f,i), we tested whether APCDD1 undergoes cleavage to generate a diffusible inhibitor (APCDD1ΔTM); however, it was undetectable in the medium of transfected cells (FIG. 9d). Collectively these data reveal that APCDD1 is likely a membrane-tethered Wnt inhibitor that acts as a dimer at the surface of the Wnt-receiving cell.

The L9R mutation disrupts the hydrophobic core of the signal peptide critical for co-translational processing of the APCDD1 protein (FIG. 8b, c) (Pidasheva, S., (2005) Hum. Mol. Genet. 14, 1679-1690). We analyzed protein stability and localization by western blotting and immunofluorescence, respectively, in two cell lines (HEK293T or Bend3.0) transfected with either wild type (WT) APCDD1 or two different mutant forms (pathogenic mutation L9R and conservative substitution L9V). Two fragments of 68 KDa and 130 KDa were detected in lysates of the WT- and the conservative mutant L9V-APCDD1transfected cells, whereas only a faint 68 KDa fragment was detected in that of the L9R mutant (FIG. 2f). WT- or L9V-APCDD1 protein was localized to the cell membrane, while the L9R-APCDD1 was retained within the endoplasmic reticulum (ER) (FIG. 2h, i and FIG. 15a-j). Furthermore, overexpression of an N-terminal GFP-tagged WT- or L9R-APCDD1 protein revealed that the mutant protein was not able to undergo cleavage or localize to the membrane (FIG. 15l-n). Finally, when the WT- and L9RAPCDD1 expression constructs were co-transfected either in cells or injected in Xenopus embryos, the wild type protein was degraded (FIG. 2e, g), and the remaining protein was mislocalized to the ER with the L9R isoform (FIG. 2j, k and FIG. 15k, o-q). Therefore, the L9R mutation likely functions in dominant-negative manner, to destabilize the WT protein and prevent it from reaching the plasma membrane.

We next tested if the L9R mutation affects the function of the protein in vivo, in chick and Xenopus. In the chick neural tube, expression of L9R-APCDD1 inhibited only weakly the transcription of eGFP transcription from the Wnt reporter (FIG. 10e-f) and had no effect on Sox3+ neural progenitors and neuronal subtypes (FIG. 3f-j and FIG. 11e-h), in contrast to the WT- or the L9V-APCDD1 (FIG. 11m-u). Moreover, L9R-APCDD1 was able to block the effect of the WT protein in the neural tube when they were co-transfected (FIG. 3k-o and FIG. 11i-l), indicative of a dominant-negative effect. The same results were observed in Xenopus, where the inhibitory effect of WT APCDD1 on Wnt8-induced transcription was blocked by coexpression of the L9R mutant (FIG. 2d).

We determined the consequences of XAPCDD1 (Xapcdd1) protein depletion on axis formation in Xenopus embryos. Xapcdd1 mRNA is expressed maternally throughout development with the highest level in animal (future ectoderm) and marginal (future mesoderm) cells of stage 10 embryos (FIG. 16a). Depletion of Xapcdd1 protein with a specific translation-blocking MO oligonucleotide (FIG. 16b) in the dorsal blastomeres of 4 cell-stage embryos resulted in the loss of anterior and dorsal structures (FIG. 4e and Table 2 below). This phenotype was rescued by either injection of MO-resistant 5′ mutant XAPCDD1 RNA, or by DNWnt8 RNA (FIG. 4e and Table 2), which inhibit endogenous zygotic Wnt signaling and its ventralizing effect. Therefore, the loss-of-function phenotype is consistent with ectopic activation on the dorsal side of a zygotic Wnt activity, and supports the notion that APCDD1 is a Wnt inhibitor.

TABLE 2
Phenotype of Xenopus apcdd1 depletion. Embryos were injected in both
dorsal blastomeres at 4 cells tage with 30 ng Xapcdd1 MO and scored
at stage 40 for antero-posterior development.

| | Normal | DAI 3-4 | | | |
|---|---|---|---|---|---|
| | (%) | (%) | DAI 1-2 (%) | Total | P |
| Control | 96 | 4 | — | 94 | |
| Xapcdd1 MO | — | 44 | 56 | 64 | <0.0001 |
| +Xapcdd1 RNA | — | 87 | 3 | 46 | <0.005* |
| +DNWnt8 RNA | — | 100 | — | 60 | <0.0001* |

DAI index: 5 is normal, 0 lacks all recognizable anterior and dorsal structures.
*= rescued embryos were compared to Xapcdd1-depleted embryos.

In conclusion, we suggest that APCDD1 prevents formation of the Wnt receptor complex (FIG. 4i) since it interacts in vitro with LRP5 and WNT3A. The L9R mutant is unable to repress Wnt-responsive genes, by trapping the Wt protein in the ER where it may undergo degradation (FIG. 4i).

Our findings underscore the requirement for exquisitely controlled regulation of the Wnt signaling pathway in HF morphogenesis and cycling. It is known that forced activation of Wnt signaling exclusively in the epidermis leads to increased hair follicle density and tumors (Gat, U., et al. (1998) Cell 95, 605-614). We postulate that in HHS, Wnt signaling is indirectly increased through loss of the inhibitory function of APCDD in both the epidermal and dermal compartments of the HF, although the lack of HHS scalp samples precluded us from verifying this assumption. This notion is supported by mice with targeted ablation of another Wnt inhibitor, Klotho, which exhibit a reduction in HF density due to indirect upregulation of Wnt signaling and a depletion of HF bulge stem cells (Liu, H., et al. (2007) Science 317, 803-806). Since APCDD1 is expressed in both epidermal HF cells as well as the dermal papilla, we postulate that the simultaneous deregulation of Wnt signaling in both compartments may lead to a reduction in organ size of the HF resulting in miniaturization.

Our study provides the first genetic evidence that mutations in a Wnt inhibitor result in hair loss in humans. APCDD1 may be implicated in polygenic HF disorders as well, since its resides within the linkage intervals on chromosome 18 in families with androgenetic alopecia (max NPL score=2.56) (Hillmer, A. M., et al. (2008) Genome Am. J. Hum. Genet. 82, 737-743) and alopecia areata (max LOD=3.93) (Martinez-Mir, A., et al. (2007) Am. J. Hum. Genet. 80, 316-328). Furthermore, since APCDD1 is expressed in a broad range of cell types (Jukkola, T., et al. (2004) Gene Expr. Patterns 4, 755-762), our findings raise the possibility that APCDD1 is involved in other Wnt regulated processes, such as early morphogenesis, stem cell renewal, neural development, and cancer.

### Example 2

### Materials and Methods

Animals. Homozygous Tie2GFP mice (strain 003658) were obtained from Jackson labs and bred to maintain homozygosity. Wnt7a^{+/-} (strain 001253) and Wnt7b^{+/-} (strain 004693) mice were obtained from Jackson labs, and interbred to generate embryos with different combinations of mutant alleles of these Wnt 7 genes. TOP-GAL transgenic mice were provided by Roel Nusse. Conditional β-catenin mutant mice (strain 004152) and Tek-Cre mice (strain 004128) were obtained from the Jackson Laboratory. Wild type C57bl6 and FVB mice were obtained from Charles River.

Adenoviral Injections. Adenoviruses expressing FC and soluble frizzled 8-FC fusions were generated by methods known in the art (Kuhnert, F. et al. (2004) Proc Natl Acad Sci USA 101, 266-71). Pregnant C57bl6 mice, 9 days gestation, were administered 5×10⁸ pfu of adenovirus via tail vein injection, and embryos were isolated 3 days later.

Immunohistochemistry. Embryos were isolated and fixed for 1 hour in 4% paraformaldehyde. Fixed tissue was submerged in 30% sucrose and then embedded in 2:1 30% sucrose:OCT mixture. 10 micron tissue cryosections were then generated and stained as follows. Sections were incubated in a blocking/permeabilization solution containing 50% goat serum and 0.2% Triton X-100 for 30 minutes at room temperature, followed by incubation in primary antibody solution overnight at 4° C. Appropriate secondary antibodies, conjugated to alexa fluorophores, were then incubated for 2 hours at room temperature prior to mounting in vectashield with DAPI (Vector H 1200). When applicable, fluorescein conjugated-BSL (Vector, FL-1101 diluted 1:500) was added to the secondary antibody mix, and these samples were then post-fixed for 10 minutes with 4% paraformaldehyde to stabilize BSL. All sections were analyzed with a Nikon Eclipse E800 microscope and images were taken with a Diagnostics Instruments SPOT camera and analyzed by SPOT software. For TOP-GAL transgenic mice, tissue sections of e12.5, p0, p7 and adult mice were double-labeled with a rabbit anti-lacZ antibody (ICN) diluted 1:5000 and BSL-FITC (Vector, FL-1101) diluted 1:500. For conditional β-catenin mutants and littermate controls, tissue sections were stained with a rat anti-CD31 antibody (BD Pharmingen, 553370), rabbit anti-NG2 antibody (Chemicon, AB5320), rabbit anti-glut-1 antibody (Chemicon, AB1340) a rabbit antiserum against Pard6 that stained blood and BSL-FITC all diluted 1:500. Adenoviral injected mice were stained with BSL-FITC, and with the rat anti-CD31 antibody.

In situ Hybridizations. The in situ hybridizations were performed following methods known in the art (Schaeren-Wiemers, N. & Gerfin-Moser, A. (1993) Histochemistry 100, 431-40) with few modifications that include incubation of fixed embryonic tissues with proteinase K for 5 minutes. cDNAs were either amplified from purified endothelial cells or they were obtained from Open Biosystems.

Migration Assays. A mouse brain endothelial cell line (bEND3.0 cells from ATCC) was grown in DMEM, with pen/strep, sodium pyruvate, glutamate, insulin and 10% FCS. Upon reaching confluency, the cells were starved of serum for 5 hours and then trypsinized and plated at 10⁵ cells/insert in a BD BioCoat Angiogensis System: Endothelial Cell Migration Plate (BD Biosciences 354144), with basal media (bEND3.0 media without FCS) alone, or containing 10 ng/ml VEGF (BD 354107), 0.5 ug/ml Wnt (R&D 3008-WN), or both VEGF and Wnt, in the wells beneath the insert. Cells were incubated for 20 hours, and then stained with 4 ug/ml Calcein AM (BD 354216). Endothelial cell migration was calculated by determining the fluorescence in the bottom well using a fluorescence plate reader with bottom reading capabilities.

### Results

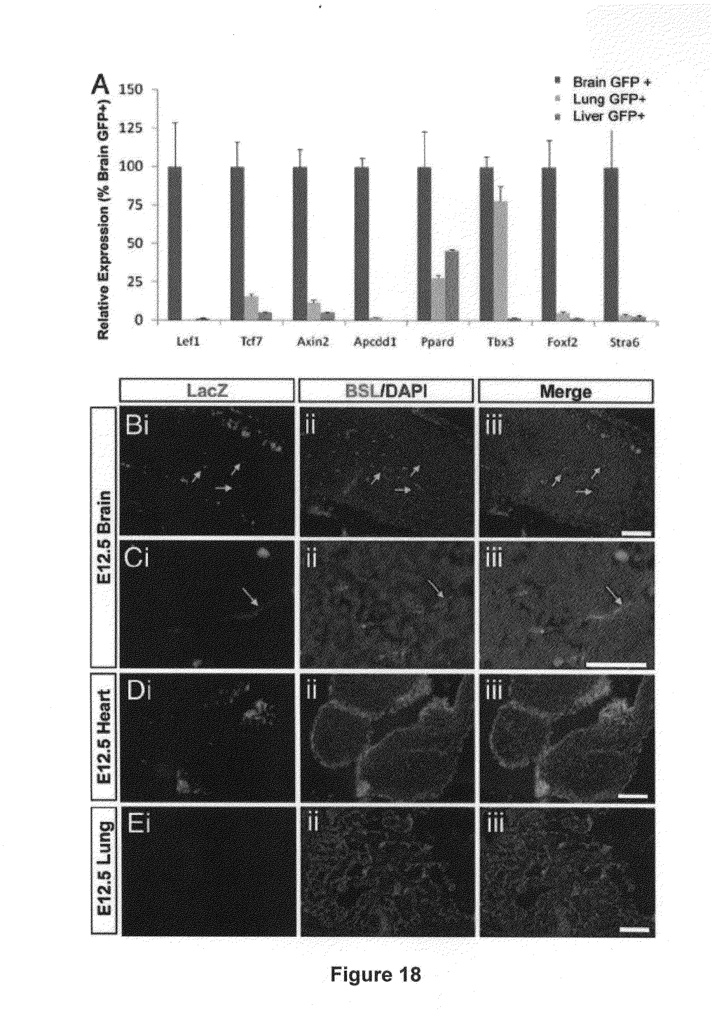

Wnt Signaling is Activated in CNS Vessels During Embryogenesis. To confirm that Wnt signaling is indeed activated specifically in CNS vessels, we analyzed tissue sections from mice expressing the Wnt reporter TOP-GAL transgene. These transgenic mice express the lacZ gene under the control of Tcf promoters and thus synthesize lacZ only in cells in which canonical Wnt/β-catenin signaling is activated (Hens J R, et al. (2005) J Bone Miner Res. 20:1103-1113). During the development of the murine CNS, angiogenesis is initiated at E10, as endothelial cells from the perineural vascular plexus invade the underlying neural tissue. We therefore double stained tissue sections of E12.5 TOP-Gal mice with an anti-LacZ antibody and the vascular marker Bandeiraea simplicifolia lectin I (BSL) (FIG. 18). We observed activated Wnt signaling, as evidenced by anti-lacZ immunostaining, in many different tissues during embryogenesis. LacZ expression, however, co-localized with the vascular marker BSL only in the CNS, but not in peripheral tissues including the heart, liver, and lung (FIG. 18B-E). These data demonstrate that Wnt signaling is specifically activated in CNS blood vessels during development.

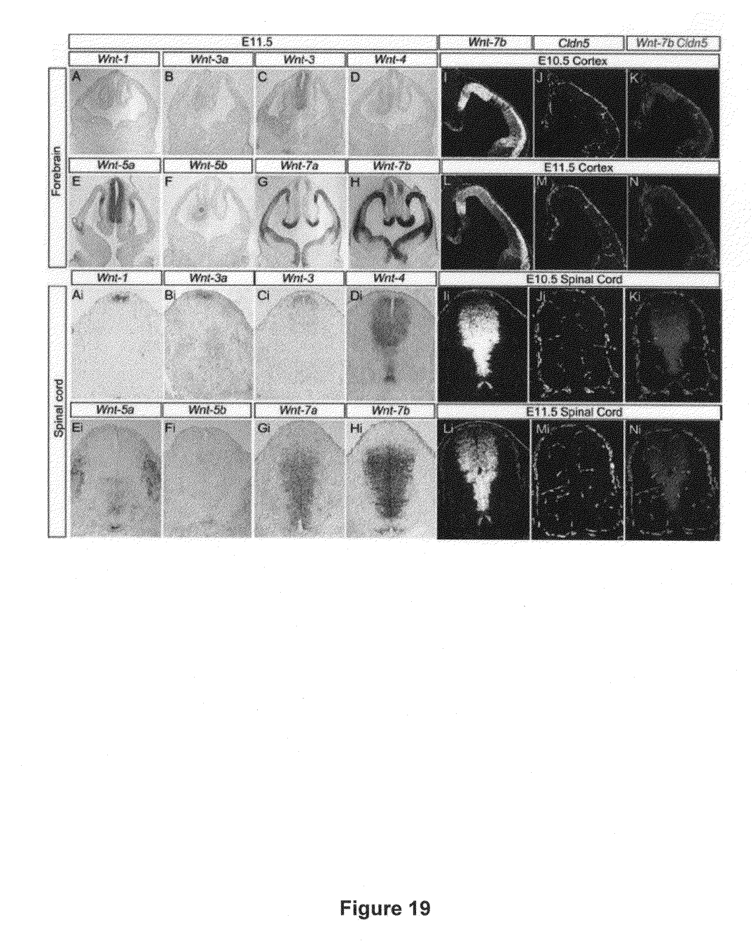

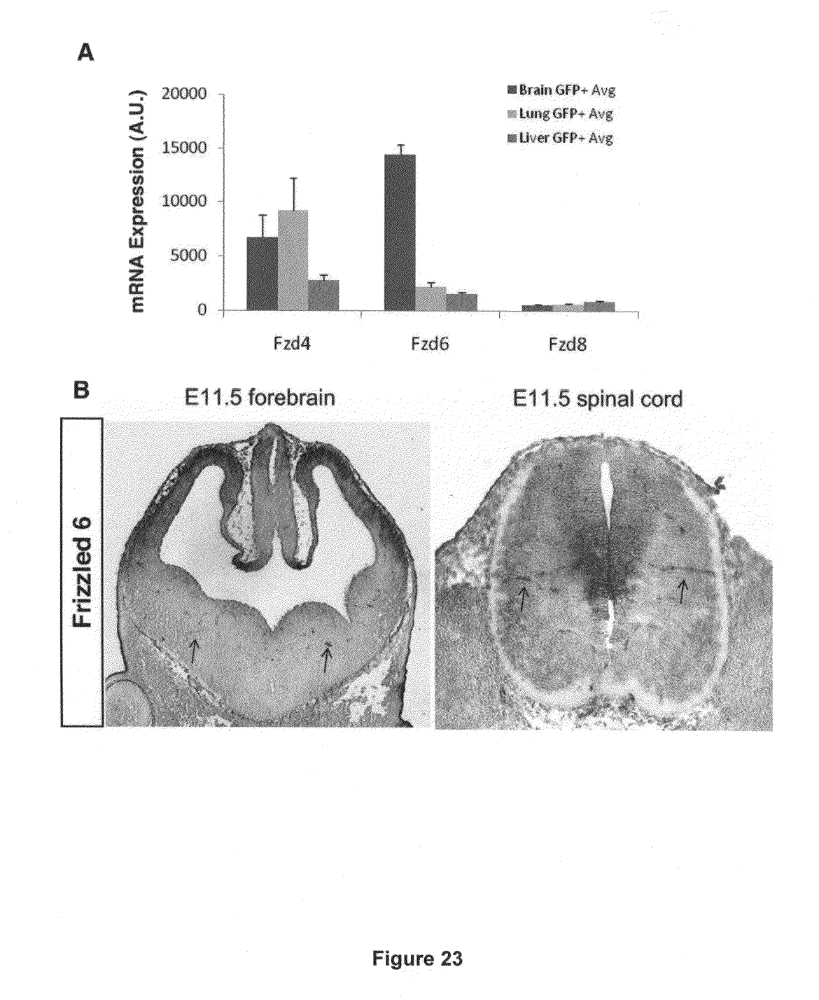

Active Wnt/β-catenin signaling in the CNS vasculature correlates with Wnt expression in neural progenitors and Frizzled expression in blood vessels. To identify which Wnt ligands may signal to CNS vessels, we next performed in situ hybridization studies using probes for various Wnt ligand mRNAs. We found that several canonical Wnt ligands were expressed by neural progenitors in the ventricular zone of the developing mouse CNS. In particular, vascular Wnt activation temporally correlated with the expression of Wnt7a and Wnt7b in the developing forebrain and in the ventral and intermediate spinal cord; Wnt 4 in the dorsal and intermediate spinal cord; and Wnt1, Wnt3, and Wnt3a throughout the dorsal neural tube (FIG. 18). As our analysis of TOP-GAL Wnt reporter mice demonstrated Wnt/β-catenin activity in endothelial cells throughout the CNS, this reporter activity is likely activated by different Wnt ligands expressed in spatially distinct regions of the CNS. In addition, neural progenitor cells in some CNS regions also expressed Wnt ligands that act through non-canonical signaling, including Wnt5a and Wnt5b (FIG. 19) These ligands may also be able to activate canonical β-catenin signaling depending on the Frizzled receptor type(s) expressed by the endothelial cells (Mikels A J, Nusse R (2006) PLoS Biol. 4:e115). Conversely, CNS endothelial cells express the Wnt receptors Frizzled 4, Frizzled 6, and Frizzled 8, as identified by microarray analysis of purified endothelial cells (FIG. 23). Frizzled 6 expression is highly enriched in CNS endothelial cells compared to the endothelial cells of the liver and lung (FIG. 23). Analysis of Wnt7b/Claudin 5 double fluorescent in situ hybridizations demonstrate that the capillary bed is largely formed in regions of the developing forebrain and spinal cord with high Wnt7b expression (FIG. 19l-N, Ii-Ni). Taken together, these data suggest that canonical Wnt/β-catenin signaling mediates endothelial-neural progenitor cellular interactions in the developing CNS.

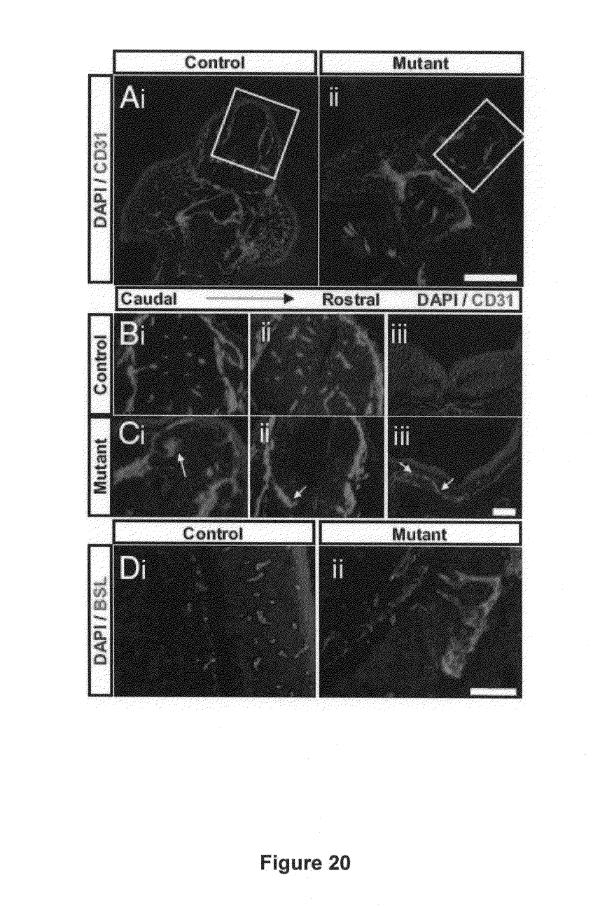

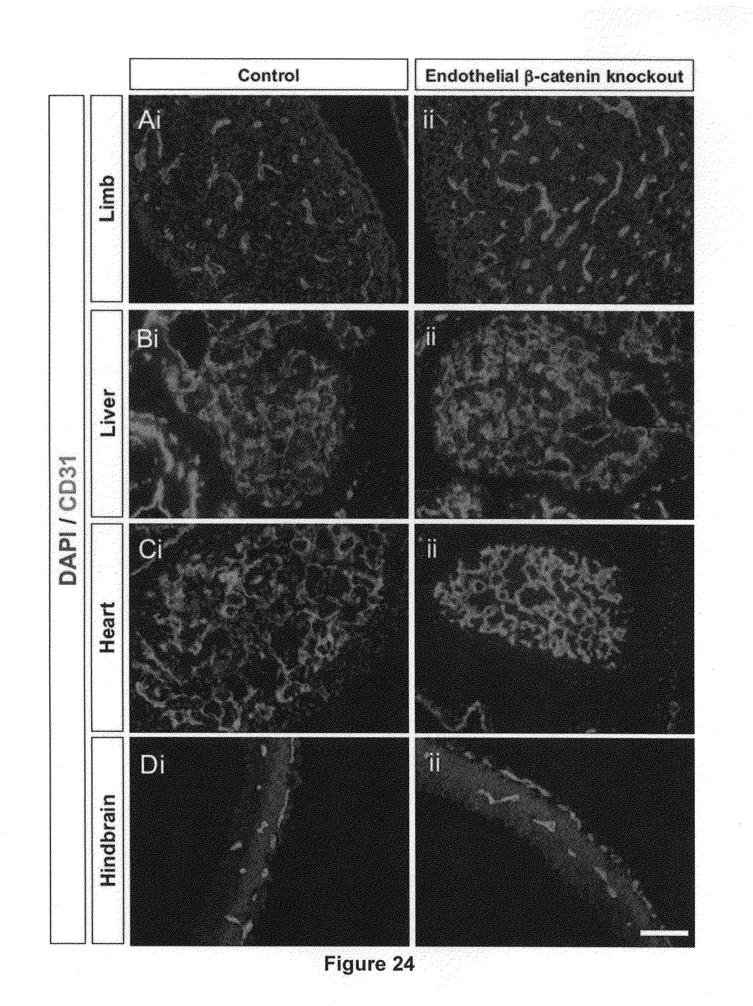

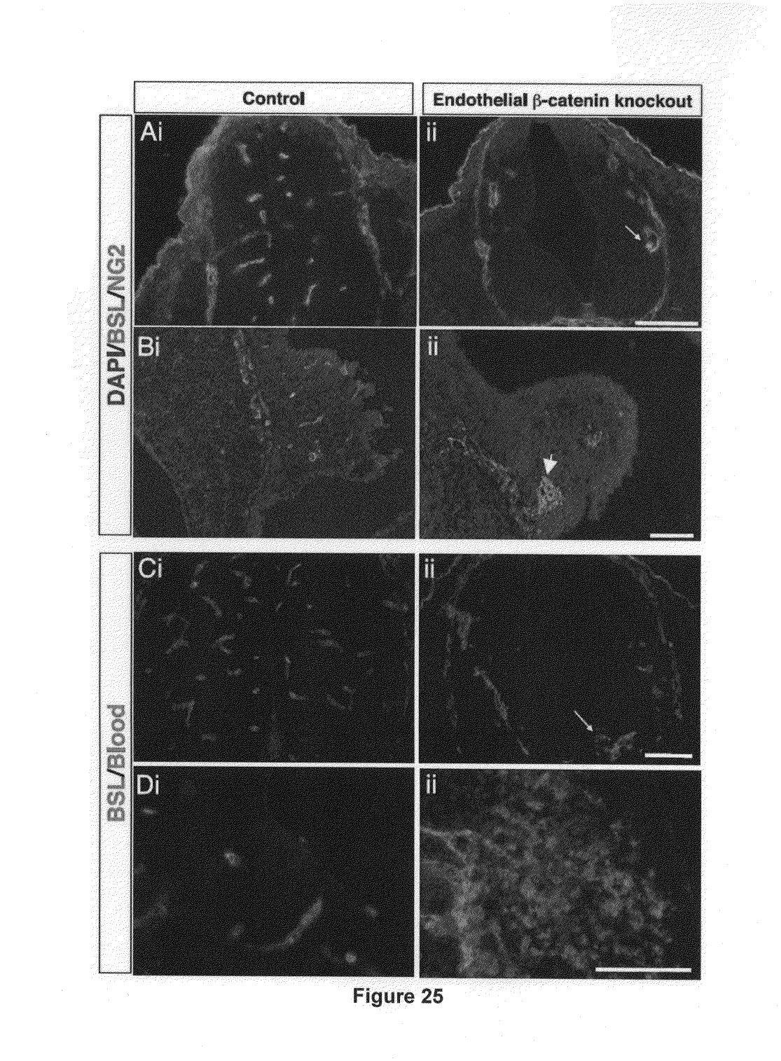

β-catenin is Required for CNS Vessel Formation In vivo. To determine whether β-catenin is required for CNS vessel formation, endothelial-specific β-catenin knockout mice were generated by using β-catenin^{flox/flox} and Tek1::cre mice. Tek1::cre mice express cre recombinase in endothelial cells throughout the body and therefore we used this method to delete β-catenin, an essential component of canonical Wnt signaling, from all vessels. β-catenin^{flox/flox}; Tek1::cre mice have been previously generated and die by E12.5 displaying mild patterning defects in the large vessels of the vitalline, umbilical cord, and head. The tissue capillary beds of these mutants appear normal, however, their CNS vasculature has not yet been examined (Cattelino A, et al. (2003) J Cell Biol. 162:1111-1122). We therefore next examined the vascular pattern of E11.5 endothelial-specific β-catenin mutants (β-catenin^{flox/flox}; Tek1::cre genotype) and litter mate controls (β-catenin^{flox/+}; Tek-cre and β-catenin^{flox/flox} genotypes) by immunostaining tissue sections with an anti-CD31 antibody to label all endothelial cells. We observed an overtly normal vascular pattern in non-neural tissues, including the liver, lung, skin, jaw, and tail, in all genotypes examined, but found major vascular defects in the CNS of all mutant mice examined (FIG. 20, FIG. 24, FIG. 25). Virtually no capillaries formed throughout the developing forebrain and spinal cord of the mutant mice. Furthermore, the perineural vascular plexus was significantly thickened, suggesting that endothelial cells stalled in the meninges, unable to invade the CNS parenchyma (FIG. 20). In each mutant examined, we observed large malformed vessels that did invade the CNS parenchyma. These vessels, however, did not form discrete tubes or capillary networks, but instead remained as large aggregates of endothelial cells that were often associated with hemorrhage. The number and extent of these vascular malformations varied between animals and in different regions of the developing neural tube. In addition, the thickness of the neuroectodermal cell layer was significantly decreased in the mutants, leading to an increased ventricular volume. This defect is likely secondary to the vascular defects, as the conditional mutants have β-catenin deleted specifically within endothelial cells. The malformations consisted of multiple layers of CD31⁺ endothelial cells, instead of single cell tubes as seen in the capillary beds of control animals. In some cases, these aggregates formed layered tubes with lumens; whereas, in other cases, they appeared as multicellular balls with no discernible lumen (FIG. 25). In many cases the aggregates remained attached to the meninges, forming extended contacts with this vascular plexus. In most cases, the aggregates recruited pericytes, often surrounded by a layer of these mural cells (FIGS. 25A and B). The malformations were often associated with hemorrhage, which ranged from small leaks to massive bleeding into the parenchyma (FIGS. 25C and D). Unfortunately, due to the early embryonic lethality of these mice, a complete map of β-catenin independent vessels remains unknown.

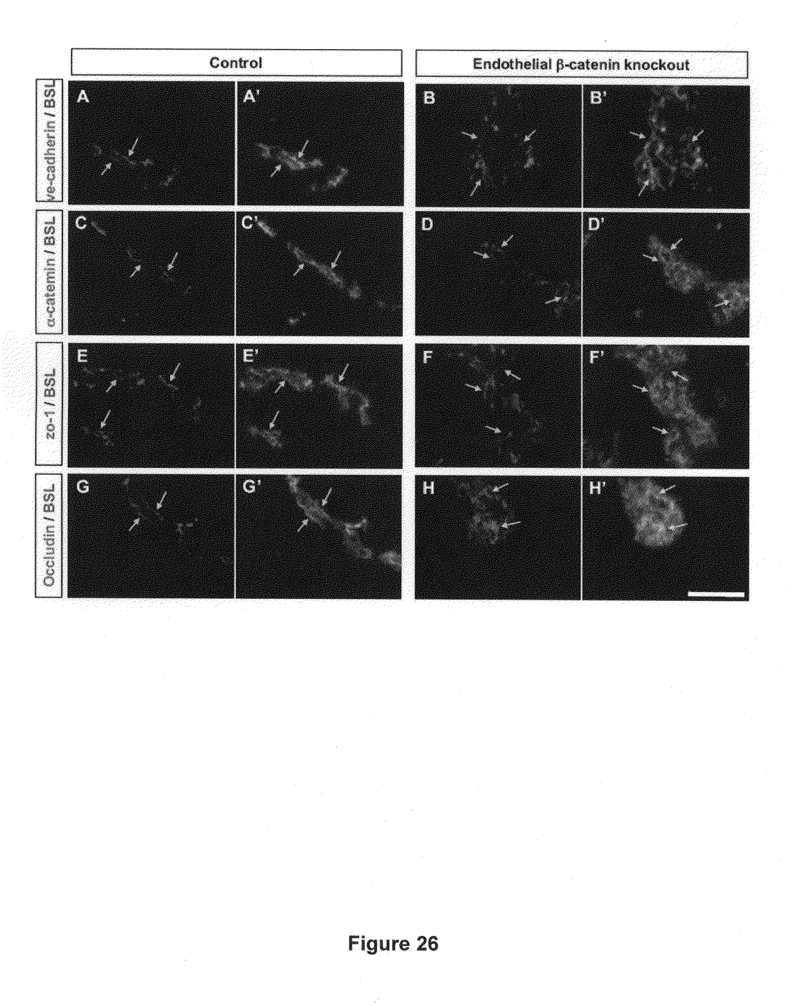

Taken together, the above findings demonstrate that β-catenin is required for the proper formation of CNS vessels, but not vessels in non-neural tissues. β-catenin functions not only as a transducer of Wnt signaling, but also as a component of the adherens junctions that join all endothelial cells. The vascular malformations observed in the endothelial-specific β-catenin mutants express other adherens junctions components at cellular junctions including α-catenin, γ-catenin and VE-cadherin as well as tight junction components ZO-1, Occludin, and Claudin 5 (See, for example, FIG. 26), suggesting that the defect is not due to incomplete junction protein expression. Although the expression of these proteins is unaffected, due to the cellular disorder of the malformations observed in these mice, the junctional components are also disordered. Adherens junctions connect endothelial cells in all tissues; however, the phenotype in the endothelial-specific β-catenin mutants is specific to the CNS matching the activation of Wnt signaling observed in the TOP-GAL mice.

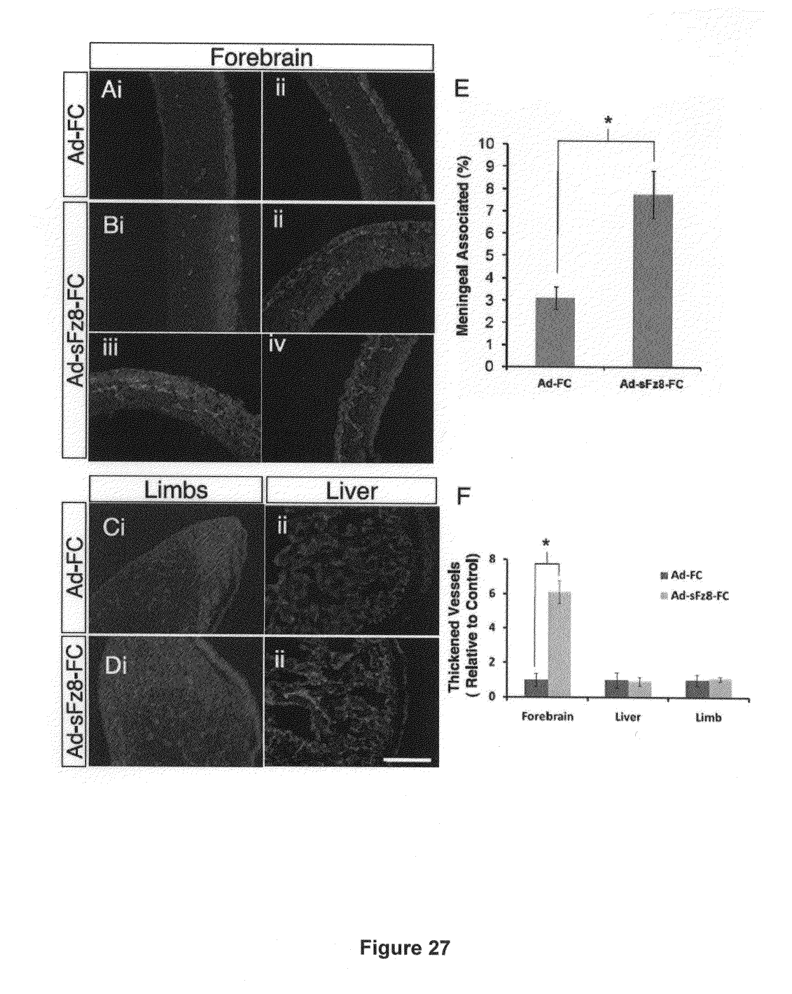

Blockade of Wnt inhibits CNS Angiogenesis. To further test whether the CNS-specific vascular defects in the endothelial specific β-catenin mutant were due to impaired Wnt signaling, and not other functions of β-catenin, we next examined the consequence of delivering a Wnt inhibitor to developing embryos. In this experiment, pregnant mice at 9 days of gestation were injected with adenoviruses encoding a soluble Frizzled 8-Fc fusion (Ad-sFz8-Fc) or a control Fc (Ad-Fc). After systemic injection, adenoviruses are taken up by liver cells, which then express the molecules encoded by the viruses (Kuhnert F, et al. (2004) Proc Natl Acad Sci USA. 101:266-271). A soluble Frizzled-8 ectodomain was used to bind extracellular Wnt ligands and Fc fusions were used to ensure delivery across the placenta. We observed many vascular malformations in the forebrains, but not non-neural tissue, of animals injected with Ad-sFz8-Fc but not when they were injected with the control Ad-Fc (FIG. 27). These malformations closely resembled those observed in the endothelial-specific β-catenin mutants, consisting of thickened tubes with multiple layers of endothelial cells and frequent meningeal attachment (FIGS. 27E and F). The phenotype produced by Ad-sFz8-Fc was less severe than in the endothelial-specific β-catenin mutants most likely because the amount of Ad-sFz8-Fc we could systemically deliver was limited by toxicity due to systemic (rather than specifically endothelial) Wnt signaling inhibition. This method inhibits Wnt-Frizzled interactions in all tissues, and thus vascular defects may be secondary to other developmental defects of inhibiting Wnt signaling. However, the similarity of the phenotype observed after Ad-sFz8-FC injection and endothelial-specific β-catenin depletion, suggests that the vascular defects are due to Wnt activity on CNS endothelial cells.

Wnt7a and Wnt7b are required for normal CNS angiogenesis. Which Wnts regulate CNS angiogenesis? Because Wnt7a and Wnt7b have the broadest expression pattern in ventral regions of the developing CNS, we next examined the vascular pattern of Wnt7a knockout, Wnt7b knockout, and Wnt7a; Wnt7b double knockout mice. Wnt7a knockout mice are viable and exhibit a normal vascular pattern at all ages tested including E10.5 and E12.5 (see, for example, FIG. 21). Wnt 7b knockout mice die by E11.5, and therefore due to the early lethality of this mutation we examined the spinal cord, which is vascularized before the forebrain during development. The ventral spinal cord of E10.5 Wnt7b knockout embryos displayed a decrease in capillary density, with vascular malformations that remained attached to the meningeal surface. In addition, the vascular plexus was thickened in many areas, similar to what was observed in the endothelial-specific β-catenin mutants (FIG. 21) The Wnt7a; Wnt7b double knockout mice also exhibited ventral vascular malformations and showed an even more severe thickening of the vascular plexus that often displayed extremely large dilations (FIG. 21). Due to the early lethality and thinning of the neural tissue in Wnt7b knockout mice, it is difficult to determine the extent of the capillary bed loss, however, the presence of vascular malformations and thickened vascular plexus is consistent with the defects observed in the endothelial-specific β-catenin mutants. In addition, as with the endothelial-specific β-catenin mutants, apparently normal vasculature was observed in the hindbrain of the Wnt7b knockout mice.

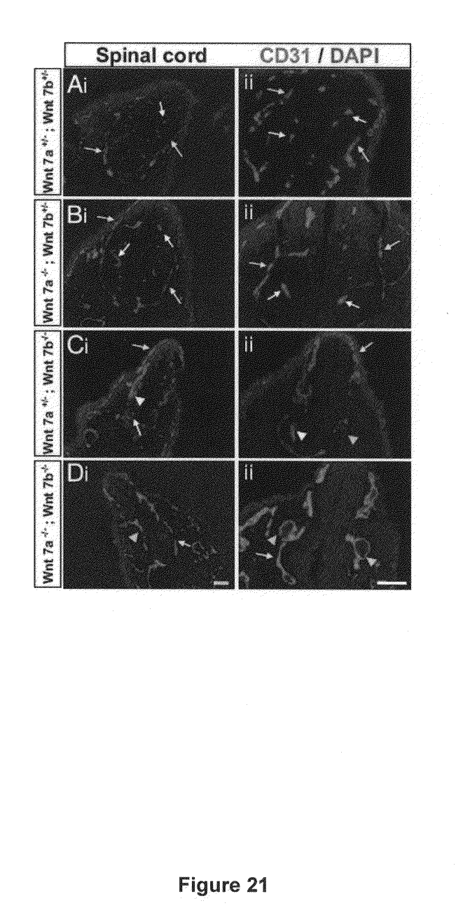

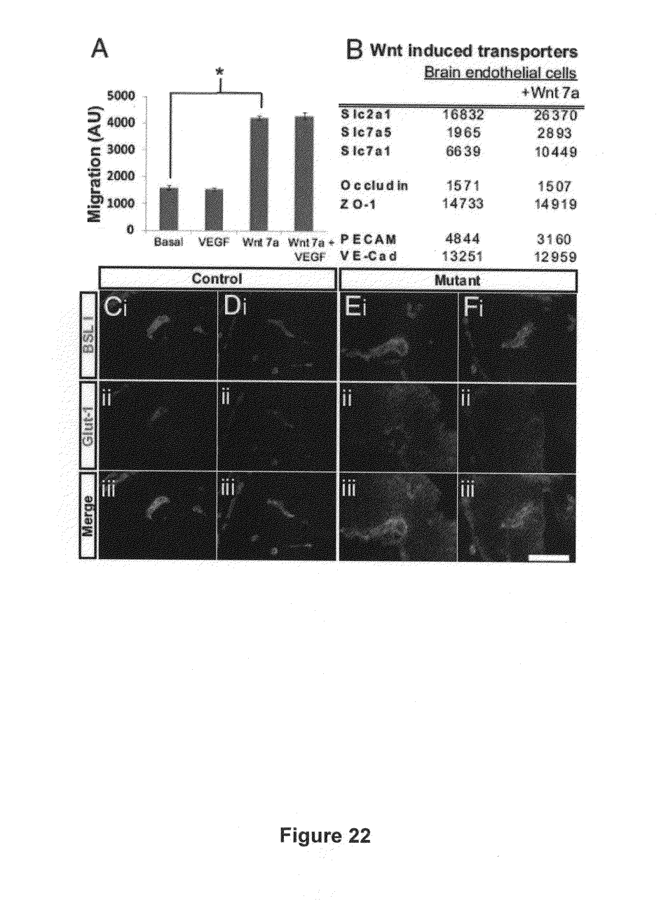

Because we observed similar malformed vessels following conditional depletion of β-catenin, delivery of Wnt inhibitors, and in Wnt mutants, we conclude that Wnt/β-catenin signaling is required for the formation of CNS vessels during embryogenesis. This is consistent with our genomic data in which several genes downstream of Wnt/β-catenin signaling are enriched in CNS endothelial cells compared with endothelial cells in non-neural tissues (FIG. 18A). The presence of vascular malformations that fail to invade the CNS after disruption of Wnt signaling suggests that Wnt may be a potent factor stimulating migration of endothelial cells into the CNS. To test whether this is the case, we measured the ability of Wnt7a to elicit CNS endothelial cell migration in vitro across a fibronectin-coated transwell system. Indeed, Wnt7a, but not VEGF, induced a strong migration of a mouse brain endothelial cell line (bEnd3.0 cells) across the filter (FIG. 22A). These results demonstrate that Wnts are a potent migration factor for CNS endothelial cells.

Wnt/β-catenin Signaling Regulates BBB specific properties of CNS endothelial cells. The fact that Wnt/β-catenin signaling regulates angiogenesis in the CNS, but not in other tissues, raises the possibility that this molecular signal imparts tissue-specific properties on the CNS endothelial cells to tightly couple CNS angiogenesis and BBB formation. CNS endothelial cells which form the BBB are characterized by the formation of tight junctions, and the expression of a variety of transporters both to provide selective transport of essential nutrients across the BBB into the brain and to efflux potential toxins from the brain.

To test whether Wnt signaling might regulate specific components of the BBB, we next used Affymetrix microarrays to examine the transcriptional profile of purified primary CNS endothelial cells cultured in the presence or absence of recombinant Wnt7a. Wnt7a increased the expression of several BBB-specific influx transporters, including Slc2a1 (Glut-1), Slc7a1 (CAT1), and Slc7a5 (TA1), but not tight junction molecules including Occludin and ZO-1 or pan-endothelial molecules including PECAM and VE-cadherin (FIG. 22B). To test the relevance of this in vivo, we examined the expression of Glut-1 in the endothelial-specific β-catenin mutants and wild-type littermates. In the CNS of wild-type embryos, Glut-1 displays specific expression in the vascular endothelial cells, whereas this expression is lost in endothelial cells in the endothelial-specific β-catenin mutants (FIG. 22C-F). Interestingly, the endothelial-specific β-catenin mutants exhibited an up-regulation of Glut-1 in the CNS parenchyma, likely a secondary response to lack of glucose transport from the blood as the conditional mutation of β-catenin is specific to the vascular endothelial cells. Along with the in vitro studies, these results demonstrate that Wnt/β-catenin regulates endothelial cell expression of the BBB-specific transporter Glut-1.

Our data demonstrates that Wnt/β-catenin signaling is essential for CNS angiogenesis and the expression of BBB-specific transport molecules. This provides evidence that angiogenesis and BBB formation are at least in part linked.

### Discussion

Wnt/β-catenin Signaling is Required for CNS, but not non-CNS, angiogenesis, and at least some aspects of BBB formation. Here we have used 3 methods to demonstrate that Wnt/β-catenin signaling is required specifically for the formation of CNS vessels in vivo. First, we demonstrate that conditional depletion of β-catenin, an essential component of canonical Wnt signaling, in all endothelial cells leads to severe CNS-specific angiogenesis defects. Second, we demonstrate that delivery of a soluble Frizzled ectodomain, a molecule which binds and inhibits Wnt ligands, produces a similar phenotype. Finally, we demonstrate that mutations in Wnt7 genes result in CNS-specific vascular malformations. Because inhibiting canonical Wnt signaling at multiple different steps in the pathway produces the same phenotype, we conclude that these CNS-specific angiogenesis defects are due to inhibition of Wnt/β-catenin signaling and not other functions of these molecules. The fact that these angiogenesis defects are CNS-specific demonstrates that distinct molecular mechanisms regulate CNS angiogenesis.

With regard to the relevant ligand-receptor pairs, we note that Norrin signaling through Frizzled 4 has been implicated in regulating retinal angiogenesis (Xu Q, et al. (2004) Cell 116:883-895). Our data demonstrates that Wnt/β-catenin signaling is required for angiogenesis throughout the CNS and suggests that different Wnt/Frizzled ligand-receptor pairs may mediate this response in spatially distinct regions of the CNS. Specifically, Wnt7a and Wnt7b are expressed in the forebrain and ventral neural tube, whereas Wnt1, Wnt3, Wnt3a, and Wnt4 are expressed in dorsal regions of the neural tube. In fact, our analysis of Wnt7a and Wnt7b mutants have identified that these genes are required for normal angiogenesis in ventral regions of the CNS, with Wnt7b likely more important. Frizzled6 is a likely candidate receptor as it is highly enriched in CNS blood vessels.

Our findings implicate neural stem cells and progenitors as important sources of angiogenic signals in the developing CNS, as we found that the expression of Wnt ligands in the ventricular zone of the neural tube correlates with the activation of β-catenin in the CNS vasculature. The interaction between neural stem cells and vascular cells has been hypothesized by the close association of these two cell populations in what is termed a “vascular niche” for neural stem cells. Reciprocal signals between these two cell populations have been observed in vitro. Endothelial cells signal to neural stem cells regulating their self-renewal and differentiation into neurons (see, e.g., Shen Q, et al. (2004) Science 304:1338-1340), whereas neural stem cells have been implicated in regulating the resistance of endothelial tight junctions (see, e.g., Weidenfeller C et al. (2007) J Neurochem. 101:555-565). The dramatic failure of embryonic brain growth that occurred when we blocked Wnt/β-catenin signaling is likely explained not only by a lack of blood flow due to suppression of angiogenesis but quite possibly also the loss of endothelial signals required to support neural stem cell survival and proliferation.

It is at least in part the case that the same signal that promotes angiogenesis into the CNS also drives morphogenesis into barrier forming vessels, as we have identified that Wnt7a regulates expression of the BBB-specific transporter glut-1 in purified endothelial cells in vitro, and β-catenin is necessary for its expression in vivo. Standard hypotheses describe BBB formation as a two step model in which angiogenesis is followed by barriergenesis. In this model, CNS vessels are first generated as leaky vessels, and then later in development are induced by neural cells to form a tight seal. Our findings suggest that the formation of leaky CNS blood vessels may be incompatible with developmental viability and that Wnt/β-catenin signaling functions to tightly couple angiogenesis to barriergenesis. Consistent with this, Wnts have also been implicated in regulating angiogenesis in the placenta and gonads in vivo (Monkley S J, et al. (1996) Development. 122:3343-3353; Jeays-Ward K, et al. (2003) Development. 130:3663-3670), both tissues where blood tissue barriers are critical to their physiological function.

### Example 3

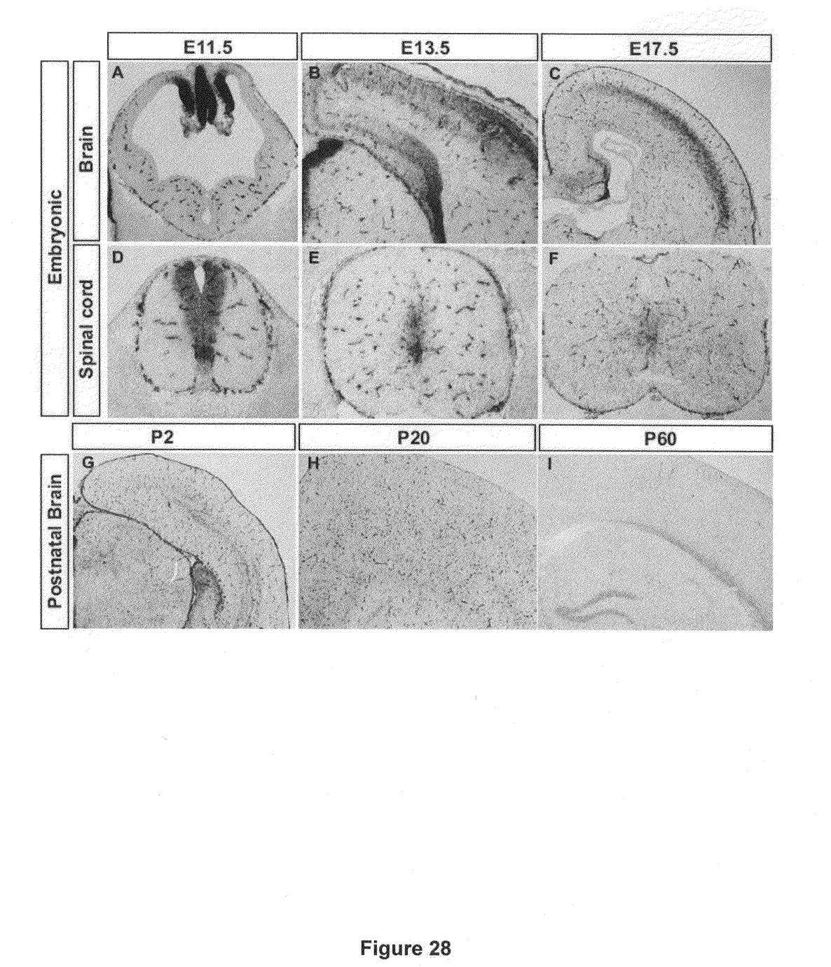

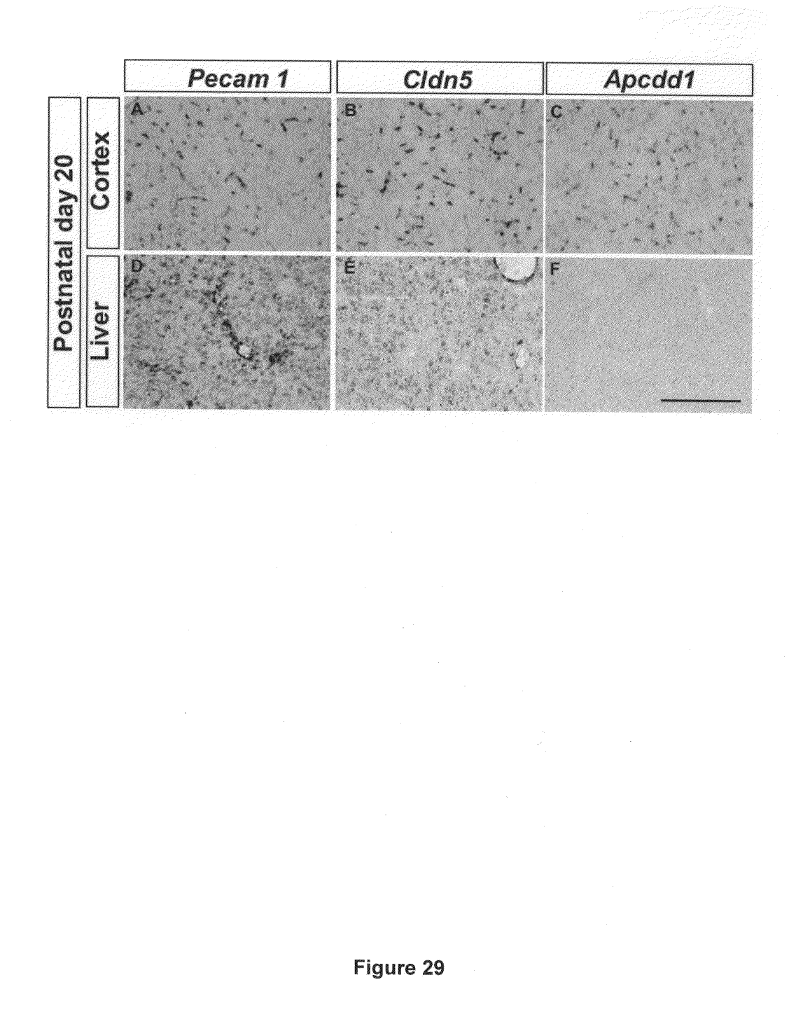

To determine if Apcdd1 may play a role in the Central Nervous System, Apcdd1 expression was evaluated in the developing and adult CNS. In situ hybridization of embryonic day 11.5 (E11.5), E13.5 and E17.5 mouse brain and spinal cord and postnatal day 2 (P2) and P20 mouse brain with an Apcdd1 antisense mRNA probe revealed that Apcdd1 is expressed in the vasculature of the developing brain and spinal cord (FIG. 28). Apcdd1 transcript is present in the vasculature of the developing CNS from the time when the vessels invade the CNS (E10.5-E11.0) through postnatal day 20 (P20) when the brain is fully mature in size, but is extinguished from the adult (P60) vasculature. In addition, Apcdd1 is expressed in non-vascular cells of the CNS in regions where there is active canonical Wnt signaling such as the cortical hem at E11.5 (A), the deep layer cortical neurons (B, C), the dentate gyrus (I) and the spinal cord progenitors (D-F). Interestingly, in situ hybridization with Apcdd1 antisense probes in the neocortex (FIG. 29A-C) and liver (FIG. 27D-F) at postnatal day 20 (P20) revealed that, in contrast to robust expression in the vasculature of the developing CNS, Apcdd1 is not expressed in the vasculature of other organs. This suggests that Apcdd1 expression in endothelial cells may be restricted to endothelial cells of the CNS vasculature.

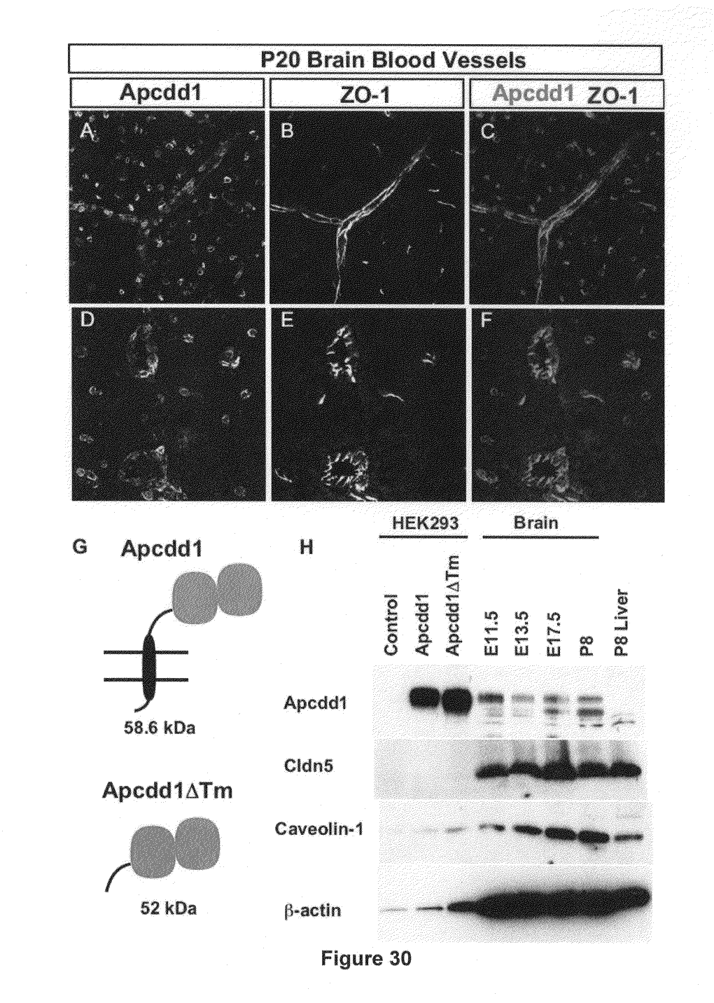

To determine if Apcdd1 protein is being made in the CNS, the presence of protein in developing brain tissue was assayed with an Apcdd-specific antibody by immunofluorescence and Western blot. Immunofluorescence of P20 brain tissue revealed that Apcdd1 protein is present in blood vessels and other CNS cell types (FIG. 30A, D). However, Apcdd1 protein does not co-localize with the tight junction protein ZO-1 (FIG. 30C, F), indicating that Apcdd1 does not function at the tight junctions between cells.

Western blots on protein extracts from E11.5, E13.5, E17.5, and P8 brain and P8 liver (FIG. 30H) revealed multiple bands were observed, with the shorter polypeptide appearing to be upregulated at later stages of development relative to the full length Apcdd1 polypeptide. This suggests that multiple Apcdd1 isoforms are expressed during brain development and that their expression is temporally regulated. To determine if any of these polypeptides could be secreted Apcdd1, Western blots were run alongside HEK293 cells that had been transfected with either a construct encoding full length Apcdd1 or a truncated Apcdd1 from which the transmembrane domain and cytoplasmic tail had been deleted (FIG. 30G). The shorter polypeptide observed in the brain tissue appeared to comigrate with the truncated Apcdd1 polypeptide, suggesting that the lower band observed was, in fact, secreted Apcdd1. Thus, it appears that both full-length and secreted Apcdd1 may play a role in the development of the CNS vasculature.

### Example 4

The human APCDD1 and APCDD1L proteins are very similar in amino acid sequence. To determine if APCDD1L functions in a similar manner to APCDD1 in inhibiting Wnt activity in vivo, we analyzed the neuronal specification in the developing chick spinal cord after transfection with the human APCDD1L. In the chick spinal cord, a Wnt/β-catenin gradient promotes proliferation of neural progenitors and generation of some neuronal classes. Transfection of the Wnt reporter TOP::eGFP in the chick neural tube revealed strong activation of the pathway in the dorsal and intermediate progenitors, as evidenced by elevated eGFP expression levels as described in Example 1. Overexpression of human APCDD1L reduced these eGFP expression levels. In addition, the misexpression of human APCDD1L decreased the number of Sox3+ neural progenitors, as well as various neuronal subtypes of dorsal and ventral origin. This Wnt inhibitory effect of human APCDD1L was similar to that observed with the Wild-type or L9V human APCDD1 and much weaker than the Wnt inhibitory effect of the mouse Apcdd1, a closer ortholog of the chick protein. These findings are consistent with the hypothesis that both human APCDD1 and APCDD1L function as Wnt inhibitors in a similar manner

### Example 5

We generated an affinity-purified rabbit polyclonal anti-mouse Apcdd1 antibody by immunizing rabbits with the synthetic peptide, CQRPSDGSSPDRPEKRATSY (SEQ ID NO:154, corresponding to the C-terminus of the extracellular domain of the mouse Apcdd1 protein, aa residues 441-459) conjugated to KLH (Pierce, Rockford, Ill.). This region is completely conserved among mouse and human APCDD1 proteins and human APCDD1L. The antibody was affinity-purified from the serum using the Sulfolink immobilization column (Pierce). This antibody strongly recognized human APCDD1 protein in western blots and immunofluorescence of the APCDD1-transfected Bend3.0 or HEK293 cells and it recognized human APCDD1L in immunofluorescence of the APCDD1L-transfected Bend3.0 or HEK293 cells

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

That which is claimed is:

1. A method for inhibiting Wnt signaling in a cell, comprising:
contacting a cell with an effective amount of an Adenomatosis Polyposis Coli Down-regulated (Apcdd) polypeptide,
wherein Wnt signaling is inhibited.

2. The method according to claim 1, wherein the cell is in vitro.

3. The method according to claim 2, wherein the cell is a cancer cell.

4. The method according to claim 2, wherein the cell is an endothelial cell.

5. The method according to claim 1, wherein the cell is in vivo.

6. The method according to claim 5, wherein the cell is in the central nervous system (CNS).

7. The method according to claim 6, wherein the Apcdd polypeptide is formulated for delivery to the CNS.

8. A method for inhibiting angiogenesis in a cell derived from the CNS, comprising:
contacting the CNS-derived cell with an effect amount of a Wnt inhibitor such that angiogenesis is inhibited.

9. The method according to claim 8, wherein the Wnt inhibitor is a peptide, a nucleic acid, or a small molecule.

10. The method according to claim 9, wherein the peptide is an Apcdd peptide.

11. The method according to claim 10, wherein the cell is in vitro.

12. The method according to claim 11, wherein the cell is an endothelial cell.

13. The method according to claim 8, wherein the cell is in vivo.

14. The method according to claim 13, wherein the cell is an endothelial cell.

15. The method according to claim 13, further comprising the step of measuring angiogenesis.

16. The method according to claim 13, wherein the cell is in a human suffering from a Wnt-mediated disorder.

17. The method according to claim 16, wherein the disorder is diabetic retinopathy or age related macular degeneration.

18. An Apcdd polypeptide formulated for delivery to the CNS.
